SEQUENCE LISTING (424 sequences)

SEQ ID NO: 1 - protein, 5 aa, Artificial Sequence
  Cyclic peptide with His-Ala-Val cell adhesion recognition motif
CHAVC

SEQ ID NO: 2 - protein, 6 aa, Artificial Sequence
  Cyclic peptide with His-Ala-Val cell adhesion recognition motif
CHAVCY

SEQ ID NO: 3 - protein, 6 aa, Artificial Sequence
  Cyclic peptide with His-Ala-Val cell adhesion recognition motif
CHAVDC

SEQ ID NO: 4 - protein, 7 aa, Artificial Sequence
  Cyclic peptide with His-Ala-Val cell adhesion recognition motif
CHAVDIC

SEQ ID NO: 5 - protein, 8 aa, Artificial Sequence
  Cyclic peptide with His-Ala-Val cell adhesion recognition motif
CHAVDINC

SEQ ID NO: 6 - protein, 9 aa, Artificial Sequence
  Cyclic peptide with His-Ala-Val cell adhesion recognition motif
CHAVDINGC

SEQ ID NO: 7 - protein, 6 aa, Artificial Sequence
  Cyclic peptide with His-Ala-Val cell adhesion recognition motif
CAHAVC

SEQ ID NO: 8 - protein, 7 aa, Artificial Sequence
  Cyclic peptide with His-Ala-Val cell adhesion recognition motif
CAHAVDC

SEQ ID NO: 9 - protein, 8 aa, Artificial Sequence
  Cyclic peptide with His-Ala-Val cell adhesion recognition motif
CAHAVDIC

SEQ ID NO: 10 - protein, 8 aa, Artificial Sequence
  Cyclic peptide with His-Ala-Val cell adhesion recognition motif
CRAHAVDC

SEQ ID NO: 11 - protein, 8 aa, Artificial Sequence
  Cyclic peptide with His-Ala-Val cell adhesion recognition motif
CLRAHAVC

SEQ ID NO: 12 - protein, 9 aa, Artificial Sequence
  Cyclic peptide with His-Ala-Val cell adhesion recognition motif
CLRAHAVDC

SEQ ID NO: 13 - protein, 6 aa, Artificial Sequence
  Cyclic peptide with His-Ala-Val cell adhesion recognition motif
CSHAVC

SEQ ID NO: 14 - protein, 7 aa, Artificial Sequence
  Cyclic peptide with His-Ala-Val cell adhesion recognition motif
CFSHAVC

SEQ ID NO: 15 - protein, 8 aa, Artificial Sequence
  Cyclic peptide with His-Ala-Val cell adhesion recognition motif
CLFSHAVC

SEQ ID NO: 16 - protein, 6 aa, Artificial Sequence
  Cyclic peptide with His-Ala-Val cell adhesion recognition motif
CHAVSC

SEQ ID NO: 17 - protein, 7 aa, Artificial Sequence
  Cyclic peptide with His-Ala-Val cell adhesion recognition motif
CSHAVSC

SEQ ID NO: 18 - protein, 8 aa, Artificial Sequence
  Cyclic peptide with His-Ala-Val cell adhesion recognition motif
CSHAVSSC

SEQ ID NO: 19 - protein, 7 aa, Artificial Sequence
  Cyclic peptide with His-Ala-Val cell adhesion recognition motif
CHAVSSC

SEQ ID NO: 20 - protein, 5 aa, Artificial Sequence
  Cyclic peptide with His-Ala-Val cell adhesion recognition motif
KHAVD

SEQ ID NO: 21 - protein, 5 aa, Artificial Sequence
  Cyclic peptide with His-Ala-Val cell adhesion recognition motif
DHAVK

SEQ ID NO: 22 - protein, 5 aa, Artificial Sequence
  Cyclic peptide with His-Ala-Val cell adhesion recognition motif
KHAVE

SEQ ID NO: 23 - protein, 6 aa, Artificial Sequence
  Cyclic peptide with His-Ala-Val cell adhesion recognition motif
AHAVDI

SEQ ID NO: 24 - protein, 7 aa, Artificial Sequence
  Cyclic peptide with His-Ala-Val cell adhesion recognition motif
SHAVDSS

SEQ ID NO: 25 - protein, 8 aa, Artificial Sequence
  Cyclic peptide with His-Ala-Val cell adhesion recognition motif
KSHAVSSD

SEQ ID NO: 26 - protein, 5 aa, Artificial Sequence
  Cyclic peptide with His-Ala-Val cell adhesion recognition motif
CHAVC

SEQ ID NO: 27 - protein, 5 aa, Artificial Sequence
  Cyclic peptide with His-Ala-Val cell adhesion recognition motif
CHAVC

SEQ ID NO: 28 - protein, 7 aa, Artificial Sequence
  Cyclic peptide with His-Ala-Val cell adhesion recognition motif
CHAVCSS

SEQ ID NO: 29 - protein, 7 aa, Artificial Sequence
  Cyclic peptide with His-Ala-Val cell adhesion recognition motif
SCHAVCS

SEQ ID NO: 30 - protein, 6 aa, Artificial Sequence
  Cyclic peptide with His-Ala-Val cell adhesion recognition motif
CHAVCT

SEQ ID NO: 31 - protein, 6 aa, Artificial Sequence
  Cyclic peptide with His-Ala-Val cell adhesion recognition motif
CHAVCE

SEQ ID NO: 32 - protein, 6 aa, Artificial Sequence
  Cyclic peptide with His-Ala-Val cell adhesion recognition motif
CHAVCD

SEQ ID NO: 33 - protein, 6 aa, Artificial Sequence
  Cyclic peptide with His-Ala-Val cell adhesion recognition motif
CHAVYC

SEQ ID NO: 34 - protein, 6 aa, Artificial Sequence
  Cyclic peptide with His-Ala-Val cell adhesion recognition motif
CHAVCY

SEQ ID NO: 35 - protein, 5 aa, Artificial Sequence
  Cyclic peptide with His-Ala-Val cell adhesion recognition motif
CHAVC

SEQ ID NO: 36 - protein, 5 aa, Artificial Sequence
  Cyclic peptide with His-Ala-Val cell adhesion recognition motif
CHAVC

SEQ ID NO: 37 - protein, 5 aa, Artificial Sequence
  Cyclic peptide with His-Ala-Val cell adhesion recognition motif
CHAVX

SEQ ID NO: 38 - protein, 5 aa, Artificial Sequence
  Cyclic peptide with His-Ala-Val cell adhesion recognition motif
XHAVC

SEQ ID NO: 39 - protein, 6 aa, Artificial Sequence
  Cyclic peptide with His-Ala-Val cell adhesion recognition motif
CHAVPC

SEQ ID NO: 40 - protein, 6 aa, Artificial Sequence
  Cadherin antagonist motif
XWVXXP

SEQ ID NO: 41 - protein, 4 aa, Artificial Sequence
  Cadherin antagonist containing Trp- cell adhesion recognition sequence
DWVI

SEQ ID NO: 42 - protein, 4 aa, Artificial Sequence
  Cadherin antagonist containing Trp- cell adhesion recognition sequence
DWVV

SEQ ID NO: 43 - protein, 4 aa, Artificial Sequence
  Cadherin antagonist containing Trp- cell adhesion recognition sequence
DWVM

SEQ ID NO: 44 - protein, 5 aa, Artificial Sequence
  Cadherin antagonist containing Trp- cell adhesion recognition sequence
DWVIP

SEQ ID NO: 45 - protein, 5 aa, Artificial Sequence
  Cadherin antagonist containing Trp- cell adhesion recognition sequence
DWVIA

SEQ ID NO: 46 - protein, 5 aa, Artificial Sequence
  Cadherin antagonist containing Trp- cell adhesion recognition sequence
DWVVP

SEQ ID NO: 47 - protein, 6 aa, Artificial Sequence
  Cadherin antagonist containing Trp- cell adhesion recognition sequence
DWVVPP

SEQ ID NO: 48 - protein, 6 aa, Artificial Sequence
  Cadherin antagonist containing Trp- cell adhesion recognition sequence
DWVVAP

SEQ ID NO: 49 - protein, 6 aa, Artificial Sequence
  Cadherin antagonist containing Trp- cell adhesion recognition sequence
DWVMPP

SEQ ID NO: 50 - protein, 6 aa, Artificial Sequence
  Cadherin antagonist containing Trp- cell adhesion recognition sequence
DWVMAP

SEQ ID NO: 51 - protein, 4 aa, Artificial Sequence
  Cadherin antagonist containing Trp- cell adhesion recognition sequence
EWVI

SEQ ID NO: 52 - protein, 4 aa, Artificial Sequence
  Cadherin antagonist containing Trp- cell adhesion recognition sequence
EWVV

SEQ ID NO: 53 - protein, 4 aa, Artificial Sequence
  Cadherin antagonist containing Trp- cell adhesion recognition sequence
EWVM

SEQ ID NO: 54 - protein, 5 aa, Artificial Sequence
  Cadherin antagonist containing Trp- cell adhesion recognition sequence
EWVIP

SEQ ID NO: 55 - protein, 5 aa, Artificial Sequence
  Cadherin antagonist containing Trp- cell adhesion recognition sequence
EWVIA

SEQ ID NO: 56 - protein, 5 aa, Artificial Sequence
  Cadherin antagonist containing Trp- cell adhesion recognition sequence
EWVVP

SEQ ID NO: 57 - protein, 6 aa, Artificial Sequence
  Cadherin antagonist containing Trp- cell adhesion recognition sequence
EWVVPP

SEQ ID NO: 58 - protein, 6 aa, Artificial Sequence
  Cadherin antagonist containing Trp- cell adhesion recognition sequence
EWVVAP

SEQ ID NO: 59 - protein, 6 aa, Artificial Sequence
  Cadherin antagonist containing Trp- cell adhesion recognition sequence
EWVMPP

SEQ ID NO: 60 - protein, 6 aa, Artificial Sequence
  Cadherin antagonist containing Trp- cell adhesion recognition sequence
EWVMAP

SEQ ID NO: 61 - protein, 7 aa, Artificial Sequence
  Cadherin antagonist containing Trp- cell adhesion recognition sequence
WVIWVIP

SEQ ID NO: 62 - protein, 4 aa, Artificial Sequence
  Cadherin antagonist containing Trp- cell adhesion recognition sequence
WVIA

SEQ ID NO: 63 - protein, 4 aa, Artificial Sequence
  Cadherin antagonist containing Trp- cell adhesion recognition sequence
WVVP

SEQ ID NO: 64 - protein, 4 aa, Artificial Sequence
  Cadherin antagonist containing Trp- cell adhesion recognition sequence
WVVA

SEQ ID NO: 65 - protein, 4 aa, Artificial Sequence
  Cadherin antagonist containing Trp- cell adhesion recognition sequence
WVMP

SEQ ID NO: 66 - protein, 4 aa, Artificial Sequence
  Cadherin antagonist containing Trp- cell adhesion recognition sequence
WVMA

SEQ ID NO: 67 - protein, 5 aa, Artificial Sequence
  Cadherin antagonist containing Trp- cell adhesion recognition sequence
WVIPP

SEQ ID NO: 68 - protein, 5 aa, Artificial Sequence
  Cadherin antagonist containing Trp- cell adhesion recognition sequence
WVIAP

SEQ ID NO: 69 - protein, 5 aa, Artificial Sequence
  Cadherin antagonist containing Trp- cell adhesion recognition sequence
WVVPP

SEQ ID NO: 70 - protein, 5 aa, Artificial Sequence
  Cadherin antagonist containing Trp- cell adhesion recognition sequence
WVVAP

SEQ ID NO: 71 - protein, 5 aa, Artificial Sequence
  Cadherin antagonist containing Trp- cell adhesion recognition sequence
WVMPP

SEQ ID NO: 72 - protein, 5 aa, Artificial Sequence
  Cadherin antagonist containing Trp- cell adhesion recognition sequence
WVMAP

SEQ ID NO: 73 - protein, 4 aa, Artificial Sequence
  Cadherin antagonist containing Trp- cell adhesion recognition sequence
DWII

SEQ ID NO: 74 - protein, 4 aa, Artificial Sequence
  Cadherin antagonist containing Trp- cell adhesion recognition sequence
DWIV

SEQ ID NO: 75 - protein, 4 aa, Artificial Sequence
  Cadherin antagonist containing Trp- cell adhesion recognition sequence
DWIM

SEQ ID NO: 76 - protein, 5 aa, Artificial Sequence
  Cadherin antagonist containing Trp- cell adhesion recognition sequence
DWIIP

SEQ ID NO: 77 - protein, 5 aa, Artificial Sequence
  Cadherin antagonist containing Trp- cell adhesion recognition sequence
DWIIA

SEQ ID NO: 78 - protein, 5 aa, Artificial Sequence
  Cadherin antagonist containing Trp- cell adhesion recognition sequence
DWIVP

SEQ ID NO: 79 - protein, 6 aa, Artificial Sequence
  Cadherin antagonist containing Trp- cell adhesion recognition sequence
DWIVPP

SEQ ID NO: 80 - protein, 6 aa, Artificial Sequence
  Cadherin antagonist containing Trp- cell adhesion recognition sequence
DWIVAP

SEQ ID NO: 81 - protein, 6 aa, Artificial Sequence
  Cadherin antagonist containing Trp- cell adhesion recognition sequence
DWIMPP

SEQ ID NO: 82 - protein, 6 aa, Artificial Sequence
  Cadherin antagonist containing Trp- cell adhesion recognition sequence
DWIMAP

SEQ ID NO: 83 - protein, 4 aa, Artificial Sequence
  Cadherin antagonist containing Trp- cell adhesion recognition sequence
EWII

SEQ ID NO: 84 - protein, 4 aa, Artificial Sequence
  Cadherin antagonist containing Trp- cell adhesion recognition sequence
EWIV

SEQ ID NO: 85 - protein, 4 aa, Artificial Sequence
  Cadherin antagonist containing Trp- cell adhesion recognition sequence
EWIM

SEQ ID NO: 86 - protein, 5 aa, Artificial Sequence
  Cadherin antagonist containing Trp- cell adhesion recognition sequence
EWIIP

SEQ ID NO: 87 - protein, 5 aa, Artificial Sequence
  Cadherin antagonist containing Trp- cell adhesion recognition sequence
EWIIA

SEQ ID NO: 88 - protein, 5 aa, Artificial Sequence
  Cadherin antagonist containing Trp- cell adhesion recognition sequence
EWIVP

SEQ ID NO: 89 - protein, 6 aa, Artificial Sequence
  Cadherin antagonist containing Trp- cell adhesion recognition sequence
EWIVPP

SEQ ID NO: 90 - protein, 6 aa, Artificial Sequence
  Cadherin antagonist containing Trp- cell adhesion recognition sequence
EWIVAP

SEQ ID NO: 91 - protein, 6 aa, Artificial Sequence
  Cadherin antagonist containing Trp- cell adhesion recognition sequence
EWIMPP

SEQ ID NO: 92 - protein, 6 aa, Artificial Sequence
  Cadherin antagonist containing Trp- cell adhesion recognition sequence
EWIMAP

SEQ ID NO: 93 - protein, 4 aa, Artificial Sequence
  Cadherin antagonist containing Trp- cell adhesion recognition sequence
WIIP

SEQ ID NO: 94 - protein, 4 aa, Artificial Sequence
  Cadherin antagonist containing Trp- cell adhesion recognition sequence
WIIA

SEQ ID NO: 95 - protein, 4 aa, Artificial Sequence
  Cadherin antagonist containing Trp- cell adhesion recognition sequence
WIVP

SEQ ID NO: 96 - protein, 4 aa, Artificial Sequence
  Cadherin antagonist containing Trp- cell adhesion recognition sequence
WIVA

SEQ ID NO: 97 - protein, 4 aa, Artificial Sequence
  Cadherin antagonist containing Trp- cell adhesion recognition sequence
WIMP

SEQ ID NO: 98 - protein, 4 aa, Artificial Sequence
  Cadherin antagonist containing Trp- cell adhesion recognition sequence
WIMA

SEQ ID NO: 99 - protein, 5 aa, Artificial Sequence
  Cadherin antagonist containing Trp- cell adhesion recognition sequence
WIIPP

SEQ ID NO: 100 - protein, 5 aa, Artificial Sequence
  Cadherin antagonist containing Trp- cell adhesion recognition sequence
WIIAP

SEQ ID NO: 101 - protein, 5 aa, Artificial Sequence
  Cadherin antagonist containing Trp- cell adhesion recognition sequence
WIVPP

SEQ ID NO: 102 - protein, 5 aa, Artificial Sequence
  Cadherin antagonist containing Trp- cell adhesion recognition sequence
WIVAP

SEQ ID NO: 103 - protein, 5 aa, Artificial Sequence
  Cadherin antagonist containing Trp- cell adhesion recognition sequence
WIMPP

SEQ ID NO: 104 - protein, 5 aa, Artificial Sequence
  Cadherin antagonist containing Trp- cell adhesion recognition sequence
WIMAP

SEQ ID NO: 105 - protein, 4 aa, Artificial Sequence
  Cadherin antagonist containing Trp- cell adhesion recognition sequence
DWLI

SEQ ID NO: 106 - protein, 4 aa, Artificial Sequence
  Cadherin antagonist containing Trp- cell adhesion recognition sequence
DWLV

SEQ ID NO: 107 - protein, 4 aa, Artificial Sequence
  Cadherin antagonist containing Trp- cell adhesion recognition sequence
DWLM

SEQ ID NO: 108 - protein, 5 aa, Artificial Sequence
  Cadherin antagonist containing Trp- cell adhesion recognition sequence
DWLIP

SEQ ID NO: 109 - protein, 5 aa, Artificial Sequence
  Cadherin antagonist containing Trp- cell adhesion recognition sequence
DWLIA

SEQ ID NO: 110 - protein, 5 aa, Artificial Sequence
  Cadherin antagonist containing Trp- cell adhesion recognition sequence
DWLVP

SEQ ID NO: 111 - protein, 6 aa, Artificial Sequence
  Cadherin antagonist containing Trp- cell adhesion recognition sequence
DWLVPP

SEQ ID NO: 112 - protein, 6 aa, Artificial Sequence
  Cadherin antagonist containing Trp- cell adhesion recognition sequence
DWLVAP

SEQ ID NO: 113 - protein, 6 aa, Artificial Sequence
  Cadherin antagonist containing Trp- cell adhesion recognition sequence
DWLMPP

SEQ ID NO: 114 - protein, 6 aa, Artificial Sequence
  Cadherin antagonist containing Trp- cell adhesion recognition sequence
DWLMAP

SEQ ID NO: 115 - protein, 4 aa, Artificial Sequence
  Cadherin antagonist containing Trp- cell adhesion recognition sequence
EWLI

SEQ ID NO: 116 - protein, 4 aa, Artificial Sequence
  Cadherin antagonist containing Trp- cell adhesion recognition sequence
EWLV

SEQ ID NO: 117 - protein, 4 aa, Artificial Sequence
  Cadherin antagonist containing Trp- cell adhesion recognition sequence
EWLM

SEQ ID NO: 118 - protein, 5 aa, Artificial Sequence
  Cadherin antagonist containing Trp- cell adhesion recognition sequence
EWLIP

SEQ ID NO: 119 - protein, 5 aa, Artificial Sequence
  Cadherin antagonist containing Trp- cell adhesion recognition sequence
EWLIA

SEQ ID NO: 120 - protein, 5 aa, Artificial Sequence
  Cadherin antagonist containing Trp- cell adhesion recognition sequence
EWLVP

SEQ ID NO: 121 - protein, 6 aa, Artificial Sequence
  Cadherin antagonist containing Trp- cell adhesion recognition sequence
EWLVPP

SEQ ID NO: 122 - protein, 6 aa, Artificial Sequence
  Cadherin antagonist containing Trp- cell adhesion recognition sequence
EWLVAP

SEQ ID NO: 123 - protein, 6 aa, Artificial Sequence
  Cadherin antagonist containing Trp- cell adhesion recognition sequence
EWLMPP

SEQ ID NO: 124 - protein, 6 aa, Artificial Sequence
  Cadherin antagonist containing Trp- cell adhesion recognition sequence
EWLMAP

SEQ ID NO: 125 - protein, 4 aa, Artificial Sequence
  Cadherin antagonist containing Trp- cell adhesion recognition sequence
WLIP

SEQ ID NO: 126 - protein, 4 aa, Artificial Sequence
  Cadherin antagonist containing Trp- cell adhesion recognition sequence
WLIA

SEQ ID NO: 127 - protein, 7 aa, Artificial Sequence
  Cadherin antagonist containing Trp- cell adhesion recognition sequence
WLVWLVP

SEQ ID NO: 128 - protein, 4 aa, Artificial Sequence
  Cadherin antagonist containing Trp- cell adhesion recognition sequence
WLVA

SEQ ID NO: 129 - protein, 4 aa, Artificial Sequence
  Cadherin antagonist containing Trp- cell adhesion recognition sequence
WLMP

SEQ ID NO: 130 - protein, 4 aa, Artificial Sequence
  Cadherin antagonist containing Trp- cell adhesion recognition sequence
WLMA

SEQ ID NO: 131 - protein, 5 aa, Artificial Sequence
  Cadherin antagonist containing Trp- cell adhesion recognition sequence
WLIPP

SEQ ID NO: 132 - protein, 5 aa, Artificial Sequence
  Cadherin antagonist containing Trp- cell adhesion recognition sequence
WLIAP

SEQ ID NO: 133 - protein, 5 aa, Artificial Sequence
  Cadherin antagonist containing Trp- cell adhesion recognition sequence
WLVPP

SEQ ID NO: 134 - protein, 5 aa, Artificial Sequence
  Cadherin antagonist containing Trp- cell adhesion recognition sequence
WLVAP

SEQ ID NO: 135 - protein, 5 aa, Artificial Sequence
  Cadherin antagonist containing Trp- cell adhesion recognition sequence
WLMPP

SEQ ID NO: 136 - protein, 5 aa, Artificial Sequence
  Cadherin antagonist containing Trp- cell adhesion recognition sequence
WLMAP

SEQ ID NO: 137 - protein, 4 aa, Artificial Sequence
  Cadherin antagonist containing Trp- cell adhesion recognition sequence
DWVL

SEQ ID NO: 138 - protein, 4 aa, Artificial Sequence
  Cadherin antagonist containing Trp- cell adhesion recognition sequence
DWIL

SEQ ID NO: 139 - protein, 4 aa, Artificial Sequence
  Cadherin antagonist containing Trp- cell adhesion recognition sequence
DWLL

SEQ ID NO: 140 - protein, 4 aa, Artificial Sequence
  Cadherin antagonist containing Trp- cell adhesion recognition sequence
EWVL

SEQ ID NO: 141 - protein, 4 aa, Artificial Sequence
  Cadherin antagonist containing Trp- cell adhesion recognition sequence
EWIL

SEQ ID NO: 142 - protein, 4 aa, Artificial Sequence
  Cadherin antagonist containing Trp- cell adhesion recognition sequence
EWLL

SEQ ID NO: 143 - protein, 5 aa, Artificial Sequence
  Cadherin antagonist containing Trp- cell adhesion recognition sequence
DWVLP

SEQ ID NO: 144 - protein, 5 aa, Artificial Sequence
  Cadherin antagonist containing Trp- cell adhesion recognition sequence
DWILP

SEQ ID NO: 145 - protein, 5 aa, Artificial Sequence
  Cadherin antagonist containing Trp- cell adhesion recognition sequence
DWLLP

SEQ ID NO: 146 - protein, 5 aa, Artificial Sequence
  Cadherin antagonist containing Trp- cell adhesion recognition sequence
EWVLP

SEQ ID NO: 147 - protein, 5 aa, Artificial Sequence
  Cadherin antagonist containing Trp- cell adhesion recognition sequence
EWILP

SEQ ID NO: 148 - protein, 5 aa, Artificial Sequence
  Cadherin antagonist containing Trp- cell adhesion recognition sequence
EWLLP

SEQ ID NO: 149 - protein, 5 aa, Artificial Sequence
  Cadherin antagonist containing Trp- cell adhesion recognition sequence
DWVLA

SEQ ID NO: 150 - protein, 5 aa, Artificial Sequence
  Cadherin antagonist containing Trp- cell adhesion recognition sequence
DWILA

SEQ ID NO: 151 - protein, 5 aa, Artificial Sequence
  Cadherin antagonist containing Trp- cell adhesion recognition sequence
DWLLA

SEQ ID NO: 152 - protein, 5 aa, Artificial Sequence
  Cadherin antagonist containing Trp- cell adhesion recognition sequence
EWVLA

SEQ ID NO: 153 - protein, 5 aa, Artificial Sequence
  Cadherin antagonist containing Trp- cell adhesion recognition sequence
EWILA

SEQ ID NO: 154 - protein, 5 aa, Artificial Sequence
  Cadherin antagonist containing Trp- cell adhesion recognition sequence
EWLLA

SEQ ID NO: 155 - protein, 6 aa, Artificial Sequence
  Cadherin antagonist containing Trp- cell adhesion recognition sequence
DWVLPP

SEQ ID NO: 156 - protein, 6 aa, Artificial Sequence
  Cadherin antagonist containing Trp- cell adhesion recognition sequence
DWILPP

SEQ ID NO: 157 - protein, 6 aa, Artificial Sequence
  Cadherin antagonist containing Trp- cell adhesion recognition sequence
DWLLPP

SEQ ID NO: 158 - protein, 6 aa, Artificial Sequence
  Cadherin antagonist containing Trp- cell adhesion recognition sequence
EWVLPP

SEQ ID NO: 159 - protein, 6 aa, Artificial Sequence
  Cadherin antagonist containing Trp- cell adhesion recognition sequence
EWILPP

SEQ ID NO: 160 - protein, 6 aa, Artificial Sequence
  Cadherin antagonist containing Trp- cell adhesion recognition sequence
EWLLPP

SEQ ID NO: 161 - protein, 6 aa, Artificial Sequence
  Cadherin antagonist containing Trp- cell adhesion recognition sequence
DWVLAP

SEQ ID NO: 162 - protein, 6 aa, Artificial Sequence
  Cadherin antagonist containing Trp- cell adhesion recognition sequence
DWILAP

SEQ ID NO: 163 - protein, 6 aa, Artificial Sequence
  Cadherin antagonist containing Trp- cell adhesion recognition sequence
DWLLAP

SEQ ID NO: 164 - protein, 6 aa, Artificial Sequence
  Cadherin antagonist containing Trp- cell adhesion recognition sequence
EWVLAP

SEQ ID NO: 165 - protein, 6 aa, Artificial Sequence
  Cadherin antagonist containing Trp- cell adhesion recognition sequence
EWILAP

SEQ ID NO: 166 - protein, 6 aa, Artificial Sequence
  Cadherin antagonist containing Trp- cell adhesion recognition sequence
EWLLAP

SEQ ID NO: 167 - protein, 4 aa, Artificial Sequence
  Cadherin antagonist containing Trp- cell adhesion recognition sequence
WVLP

SEQ ID NO: 168 - protein, 4 aa, Artificial Sequence
  Cadherin antagonist containing Trp- cell adhesion recognition sequence
WILP

SEQ ID NO: 169 - protein, 4 aa, Artificial Sequence
  Cadherin antagonist containing Trp- cell adhesion recognition sequence
WLLP

SEQ ID NO: 170 - protein, 4 aa, Artificial Sequence
  Cadherin antagonist containing Trp- cell adhesion recognition sequence
WVLA

SEQ ID NO: 171 - protein, 4 aa, Artificial Sequence
  Cadherin antagonist containing Trp- cell adhesion recognition sequence
WILA

SEQ ID NO: 172 - protein, 4 aa, Artificial Sequence
  Cadherin antagonist containing Trp- cell adhesion recognition sequence
WLLA

SEQ ID NO: 173 - protein, 5 aa, Artificial Sequence
  Cadherin antagonist containing Trp- cell adhesion recognition sequence
WVLPP

SEQ ID NO: 174 - protein, 5 aa, Artificial Sequence
  Cadherin antagonist containing Trp- cell adhesion recognition sequence
WILPP

SEQ ID NO: 175 - protein, 5 aa, Artificial Sequence
  Cadherin antagonist containing Trp- cell adhesion recognition sequence
WLLPP

SEQ ID NO: 176 - protein, 5 aa, Artificial Sequence
  Cadherin antagonist containing Trp- cell adhesion recognition sequence
WVLAP

SEQ ID NO: 177 - protein, 5 aa, Artificial Sequence
  Cadherin antagonist containing Trp- cell adhesion recognition sequence
WILAP

SEQ ID NO: 178 - protein, 5 aa, Artificial Sequence
  Cadherin antagonist containing Trp- cell adhesion recognition sequence
WLLAP

SEQ ID NO: 179 - protein, 6 aa, Artificial Sequence
  Cadherin antagonist containing Trp- cell adhesion recognition sequence
DWVIPP

SEQ ID NO: 180 - protein, 5 aa, Artificial Sequence
  Cadherin antagonist containing Trp- cell adhesion recognition sequence
DWVVA

SEQ ID NO: 181 - protein, 5 aa, Artificial Sequence
  Cadherin antagonist containing Trp- cell adhesion recognition sequence
EWVMP

SEQ ID NO: 182 - protein, 11 aa, Artificial Sequence
  Cadherin antagonist containing HAV binding motif cell adhesion recognition sequence
XFXIXXXXGXX

SEQ ID NO: 183 - protein, 11 aa, Artificial Sequence
  Cadherin antagonist containing HAV binding motif cell adhesion recognition sequence
WLXIXXXXGQI

SEQ ID NO: 184 - protein, 11 aa, Artificial Sequence
  Cadherin antagonist containing HAV binding motif
IFIINPISGQL

SEQ ID NO: 185 - protein, 11 aa, Artificial Sequence
  Cadherin antagonist containing HAV binding motif
IFILNPISGQL

SEQ ID NO: 186 - protein, 11 aa, Artificial Sequence
  Cadherin antagonist containing HAV binding motif
VFAVEKETGWL

SEQ ID NO: 187 - protein, 11 aa, Artificial Sequence
  Cadherin antagonist containing HAV binding motif
VFSINSMSGRM

SEQ ID NO: 188 - protein, 11 aa, Artificial Sequence
  Cadherin antagonist containing HAV binding motif
VFIIERETGWL

SEQ ID NO: 189 - protein, 11 aa, Artificial Sequence
  Cadherin antagonist containing HAV binding motif
VFTIEKESGWL

SEQ ID NO: 190 - protein, 11 aa, Artificial Sequence
  Cadherin antagonist containing HAV binding motif
VFNIDSMSGRM

SEQ ID NO: 191 - protein, 11 aa, Artificial Sequence
  Cadherin antagonist containing HAV binding motif
WLKIDSVNGQI

SEQ ID NO: 192 - protein, 11 aa, Artificial Sequence
  Cadherin antagonist containing HAV binding motif
WLKIDPVNGQI

SEQ ID NO: 193 - protein, 11 aa, Artificial Sequence
  Cadherin antagonist containing HAV binding motif
WLAMDPDSGQV

SEQ ID NO: 194 - protein, 11 aa, Artificial Sequence
  Cadherin antagonist containing HAV binding motif
WLHINATNGQI

SEQ ID NO: 195 - protein, 11 aa, Artificial Sequence
  Cadherin antagonist containing HAV binding motif
WLEINPDTGAI

SEQ ID NO: 196 - protein, 11 aa, Artificial Sequence
  Cadherin antagonist containing HAV binding motif
WLAVDPDSGQI

SEQ ID NO: 197 - protein, 11 aa, Artificial Sequence
  Cadherin antagonist containing HAV binding motif
WLEINPETGAI

SEQ ID NO: 198 - protein, 11 aa, Artificial Sequence
  Cadherin antagonist containing HAV binding motif
WLHINTSNGQI

SEQ ID NO: 199 - protein, 11 aa, Artificial Sequence
  Cadherin antagonist containing HAV binding motif
NLKIDPVNGQI

SEQ ID NO: 200 - protein, 10 aa, Artificial Sequence
  Cadherin antagonist containing HAV binding motif
LKIDPVNGQI

SEQ ID NO: 201 - protein, 7 aa, Artificial Sequence
  Cadherin antagonist containing HAV binding motif
INPISGQ

SEQ ID NO: 202 - protein, 7 aa, Artificial Sequence
  Cadherin antagonist containing HAV binding motif
LNPISGQ

SEQ ID NO: 203 - protein, 7 aa, Artificial Sequence
  Cadherin antagonist containing HAV binding motif
IDPVSGQ

SEQ ID NO: 204 - protein, 8 aa, Artificial Sequence
  Cadherin antagonist containing HAV binding motif
KIDPVNGQ

SEQ ID NO: 205 - protein, 5 aa, Artificial Sequence
  Cadherin antagonist containing HAV binding motif
PISGQ

SEQ ID NO: 206 - protein, 5 aa, Artificial Sequence
  Cadherin antagonist containing HAV binding motif
PVNGQ

SEQ ID NO: 207 - protein, 5 aa, Artificial Sequence
  Cadherin antagonist containing HAV binding motif
PVSGR

SEQ ID NO: 208 - protein, 5 aa, Artificial Sequence
  Cadherin antagonist containing HAV binding motif
IDPVN

SEQ ID NO: 209 - protein, 5 aa, Artificial Sequence
  Cadherin antagonist containing HAV binding motif
INPIS

SEQ ID NO: 210 - protein, 5 aa, Artificial Sequence
  Cadherin antagonist containing HAV binding motif
KIDPV

SEQ ID NO: 211 - protein, 9 aa, Artificial Sequence
  non-classical cadherin cell adhesion recognition sequence
XFXXXXXXG

SEQ ID NO: 212 - protein, 4 aa, Artificial Sequence
  OB-cadherin antagonist
IDDK

SEQ ID NO: 213 - protein, 4 aa, Artificial Sequence
  OB-cadherin antagonist
DDKS

SEQ ID NO: 214 - protein, 5 aa, Artificial Sequence
  OB-cadherin antagonist
VIDDK

SEQ ID NO: 215 - protein, 5 aa, Artificial Sequence
  OB-cadherin antagonist
IDDKS

SEQ ID NO: 216 - protein, 6 aa, Artificial Sequence
  OB-cadherin antagonist
VIDDKS

SEQ ID NO: 217 - protein, 5 aa, Artificial Sequence
  OB-cadherin antagonist
DDKSG

SEQ ID NO: 218 - protein, 6 aa, Artificial Sequence
  OB-cadherin antagonist
IDDKSG

SEQ ID NO: 219 - protein, 7 aa, Artificial Sequence
  OB-cadherin antagonist
VIDDKSG

SEQ ID NO: 220 - protein, 6 aa, Artificial Sequence
  OB-cadherin antagonist
FVIDDK

SEQ ID NO: 221 - protein, 7 aa, Artificial Sequence
  OB-cadherin antagonist
FVIDDKS

SEQ ID NO: 222 - protein, 8 aa, Artificial Sequence
  OB-cadherin antagonist
FVIDDKSG

SEQ ID NO: 223 - protein, 7 aa, Artificial Sequence
  OB-cadherin antagonist
IFVIDDK

SEQ ID NO: 224 - protein, 8 aa, Artificial Sequence
  OB-cadherin antagonist
IFVIDDKS

SEQ ID NO: 225 - protein, 9 aa, Artificial Sequence
  OB-cadherin antagonist
IFVIDDKSG

SEQ ID NO: 226 - protein, 4 aa, Artificial Sequence
  OB-cadherin antagonist
IEEY

SEQ ID NO: 227 - protein, 4 aa, Artificial Sequence
  OB-cadherin antagonist
EEYT

SEQ ID NO: 228 - protein, 5 aa, Artificial Sequence
  OB-cadherin antagonist
VIEEY

SEQ ID NO: 229 - protein, 5 aa, Artificial Sequence
  OB-cadherin antagonist
IEEYT

SEQ ID NO: 230 - protein, 6 aa, Artificial Sequence
  OB-cadherin antagonist
VIEEYT

SEQ ID NO: 231 - protein, 5 aa, Artificial Sequence
  OB-cadherin antagonist
EEYTG

SEQ ID NO: 232 - protein, 6 aa, Artificial Sequence
  OB-cadherin antagonist
IEEYTG

SEQ ID NO: 233 - protein, 7 aa, Artificial Sequence
  OB-cadherin antagonist
VIEEYTG

SEQ ID NO: 234 - protein, 6 aa, Artificial Sequence
  OB-cadherin antagonist
FVIEEY

SEQ ID NO: 235 - protein, 7 aa, Artificial Sequence
  OB-cadherin antagonist
FVIEEYT

SEQ ID NO: 236 - protein, 8 aa, Artificial Sequence
  OB-cadherin antagonist
FVIEEYTG

SEQ ID NO: 237 - protein, 7 aa, Artificial Sequence
  OB-cadherin antagonist
FFVIEEY

SEQ ID NO: 238 - protein, 8 aa, Artificial Sequence
  OB-cadherin antagonist
FFVIEEYT

SEQ ID NO: 239 - protein, 9 aa, Artificial Sequence
  OB-cadherin antagonist
FFVIEEYTG

SEQ ID NO: 240 - protein, 4 aa, Artificial Sequence
  OB-cadherin antagonist
VEAQ

SEQ ID NO: 241 - protein, 4 aa, Artificial Sequence
  OB-cadherin antagonist
EAQT

SEQ ID NO: 242 - protein, 5 aa, Artificial Sequence
  OB-cadherin antagonist
SVEAQ

SEQ ID NO: 243 - protein, 5 aa, Artificial Sequence
  OB-cadherin antagonist
VEAQT

SEQ ID NO: 244 - protein, 6 aa, Artificial Sequence
  OB-cadherin antagonist
SVEAQT

SEQ ID NO: 245 - protein, 5 aa, Artificial Sequence
  OB-cadherin antagonist
EAQTG

SEQ ID NO: 246 - protein, 6 aa, Artificial Sequence
  OB-cadherin antagonist
VEAQTG

SEQ ID NO: 247 - protein, 7 aa, Artificial Sequence
  OB-cadherin antagonist
SVEAQTG

SEQ ID NO: 248 - protein, 6 aa, Artificial Sequence
  OB-cadherin antagonist
FSVEAQ

SEQ ID NO: 249 - protein, 7 aa, Artificial Sequence
  OB-cadherin antagonist
FSVEAQT

SEQ ID NO: 250 - protein, 8 aa, Artificial Sequence
  OB-cadherin antagonist
FSVEAQTG

SEQ ID NO: 251 - protein, 7 aa, Artificial Sequence
  OB-cadherin antagonist
YFSVEAQ

SEQ ID NO: 252 - protein, 8 aa, Artificial Sequence
  OB-cadherin antagonist
YFSVEAQT

SEQ ID NO: 253 - protein, 9 aa, Artificial Sequence
  OB-cadherin antagonist
YFSVEAQTG

SEQ ID NO: 254 - protein, 4 aa, Artificial Sequence
  VE-cadherin antagonist
VDAE

SEQ ID NO: 255 - protein, 4 aa, Artificial Sequence
  VE-cadherin antagonist
DAET

SEQ ID NO: 256 - protein, 5 aa, Artificial Sequence
  VE-cadherin antagonist
RVDAE

SEQ ID NO: 257 - protein, 5 aa, Artificial Sequence
  VE-cadherin antagonist
VDAET

SEQ ID NO: 258 - protein, 6 aa, Artificial Sequence
  VE-cadherin antagonist
RVDAET

SEQ ID NO: 259 - protein, 5 aa, Artificial Sequence
  VE-cadherin antagonist
DAETG

SEQ ID NO: 260 - protein, 6 aa, Artificial Sequence
  VE-cadherin antagonist
VDAETG

SEQ ID NO: 261 - protein, 7 aa, Artificial Sequence
  VE-cadherin antagonist
RVDAETG

SEQ ID NO: 262 - protein, 6 aa, Artificial Sequence
  VE-cadherin antagonist
FRVDAE

SEQ ID NO: 263 - protein, 7 aa, Artificial Sequence
  VE-cadherin antagonist
FRVDAET

SEQ ID NO: 264 - protein, 8 aa, Artificial Sequence
  VE-cadherin antagonist
FRVDAETG

SEQ ID NO: 265 - protein, 7 aa, Artificial Sequence
  VE-cadherin antagonist
VFRVDAE

SEQ ID NO: 266 - protein, 8 aa, Artificial Sequence
  VE-cadherin antagonist
VFRVDAET

SEQ ID NO: 267 - protein, 9 aa, Artificial Sequence
  VE-cadherin antagonist
VFRVDAETG

SEQ ID NO: 268 - protein, 6 aa, Artificial Sequence
  Atypical cadherin Trp-containing cell adhesion recognition sequence
XWXWNQ

SEQ ID NO: 269 - protein, 4 aa, Artificial Sequence
  Atypical cadherin Trp-containing cell adhesion recognition sequence
GWVW

SEQ ID NO: 270 - protein, 5 aa, Artificial Sequence
  Atypical cadherin Trp-containing cell adhesion recognition sequence
GWVWN

SEQ ID NO: 271 - protein, 6 aa, Artificial Sequence
  Atypical cadherin Trp-containing cell adhesion recognition sequence
GWVWNQ

SEQ ID NO: 272 - protein, 4 aa, Artificial Sequence
  Atypical cadherin Trp-containing cell adhesion recognition sequence
WVWN

SEQ ID NO: 273 - protein, 5 aa, Artificial Sequence
  Atypical cadherin Trp-containing cell adhesion recognition sequence
WVWNQ

SEQ ID NO: 274 - protein, 4 aa, Artificial Sequence
  Atypical cadherin Trp-containing cell adhesion recognition sequence
DWIW

SEQ ID NO: 275 - protein, 5 aa, Artificial Sequence
  Atypical cadherin Trp-containing cell adhesion recognition sequence
DWIWN

SEQ ID NO: 276 - protein, 6 aa, Artificial Sequence
  Atypical cadherin Trp-containing cell adhesion recognition sequence
DWIWNQ

SEQ ID NO: 277 - protein, 4 aa, Artificial Sequence
  Atypical cadherin Trp-containing cell adhesion recognition sequence
WIWN

SEQ ID NO: 278 - protein, 5 aa, Artificial Sequence
  Atypical cadherin Trp-containing cell adhesion recognition sequence
WIWNQ

SEQ ID NO: 279 - protein, 7 aa, Artificial Sequence
  Atypical cadherin Trp-containing cell adhesion recognition sequence
SWMSWMW

SEQ ID NO: 280 - protein, 5 aa, Artificial Sequence
  Atypical cadherin Trp-containing cell adhesion recognition sequence
SWMWN

SEQ ID NO: 281 - protein, 6 aa, Artificial Sequence
  Atypical cadherin Trp-containing cell adhesion recognition sequence
SWMWNQ

SEQ ID NO: 282 - protein, 4 aa, Artificial Sequence
  Atypical cadherin Trp-containing cell adhesion recognition sequence
WMWN

SEQ ID NO: 283 - protein, 5 aa, Artificial Sequence
  Atypical cadherin Trp-containing cell adhesion recognition sequence
WMWNQ

SEQ ID NO: 284 - protein, 4 aa, Artificial Sequence
  Atypical cadherin Trp-containing cell adhesion recognition sequence
SWVW

SEQ ID NO: 285 - protein, 5 aa, Artificial Sequence
  Atypical cadherin Trp-containing cell adhesion recognition sequence
SWVWN

SEQ ID NO: 286 - protein, 6 aa, Artificial Sequence
  Atypical cadherin Trp-containing cell adhesion recognition sequence
SWVWNQ

SEQ ID NO: 287 - protein, 4 aa, Artificial Sequence
  Atypical cadherin Trp-containing cell adhesion recognition sequence
GWMW

SEQ ID NO: 288 - protein, 5 aa, Artificial Sequence
  Atypical cadherin Trp-containing cell adhesion recognition sequence
GWMWN

SEQ ID NO: 289 - protein, 6 aa, Artificial Sequence
  Atypical cadherin Trp-containing cell adhesion recognition sequence
GWMWNQ

SEQ ID NO: 290 - protein, 4 aa, Artificial Sequence
  Atypical cadherin Trp-containing cell adhesion recognition sequence
AWVI

SEQ ID NO: 291 - protein, 5 aa, Artificial Sequence
  Atypical cadherin Trp-containing cell adhesion recognition sequence
AWVIP

SEQ ID NO: 292 - protein, 6 aa, Artificial Sequence
  Atypical cadherin Trp-containing cell adhesion recognition sequence
AWVIPP

SEQ ID NO: 293 - protein, 4 aa, Artificial Sequence
  Atypical cadherin Trp-containing cell adhesion recognition sequence
WVIP

SEQ ID NO: 294 - protein, 5 aa, Artificial Sequence
  Atypical cadherin Trp-containing cell adhesion recognition sequence
WVIPP

SEQ ID NO: 295 - protein, 7 aa, Artificial Sequence
  Atypical cadherin Trp-containing cell adhesion recognition sequence
GWVWNQF

SEQ ID NO: 296 - protein, 8 aa, Artificial Sequence
  Atypical cadherin Trp-containing cell adhesion recognition sequence
GWVWNQFF

SEQ ID NO: 297 - protein, 9 aa, Artificial Sequence
  Atypical cadherin Trp-containing cell adhesion recognition sequence
GWVWNQFFV

SEQ ID NO: 298 - protein, 6 aa, Artificial Sequence
  Atypical cadherin Trp-containing cell adhesion recognition sequence
WVWNQF

SEQ ID NO: 299 - protein, 7 aa, Artificial Sequence
  Atypical cadherin Trp-containing cell adhesion recognition sequence
WVWNQFF

SEQ ID NO: 300 - protein, 8 aa, Artificial Sequence
  Atypical cadherin Trp-containing cell adhesion recognition sequence
WVWNQFFV

SEQ ID NO: 301 - protein, 4 aa, Artificial Sequence
  Atypical cadherin Trp-containing cell adhesion recognition sequence
RGWV

SEQ ID NO: 302 - protein, 5 aa, Artificial Sequence
  Atypical cadherin Trp-containing cell adhesion recognition sequence
RGWVW

SEQ ID NO: 303 - protein, 6 aa, Artificial Sequence
  Atypical cadherin Trp-containing cell adhesion recognition sequence
RGWVWN

SEQ ID NO: 304 - protein, 7 aa, Artificial Sequence
  Atypical cadherin Trp-containing cell adhesion recognition sequence
RGWVWNQ

SEQ ID NO: 305 - protein, 8 aa, Artificial Sequence
  Atypical cadherin Trp-containing cell adhesion recognition sequence
RGWVWNQF

SEQ ID NO: 306 - protein, 9 aa, Artificial Sequence
  Atypical cadherin Trp-containing cell adhesion recognition sequence
RGWVWNQFF

SEQ ID NO: 307 - protein, 10 aa, Artificial Sequence
  Atypical cadherin Trp-containing cell adhesion recognition sequence
RGWVWNQFFV

SEQ ID NO: 308 - protein, 4 aa, Artificial Sequence
  Atypical cadherin Trp-containing cell adhesion recognition sequence
KRGW

SEQ ID NO: 309 - protein, 5 aa, Artificial Sequence
  Atypical cadherin Trp-containing cell adhesion recognition sequence
KRGWV

SEQ ID NO: 310 - protein, 6 aa, Artificial Sequence
  Atypical cadherin Trp-containing cell adhesion recognition sequence
KRGWVW

SEQ ID NO: 311 - protein, 7 aa, Artificial Sequence
  Atypical cadherin Trp-containing cell adhesion recognition sequence
KRGWVWN

SEQ ID NO: 312 - protein, 8 aa, Artificial Sequence
  Atypical cadherin Trp-containing cell adhesion recognition sequence
KRGWVWNQ

SEQ ID NO: 313 - protein, 9 aa, Artificial Sequence
  Atypical cadherin Trp-containing cell adhesion recognition sequence
KRGWVWNQF

SEQ ID NO: 314 - protein, 10 aa, Artificial Sequence
  Atypical cadherin Trp-containing cell adhesion recognition sequence
KRGWVWNQFF

SEQ ID NO: 315 - protein, 11 aa, Artificial Sequence
  Atypical cadherin Trp-containing cell adhesion recognition sequence
KRGWVWNQFFV

SEQ ID NO: 316 - protein, 7 aa, Artificial Sequence
  Atypical cadherin Trp-containing cell adhesion recognition sequence
DWIWNQM

SEQ ID NO: 317 - protein, 8 aa, Artificial Sequence
  Atypical cadherin Trp-containing cell adhesion recognition sequence
DWIWNQMH

SEQ ID NO: 318 - protein, 9 aa, Artificial Sequence
  Atypical cadherin Trp-containing cell adhesion recognition sequence
DWIWNQMHI

SEQ ID NO: 319 - protein, 6 aa, Artificial Sequence
  Atypical cadherin Trp-containing cell adhesion recognition sequence
WIWNQM

SEQ ID NO: 320 - protein, 7 aa, Artificial Sequence
  Atypical cadherin Trp-containing cell adhesion recognition sequence
WIWNQMH

SEQ ID NO: 321 - protein, 8 aa, Artificial Sequence
  Atypical cadherin Trp-containing cell adhesion recognition sequence
WIWNQMHI

SEQ ID NO: 322 - protein, 4 aa, Artificial Sequence
  Atypical cadherin Trp-containing cell adhesion recognition sequence
RDWI

SEQ ID NO: 323 - protein, 5 aa, Artificial Sequence
  Atypical cadherin Trp-containing cell adhesion recognition sequence
RDWIW

SEQ ID NO: 324 - protein, 6 aa, Artificial Sequence
  Atypical cadherin Trp-containing cell adhesion recognition sequence
RDWIWN

SEQ ID NO: 325 - protein, 7 aa, Artificial Sequence
  Atypical cadherin Trp-containing cell adhesion recognition sequence
RDWIWNQ

SEQ ID NO: 326 - protein, 8 aa, Artificial Sequence
  Atypical cadherin Trp-containing cell adhesion recognition sequence
RDWIWNQM

SEQ ID NO: 327 - protein, 9 aa, Artificial Sequence
  Atypical cadherin Trp-containing cell adhesion recognition sequence
RDWIWNQMH

SEQ ID NO: 328 - protein, 10 aa, Artificial Sequence
  Atypical cadherin Trp-containing cell adhesion recognition sequence
RDWIWNQMHI

SEQ ID NO: 329 - protein, 4 aa, Artificial Sequence
  Atypical cadherin Trp-containing cell adhesion recognition sequence
KRDW

SEQ ID NO: 330 - protein, 5 aa, Artificial Sequence
  Atypical cadherin Trp-containing cell adhesion recognition sequence
KRDWI

SEQ ID NO: 331 - protein, 6 aa, Artificial Sequence
  Atypical cadherin Trp-containing cell adhesion recognition sequence
KRDWIW

SEQ ID NO: 332 - protein, 7 aa, Artificial Sequence
  Atypical cadherin Trp-containing cell adhesion recognition sequence
KRDWIWN

SEQ ID NO: 333 - protein, 8 aa, Artificial Sequence
  Atypical cadherin Trp-containing cell adhesion recognition sequence
KRDWIWNQ

SEQ ID NO: 334 - protein, 9 aa, Artificial Sequence
  Atypical cadherin Trp-containing cell adhesion recognition sequence
KRDWIWNQM

SEQ ID NO: 335 - protein, 10 aa, Artificial Sequence
  Atypical cadherin Trp-containing cell adhesion recognition sequence
KRDWIWNQMH

SEQ ID NO: 336 - protein, 11 aa, Artificial Sequence
  Atypical cadherin Trp-containing cell adhesion recognition sequence
KRDWIWNQMHI

SEQ ID NO: 337 - protein, 7 aa, Artificial Sequence
  Atypical cadherin Trp-containing cell adhesion recognition sequence
SWMWNQF

SEQ ID NO: 338 - protein, 8 aa, Artificial Sequence
  Atypical cadherin Trp-containing cell adhesion recognition sequence
SWMWNQFF

SEQ ID NO: 339 - protein, 9 aa, Artificial Sequence
  Atypical cadherin Trp-containing cell adhesion recognition sequence
SWMWNQFFL

SEQ ID NO: 340 - protein, 6 aa, Artificial Sequence
  Atypical cadherin Trp-containing cell adhesion recognition sequence
WMWNQF

SEQ ID NO: 341 - protein, 7 aa, Artificial Sequence
  Atypical cadherin Trp-containing cell adhesion recognition sequence
WMWNQFF

SEQ ID NO: 342 - protein, 8 aa, Artificial Sequence
  Atypical cadherin Trp-containing cell adhesion recognition sequence
WMWNQFFL

SEQ ID NO: 343 - protein, 4 aa, Artificial Sequence
  Atypical cadherin Trp-containing cell adhesion recognition sequence
RSWM

SEQ ID NO: 344 - protein, 5 aa, Artificial Sequence
  Atypical cadherin Trp-containing cell adhesion recognition sequence
RSWMW

SEQ ID NO: 345 - protein, 6 aa, Artificial Sequence
  Atypical cadherin Trp-containing cell adhesion recognition sequence
RSWMWN

SEQ ID NO: 346 - protein, 7 aa, Artificial Sequence
  Atypical cadherin Trp-containing cell adhesion recognition sequence
RSWMWNQ

SEQ ID NO: 347 - protein, 8 aa, Artificial Sequence
  Atypical cadherin Trp-containing cell adhesion recognition sequence
RSWMWNQF

SEQ ID NO: 348 - protein, 9 aa, Artificial Sequence
  Atypical cadherin Trp-containing cell adhesion recognition sequence
RSWMWNQFF

SEQ ID NO: 349 - protein, 10 aa, Artificial Sequence
  Atypical cadherin Trp-containing cell adhesion recognition sequence
RSWMWNQFFL

SEQ ID NO: 350 - protein, 4 aa, Artificial Sequence
  Atypical cadherin Trp-containing cell adhesion recognition sequence
KRSW

SEQ ID NO: 351 - protein, 5 aa, Artificial Sequence
  Atypical cadherin Trp-containing cell adhesion recognition sequence
KRSWM

SEQ ID NO: 352 - protein, 6 aa, Artificial Sequence
  Atypical cadherin Trp-containing cell adhesion recognition sequence
KRSWMW

SEQ ID NO: 353 - protein, 7 aa, Artificial Sequence
  Atypical cadherin Trp-containing cell adhesion recognition sequence
KRSWMWN

SEQ ID NO: 354 - protein, 8 aa, Artificial Sequence
  Atypical cadherin Trp-containing cell adhesion recognition sequence
KRSWMWNQ

SEQ ID NO: 355 - protein, 9 aa, Artificial Sequence
  Atypical cadherin Trp-containing cell adhesion recognition sequence
KRSWMWNQF

SEQ ID NO: 356 - protein, 10 aa, Artificial Sequence
  Atypical cadherin Trp-containing cell adhesion recognition sequence
KRSWMWNQFF

SEQ ID NO: 357 - protein, 11 aa, Artificial Sequence
  Atypical cadherin Trp-containing cell adhesion recognition sequence
KRSWMWNQFFL

SEQ ID NO: 358 - protein, 7 aa, Artificial Sequence
  Atypical cadherin Trp-containing cell adhesion recognition sequence
SWVWNQF

SEQ ID NO: 359 - protein, 8 aa, Artificial Sequence
  Atypical cadherin Trp-containing cell adhesion recognition sequence
SWVWNQFF

SEQ ID NO: 360 - protein, 9 aa, Artificial Sequence
  Atypical cadherin Trp-containing cell adhesion recognition sequence
SWVWNQFFV

SEQ ID NO: 361 - protein, 6 aa, Artificial Sequence
  Atypical cadherin Trp-containing cell adhesion recognition sequence
WVWNQF

SEQ ID NO: 362 - protein, 7 aa, Artificial Sequence
  Atypical cadherin Trp-containing cell adhesion recognition sequence
WVWNQFF

SEQ ID NO: 363 - protein, 8 aa, Artificial Sequence
  Atypical cadherin Trp-containing cell adhesion recognition sequence
WVWNQFFV

SEQ ID NO: 364 - protein, 4 aa, Artificial Sequence
  Atypical cadherin Trp-containing cell adhesion recognition sequence
RSWV

SEQ ID NO: 365 - protein, 5 aa, Artificial Sequence
  Atypical cadherin Trp-containing cell adhesion recognition sequence
RSWVW

SEQ ID NO: 366 - protein, 6 aa, Artificial Sequence
  Atypical cadherin Trp-containing cell adhesion recognition sequence
RSWVWN

SEQ ID NO: 367 - protein, 7 aa, Artificial Sequence
  Atypical cadherin Trp-containing cell adhesion recognition sequence
RSWVWNQ

SEQ ID NO: 368 - protein, 8 aa, Artificial Sequence
  Atypical cadherin Trp-containing cell adhesion recognition sequence
RSWVWNQF

SEQ ID NO: 369 - protein, 9 aa, Artificial Sequence
  Atypical cadherin Trp-containing cell adhesion recognition sequence
RSWVWNQFF

SEQ ID NO: 370 - protein, 10 aa, Artificial Sequence
  Atypical cadherin Trp-containing cell adhesion recognition sequence
RSWVWNQFFV

SEQ ID NO: 371 - protein, 5 aa, Artificial Sequence
  Atypical cadherin Trp-containing cell adhesion recognition sequence
KRSWV

SEQ ID NO: 372 - protein, 6 aa, Artificial Sequence
  Atypical cadherin Trp-containing cell adhesion recognition sequence
KRSWVW

SEQ ID NO: 373 - protein, 7 aa, Artificial Sequence
  Atypical cadherin Trp-containing cell adhesion recognition sequence
KRSWVWN

SEQ ID NO: 374 - protein, 8 aa, Artificial Sequence
  Atypical cadherin Trp-containing cell adhesion recognition sequence
KRSWVWNQ

SEQ ID NO: 375 - protein, 9 aa, Artificial Sequence
  Atypical cadherin Trp-containing cell adhesion recognition sequence
KRSWVWNQF

SEQ ID NO: 376 - protein, 10 aa, Artificial Sequence
  Atypical cadherin Trp-containing cell adhesion recognition sequence
KRSWVWNQFF

SEQ ID NO: 377 - protein, 11 aa, Artificial Sequence
  Atypical cadherin Trp-containing cell adhesion recognition sequence
KRSWVWNQFFV

SEQ ID NO: 378 - protein, 7 aa, Artificial Sequence
  Atypical cadherin Trp-containing cell adhesion recognition sequence
GWVWNQM

SEQ ID NO: 379 - protein, 8 aa, Artificial Sequence
  Atypical cadherin Trp-containing cell adhesion recognition sequence
GWVWNQMF

SEQ ID NO: 380 - protein, 9 aa, Artificial Sequence
  Atypical cadherin Trp-containing cell adhesion recognition sequence
GWVWNQMFV

SEQ ID NO: 381 - protein, 8 aa, Artificial Sequence
  Atypical cadherin Trp-containing cell adhesion recognition sequence
RGWVWNQM

SEQ ID NO: 382 - protein, 9 aa, Artificial Sequence
  Atypical cadherin Trp-containing cell adhesion recognition sequence
RGWVWNQMF

SEQ ID NO: 383 - protein, 10 aa, Artificial Sequence
  Atypical cadherin Trp-containing cell adhesion recognition sequence
RGWVWNQMFV

SEQ ID NO: 384 - protein, 9 aa, Artificial Sequence
  Atypical cadherin Trp-containing cell adhesion recognition sequence
KRGWVWNQM

SEQ ID NO: 385 - protein, 11 aa, Artificial Sequence
  Atypical cadherin Trp-containing cell adhesion recognition sequence
KRGWVWNQMFV

SEQ ID NO: 386 - protein, 9 aa, Artificial Sequence
  Atypical cadherin Trp-containing cell adhesion recognition sequence
GWVWNQFFL

SEQ ID NO: 387 - protein, 10 aa, Artificial Sequence
  Atypical cadherin Trp-containing cell adhesion recognition sequence
RGWVWNQFFL

SEQ ID NO: 388 - protein, 11 aa, Artificial Sequence
  Atypical cadherin Trp-containing cell adhesion recognition sequence
KRGWVWNQFFL

SEQ ID NO: 389 - protein, 7 aa, Artificial Sequence
  Atypical cadherin Trp-containing cell adhesion recognition sequence
AWVIPPI

SEQ ID NO: 390 - protein, 8 aa, Artificial Sequence
  Atypical cadherin Trp-containing cell adhesion recognition sequence
AWVIPPIS

SEQ ID NO: 391 - protein, 9 aa, Artificial Sequence
  Atypical cadherin Trp-containing cell adhesion recognition sequence
AWVIPPISV

SEQ ID NO: 392 - protein, 6 aa, Artificial Sequence
  Atypical cadherin Trp-containing cell adhesion recognition sequence
WVIPPI

SEQ ID NO: 393 - protein, 7 aa, Artificial Sequence
  Atypical cadherin Trp-containing cell adhesion recognition sequence
WVIPPIS

SEQ ID NO: 394 - protein, 8 aa, Artificial Sequence
  Atypical cadherin Trp-containing cell adhesion recognition sequence
WVIPPISV

SEQ ID NO: 395 - protein, 4 aa, Artificial Sequence
  Atypical cadherin Trp-containing cell adhesion recognition sequence
RAWV

SEQ ID NO: 396 - protein, 5 aa, Artificial Sequence
  Atypical cadherin Trp-containing cell adhesion recognition sequence
RAWVI

SEQ ID NO: 397 - protein, 6 aa, Artificial Sequence
  Atypical cadherin Trp-containing cell adhesion recognition sequence
RAWVIP

SEQ ID NO: 398 - protein, 7 aa, Artificial Sequence
  Atypical cadherin Trp-containing cell adhesion recognition sequence
RAWVIPP

SEQ ID NO: 399 - protein, 8 aa, Artificial Sequence
  Atypical cadherin Trp-containing cell adhesion recognition sequence
RAWVIPPI

SEQ ID NO: 400 - protein, 9 aa, Artificial Sequence
  Atypical cadherin Trp-containing cell adhesion recognition sequence
RAWVIPPIS

SEQ ID NO: 401 - protein, 10 aa, Artificial Sequence
  Atypical cadherin Trp-containing cell adhesion recognition sequence
RAWVIPPISV

SEQ ID NO: 402 - protein, 4 aa, Artificial Sequence
  Atypical cadherin Trp-containing cell adhesion recognition sequence
KRAW

SEQ ID NO: 403 - protein, 5 aa, Artificial Sequence
  Atypical cadherin Trp-containing cell adhesion recognition sequence
KRAWV

SEQ ID NO: 404 - protein, 6 aa, Artificial Sequence
  Atypical cadherin Trp-containing cell adhesion recognition sequence
KRAWVI

SEQ ID NO: 405 - protein, 7 aa, Artificial Sequence
  Atypical cadherin Trp-containing cell adhesion recognition sequence
KRAWVIP

SEQ ID NO: 406 - protein, 8 aa, Artificial Sequence
  Atypical cadherin Trp-containing cell adhesion recognition sequence
KRAWVIPP

SEQ ID NO: 407 - protein, 9 aa, Artificial Sequence
  Atypical cadherin Trp-containing cell adhesion recognition sequence
KRAWVIPPI

SEQ ID NO: 408 - protein, 10 aa, Artificial Sequence
  Atypical cadherin Trp-containing cell adhesion recognition sequence
KRAWVIPPIS

SEQ ID NO: 409 - protein, 4 aa, Artificial Sequence
  Atypical cadherin Trp-containing cell adhesion recognition sequence
VWNQ

SEQ ID NO: 410 - protein, 5 aa, Artificial Sequence
  Atypical cadherin Trp-containing cell adhesion recognition sequence
VWNQM

SEQ ID NO: 411 - protein, 5 aa, Artificial Sequence
  Atypical cadherin Trp-containing cell adhesion recognition sequence
VWNQF

SEQ ID NO: 412 - protein, 6 aa, Artificial Sequence
  Atypical cadherin Trp-containing cell adhesion recognition sequence
VWNQMF

SEQ ID NO: 413 - protein, 6 aa, Artificial Sequence
  Atypical cadherin Trp-containing cell adhesion recognition sequence
VWNQFF

SEQ ID NO: 414 - protein, 4 aa, Artificial Sequence
  Atypical cadherin Trp-containing cell adhesion recognition sequence
WNQM

SEQ ID NO: 415 - protein, 4 aa, Artificial Sequence
  Atypical cadherin Trp-containing cell adhesion recognition sequence
WNQF

SEQ ID NO: 416 - protein, 5 aa, Artificial Sequence
  Atypical cadherin Trp-containing cell adhesion recognition sequence
WNQFF

SEQ ID NO: 417 - protein, 4 aa, Artificial Sequence
  Atypical cadherin Trp-containing cell adhesion recognition sequence
IWNQ

SEQ ID NO: 418 - protein, 5 aa, Artificial Sequence
  Atypical cadherin Trp-containing cell adhesion recognition sequence
IWNQM

SEQ ID NO: 419 - protein, 6 aa, Artificial Sequence
  Atypical cadherin Trp-containing cell adhesion recognition sequence
IWNQMH

SEQ ID NO: 420 - protein, 4 aa, Artificial Sequence
  Atypical cadherin Trp-containing cell adhesion recognition sequence
WNQM

SEQ ID NO: 421 - protein, 5 aa, Artificial Sequence
  Atypical cadherin Trp-containing cell adhesion recognition sequence
WNQMH

SEQ ID NO: 422 - protein, 4 aa, Artificial Sequence
  Atypical cadherin Trp-containing cell adhesion recognition sequence
MWNQ

SEQ ID NO: 423 - protein, 5 aa, Artificial Sequence
  Atypical cadherin Trp-containing cell adhesion recognition sequence
MWNQF

SEQ ID NO: 424 - protein, 6 aa, Artificial Sequence
  Atypical cadherin Trp-containing cell adhesion recognition sequence
MWNQFF

(19) United States
(12) Patent Application Publication
(10) Pub. No.: US 2009/0131306 A1
(43) Pub. Date: May 21, 2009

(54) CHEMICALLY MODIFIED CYCLIC PEPTIDES CONTAINING CELL ADHESION RECOGNITION (CAR) SEQUENCES AND USES THEREFOR

(75) Inventors: Mukur Gupta, Morrisville, NC (US); Brian Huber, Durham, NC (US); William Paul Peters, Fernandina Beach, FL (US)

Correspondence Address: SEED INTELLECTUAL PROPERTY LAW GROUP PLLC, 701 FIFTH AVE, SUITE 5400, SEATTLE, WA, 98104, US

(73) Assignee: ADHEREX TECHNOLOGIES, INC., Ottawa (CA)

(21) Appl. No.: 12/139,270

(22) Filed: Jun. 13, 2008

Related U.S. Application Data
(60) Provisional application No. 60/944,419, filed on Jun. 15, 2007.

(51) Int. Cl. A61K 38/16; C07K 14/00; A61P 35/00

(52) U.S. Cl. 514/11; 530/317

(57) ABSTRACT

Chemically modified cyclic peptides comprising cell adhesion recognition (CAR) sequences are provided having improved properties, such as improved stability. Also provided are methods of making and using same.

### BACKGROUND

1. Field of the Invention

The present invention relates generally to chemically modified disulfide-containing cyclic peptides offering improved properties, such as activity and/or in vivo stability.

2. Description of the Related Art

Peptides have been effectively used as drug molecules. For example, there are over 50 peptide drugs on the market or in development today, including cytokines (interferon-gamma, interferon-alpha, interleukins, TNF, GM-CSF, G-CSF, etc), somatotropin, insulin, growth hormone, glucagon, gonadotrophins, LHRH, inhibin, erythropoietin, thyrotrophin, ACTH, prolactin, melanocyte stimulating hormone, therapeutic enzymes and cell adhesion factors, in addition to many peptide based vaccines.

Although effective, peptides have significant limitations in terms of efficacy and compliance which limit their use. In general, issues which affect the overall effectiveness of peptides and proteins as drugs include molecular size, susceptibility to proteolytic breakdown, rapid plasma clearance, tendency to undergo aggregation and immunogenicity. In general, peptides are not orally active due to breakdown in the GI tract, although there are exceptions. Most peptides are administered intravenously or intra-arterially, although others routes of administration have been used, including buccal, rectal, nasal, vaginal, and transdermal. In addition, linear chain peptides are subject to proteases and peptidases that are found in the circulation and in tissues. Degradation by peptidases and proteases will limit the exposure of the peptide drug due to breakdown. Another limitation of peptides is that they can be immunogenic, even if they are human in origin. Immunogenicity can not only arise from differences in primary, secondary and tertiary structure of the administered peptide compared to its natural homologue, but also arise from covalent or ionic interactions with other natural peptides, thereby acting as a hapten and creating an immunogenic complex.

Innovative approaches have tried to address these general limitations of peptides. Pegylation of peptide molecules have generated peptides that have relatively long depot half-lives and may be less immunogenic in certain circumstances. However, pegylating has been noted to decrease potency and efficacy in certain situations, probably as the result of masking the active site on the peptide or protein.

Cyclization has been another method to address the limitations of peptides. As example Arg-Gly-Asp (RGD) peptides contain an aspartic acid residue that is highly susceptible to chemical degradation. It has been shown that cyclization of this peptide by disulphide bond linkage can induce structural rigidity and prevents rapid degradation. Hence cyclization of small peptides may help in serum stability. Although disulfide cyclization makes peptides more stable, in vivo serum half-lives are still very short, measured in minutes and are very unstable above pH 8.

The cyclic peptide referred to as ADH-1, comprising the classical cadherin cell adhesion recognition (CAR) sequence His-Ala-Val, is currently under clinical study as an anti-cancer agent by virtue of its ability to interrupt cell adhesion mediated by cadherin receptors. The structure of ADH-1 is shown in FIG. 1.

One limitation associated with ADH-1 is the relatively short half life observed following i.v. administration. The circulating half-life of ADH-1 in humans is 2.5 hours with 30% excreted unchanged in the urine. Several possible reasons for this short half-life exist, however the lability of the disulfide bond is likely a significant factor. Disulfides are known to be susceptible to cleavage via reductive mechanisms and also by disulfide exchange mechanisms. These events can generate intermediates that can be further conjugated via normal metabolism processes and the resulting compounds are usually excreted rapidly.

There is thus a significant need for identifying modifications to disulfide-containing cyclic peptides, such as ADH-1 and the many other CAR-containing cyclic peptides known in the art, that result in more stable compounds in blood and increase circulating half-life, exposure and/or clinical activity. The present invention fulfills these needs and offers other related advantages.

### SUMMARY OF THE INVENTION

According to one aspect of the invention, there is provided a cyclic peptide or salt thereof that comprises an intramolecular covalent disulfide bond between two non-adjacent residues and at least one cell adhesion recognition (CAR) sequence; wherein the disulfide bond has been modified as set forth in structures (A)-(E) below:

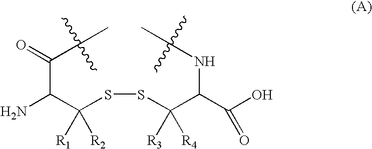

wherein R₁, R₂, R₃ and R₄ are the same or different and independently selected from H, alkyl, alkenyl, alkynyl, cycloalkyl, cycloalkyl-alkyl, heterocyclyl, heterocyclyl-alkyl, aryl, alkylaryl, arylalkyl, heteroaryl, alkylheteroaryl, heteroarylalkyl, alkoxy, alkylalkoxy, alkylthio, alkylamino, carbocycle, substituted carbocycle, heterocycle, substituted heterocycle, or —(CH₂)ₙ—X—(CH₂)ₘ— where n and m are independently 1, 2, 3 or 4 and X═O, S, NH, or N-alkyl;

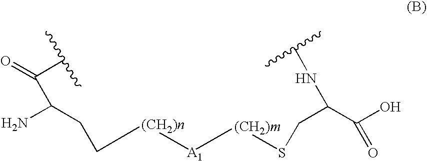

wherein n and m are the same or different and independently 1, 2, 3 or 4 and A₁ is aryl, substituted aryl, heteraryl, substituted heteroaryl, carbonyl, a carbon bearing an alcohol, a carbon bearing an alkylether, a carbon bearing an arylether, a carbon bearing an heteroarylether, a carbon bearing an alkylester, a carbon bearing an arylester, a carbon bearing an heteroarylester, a carbon bearing an amine, a carbon bearing an alkylamine, a carbon bearing an arylamine, a carbon bearing a heteroarylamine, a carbon bearing an amide, or a carbon bearing an alkylamide;

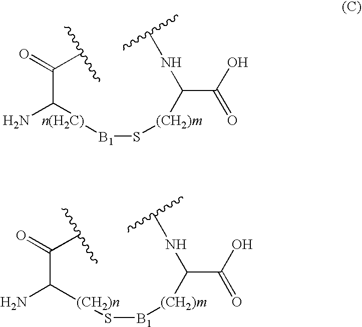

wherein n and m are independently 1, 2, 3 or 4; and B₂ is aryl or heteroaryl;

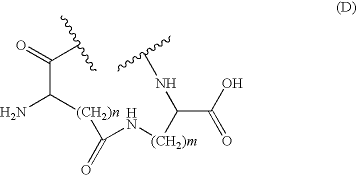

wherein n and m are independently 1, 2, 3 or 4; or

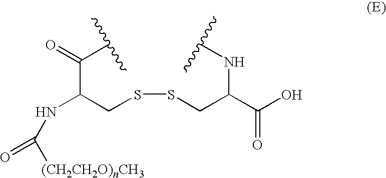

wherein n is in a range from about 1 to 100, and wherein the cyclic peptide is optionally acetylated at the N-terminus and/or amidated at the C-terminus.

In one embodiment, the size of a cyclic peptide ring generally ranges from 5 to about 15 residues, preferably from 5 to 10 residues.

In another embodiment, the modified disulfide bond has a structure selected from:

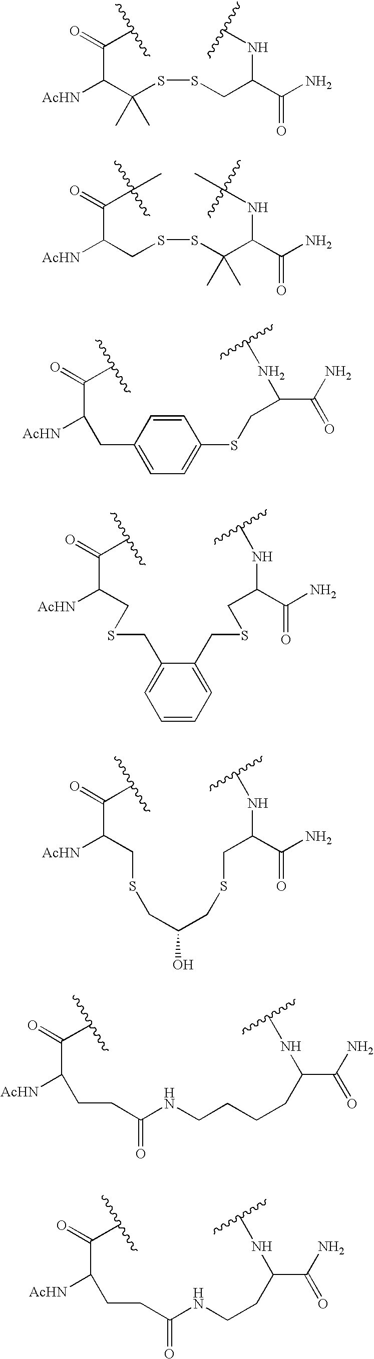

In a more particular embodiment, the CAR sequence comprises the sequence HAV. In another particular embodiment, the CAR comprises the sequence HAV and the cyclic peptide has the following formula:

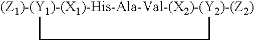

wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; and wherein a covalent disulfide bond is formed between residues Y₁ and Y₂ which is modified as set forth in any one of (A)-(E) of claim 1, and wherein Z₁ and Z₂ are optional, and if present, are independently selected from the group consisting of amino acid residues and combinations thereof in which the residues are linked by peptide bonds.

In yet another embodiment, the CAR comprises the sequence: Asp/Glu-Trp-Val-Ile/Val/Met-Pro/Ala-Pro (SEQ ID NO:40), wherein “Asp/Glu” is an amino acid that is either Asp or Glu, “Ile/Val/Met” is an amino acid that is Ile, Val or Met, and “Pro/Ala” is either Pro or Ala.

In another embodiment, the CAR comprises the sequence: Gly/Asp/Ser-Trp-Val/Ile/Met-Trp-Asn-Gln (SEQ ID NO: 268), wherein “Gly/Asp/Ser” is an amino acid that is Gly, Asp or Ser; and “Val/Ile/Met” is an amino acid that is Val, Ile or Met.

In still another embodiment, the CAR comprises the sequence: (a) Ile/Val-Phe-Aaa-Ile-Baa-Caa-Daa-Ser/Thr-Gly-Eaa-Leu/Met (SEQ ID NO:182), wherein Aaa, Baa, Caa, Daa and Eaa are independently selected from the group consisting of amino acid residues; or comprises the sequence Trp-Leu-Aaa-Ile-Asp/Asn-Baa-Caa-Daa-Gly-Gln-Ile (SEQ ID NO: 183), wherein Aaa, Baa, Caa and Daa are independently selected from the group consisting of amino acid residues.

In another embodiment, the CAR comprises the sequence: Aaa-Phe-Baa-Ile/Leu/Val-Asp/Asn/Glu-Caa-Daa-Ser/Thr/Asn-Gly (SEQ ID NO:211) wherein Aaa, Baa, Caa and Daa are independently selected from amino acid residues; Ile/Leu/Val is an amino acid that is selected from the group consisting of isoleucine, leucine and valine, Asp/Asn/Glu is an amino acid that is selected from the group consisting of aspartate, asparagine and glutamate; and Ser/Thr/Asn is an amino acid that is selected from the group consisting of serine, threonine or asparagine.

In another aspect of the invention, pharmaceutical compositions are provided comprising a cyclic peptide as described herein, in combination with a physiologically acceptable carrier.

In another aspect, the invention provides pharmaceutical compositions comprising a cyclic peptide, as described herein, in combination with a physiologically acceptable carrier, and further comprising one or more anticancer agents. In a particular embodiment, the one or more anticancer agents are selected from the group consisting of alkylating agents, antimetabolites, natural products, antibiotics, platinum compounds, anthracenediones, methylhydrazine derivatives, adrenocortical suppressants, tyrosine kinase inhibitors, multi-targeted kinase inhibitors, adrenocorticosteroids, estrogens, progestins, aromatase inhibitors, antiestrogens, antitumor antibodies and radiation therapy.

According to another aspect of the invention, there is provided a method for treating a condition or disease associated with altered cell adhesion, such as cancer, comprising administering to a subject in need thereof a pharmaceutical composition according as described herein.

Also provided by the invention are methods for improving the stability of a disulfide bond-containing cyclic peptide comprising introducing one or more chemical modifications as described herein.

Further still, the invention provides antibodies and antigen-binding fragments thereof that specifically bind to a modified cyclic peptide described herein, but preferably do not specifically bind to the corresponding unmodified cyclic peptide.

### BRIEF DESCRIPTION OF THE DRAWINGS

FIG. 1 shows the chemical structure of the cyclic peptide referred to as ADH-1 (N-Ac-CHAVC-NH2).

FIG. 2A shows the chemical structure of the modified ADH-1 compound referred to as ADH100701-T, in which the disulfide bond has been oxidized to form a thiosulfonate. FIG. 2B shows the results of stability testing in PBS and whole blood.

FIGS. 3A-3D show the chemical structures of modified ADH-1 compounds in which steric R groups were introduced adjacent to sulfur atoms of the disulfide bond. FIG. 3E shows the results of stability testing in PBS and whole blood.

FIG. 4A shows the chemical structures of the modified ADH-1 compound, referred to as ADH100706-T, in which the disulfide bond was modified by dithiolalkylation with an acetone moiety. FIG. 4B shows the results of stability testing in PBS and whole blood.

FIGS. 5A-B show the chemical structures of the modified ADH-1 compounds, referred to as ADH100710-T and ADH100705-T, in which the disulfide bond was modified by dithiolalkylation with an aromatic moiety. FIG. 5C shows the results of stability testing in PBS and whole blood.

FIGS. 6A-B show the chemical structure and results of stability testing for the modified ADH-1 compound, referred to as ADH100712-T, in which the disulfide bond was replaced with an alcohol derivative. FIGS. 6C-D show the chemical structure and results of stability testing for the modified ADH-1 compound, referred to as ADH100713-T, in which the disulfide bond was replaced with an alcohol derivative.

FIG. 7A shows the chemical structure of the modified ADH-1 compound, referred to as ADH100704-T, in which one of the sulfur atoms of the disulfide bond was replaced with a simple thioether linkage. FIG. 7B shows the results of stability testing in PBS and whole blood.

FIG. 8A shows the chemical structure of the modified ADH-1 compound, referred to as ADH100716-T, in which one of the sulfur atoms of the disulfide bond was replaced with an aromatic thioether linkage. FIG. 8B shows the results of stability testing in PBS and whole blood.

FIGS. 9A-B show the chemical structures of modified ADH-1 compounds, in which both sulfur atoms of the disulfide bond are replaced with an amide derivative. FIG. 9C shows the results of stability testing in PBS and whole blood for the modified compound referred to as ADH100714-T.

FIG. 10A shows the chemical structure of the modified ADH-1 compound referred to as ADH100707-T, in which the N-acetyl group has been modified by additional of polyethylene glycol (PEG). FIG. 10B shows the results of stability testing in PBS and whole blood.

### DETAILED DESCRIPTION OF THE INVENTION

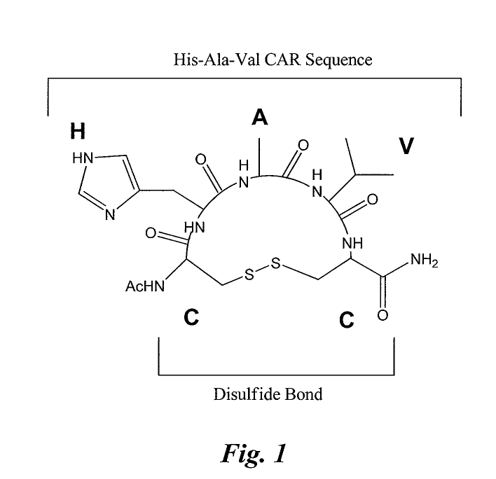

The present invention relates generally to chemical modifications effective for stabilizing disulfide-containing cyclic peptides, such as ADH-1, from physiochemical and/or enzymatic degradation in biological fluids. Stabilization of the disulfide bond was approached using several different methods, including direct modification of the disulfide bond, replacement of disulfide atoms and/or introduction of additional chemical constituents in the vicinity of the disulfide bond. As described herein, particular classes of chemical modifications have been identified that result in unexpected and unpredictable improvements in their stability in biological fluids, while other modifications resulted in little or no improvements. For example, while ADH-1 (N-Ac-CHAVC-NH2; detailed chemical structure shown in FIG. 1) has a half-life in human blood of only about 2 hours, ADH-1 compounds having modifications as described herein have been demonstrated to have half-lives in human blood of greater than 12 hours. Furthermore, based on the structural similarities of cyclic peptides comprising the CAR sequence HAV (such as ADH-1) and cyclic peptides comprising other known CAR sequences, the modifications described herein can provide improved stability for a broad range of CAR-containing cyclic peptides.

Therefore, according to a general aspect of the invention, there are provided cyclic peptides comprising at least one cell adhesion recognition (CAR) sequence, as described herein, wherein the cyclic peptide is cyclized via a modified disulfide bond to improve stability in biological fluids, particularly human blood.

The term “cyclic peptide” refers to a peptide or salt thereof that comprises (1) an intramolecular covalent disulfide bond between two non-adjacent residues and (2) at least one cell adhesion recognition (CAR) sequence; wherein the disulfide bond has been modified in order to improve the stability of the cyclic peptide in biological fluids, particularly blood. When referring to a cyclic peptide of the invention having a modified disulfide bond, it will be understood in view of the disclosure herein that the modification can be to the disulfide bond directly, such that one or both sulfur atoms are replaced and/or modified, or can be a modification at one or more atoms adjacent to, or in the vicinity of, the sulfur atoms of the disulfide bond, provided such modification provides improved stability according to the invention.

The size of a cyclic peptide ring generally ranges from 5 to about 15 residues, preferably from 5 to 10 residues. Additional residue(s) may be present on the N-terminal and/or C-terminal side of a CAR sequence, and may be derived from sequences that flank the native CAR sequence, with or without amino acid substitutions and/or other modifications. Alternatively, additional residues present on one or both sides of the CAR sequence(s) may be unrelated to an endogenous sequence (e.g., residues that facilitate cyclization, purification or other manipulation and/or residues having a targeting or other function).

In one illustrative class of modifications that improves the stability of disulfide-containing cyclic peptides, a modified disulfide bond comprises one or more steric R groups on a carbon atom adjacent to one or both sulfur atoms of the disulfide bond, as illustratively set forth in the structure below:

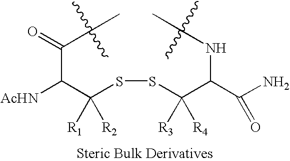

wherein R₁, R₂, R₃ and R₄ are the same or different and independently selected from H, alkyl, alkenyl, alkynyl, cycloalkyl, cycloalkyl-alkyl, heterocyclyl, heterocyclyl-alkyl, aryl, alkylaryl, arylalkyl, heteroaryl, alkylheteroaryl, heteroarylalkyl, alkoxy, alkylalkoxy, alkylthio, alkylamino, carbocycle, substituted carbocycle, heterocycle, substituted heterocycle, or —(CH₂)ₙ—X—(CH₂)ₘ— where n and m are independently 1, 2, 3 or 4 and X═O, S, NH, or N-alkyl. In a particular embodiment, R₁, R₂, R₃ and R₄ are H or C₁-C₄ alkyl. In another embodiment, R₁ and R₂ are H and R₃ and R₄ are C₁-C₄ alkyl. In another embodiment, R₁ and R₂ are C₁-C₄ alkyl and R₃ and R₄ are H.

In another illustrative class of modified cyclic peptides of the invention, the disulfide bond of a cyclic peptide is modified by dithioalkylation to form aromatic or alcohol derivatives as illustratively set forth in the structures below:

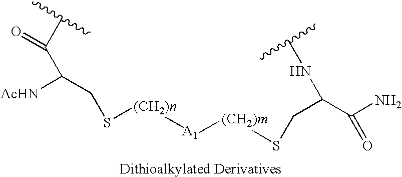

wherein n and m are independently 1, 2, 3 or 4 and A₁ is aryl, substituted aryl, heteraryl, substituted heteroaryl, carbonyl, a carbon bearing an alcohol, a carbon bearing an alkylether, a carbon bearing an arylether, a carbon bearing an heteroarylether, a carbon bearing an alkylester, a carbon bearing an arylester, a carbon bearing an heteroarylester, a carbon bearing an amine, a carbon bearing an alkylamine, a carbon bearing an arylamine, a carbon bearing a heteroarylamine, a carbon bearing an amide, a carbon bearing an alkylamide.

In another class of modifications, the disulfide bond is modified to form a thioether derivative, as illustratively set forth in the structures below:

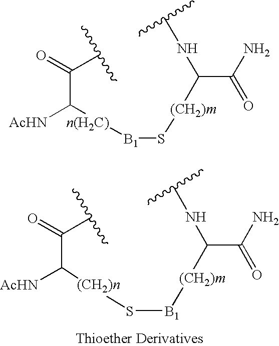

wherein n and m are independently 1, 2, 3 or 4; and B₂ is aryl or heteroaryl.

In yet another class of disulfide modifications, both sulfur atoms of the disulfide linkage are replaced to produce an amide derivative, as illustrated in the structure below:

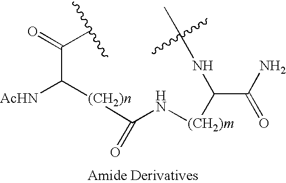

wherein n and m are independently 1, 2, 3 or 4. In a particular embodiment, n is 2 and m is 1.

In another embodiment of the invention, cyclic peptides as described herein are modified with polyethylene glycol. In one such embodiment, a pegylated cyclic peptide of the invention has a structure as set forth below:

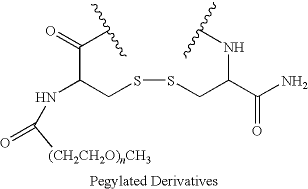

wherein n is in a range from about 1 to 100, 1 to 50 or 1 to 25. In certain embodiments, the pegylated cyclic peptide incorporates PEG molecules having molecular weights in the range of 50 to 50,000, 50 to 10,000 or 50 to 1000. In a more particular embodiment, the cyclic peptide incorporates PEG molecules having molecular weights in the range of 100-500. It is intended that pegylation, e.g., at the N-terminal cysteine of a cyclic peptide of the invention, may be employed either alone or in combination with the other modifications described herein.

According to a more particular embodiment of the invention, the modified disulfide bond of a cyclic peptide of the invention is selected from a structure as set forth below:

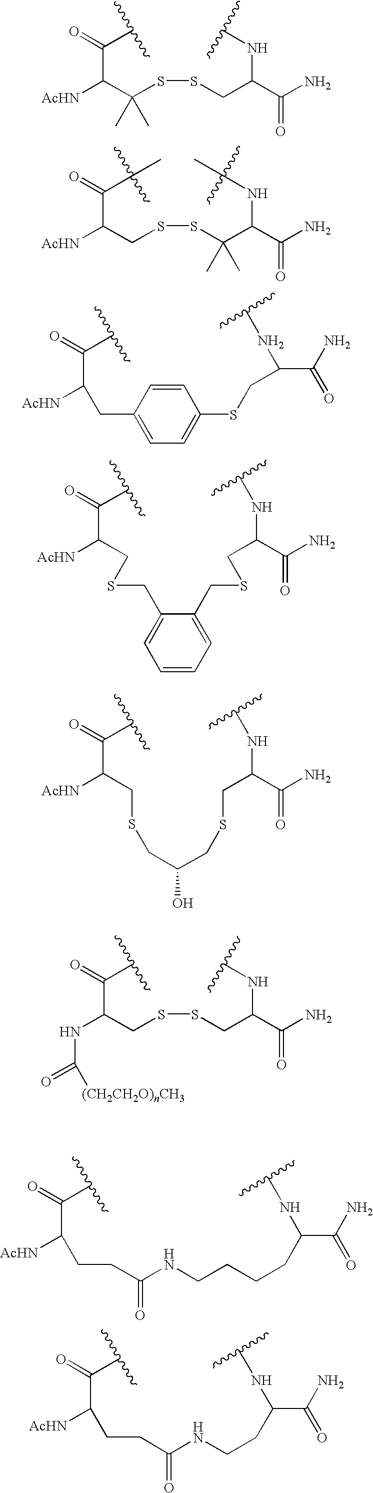

Unless otherwise stated the following terms used in the specification and claims have the meanings set forth below. Further, in the structures presented above and elsewhere herein, although the N-terminal cysteines of the cyclic peptides are shown as acetylated and the C-terminal cysteines are shown as amidated, these groups are optional and may but need not be present in the modified cyclic peptides of the invention.

“Alkyl” refers to a saturated straight or branched hydrocarbon radical of one to six carbon atoms, preferably one to four carbon atoms, e.g., methyl, ethyl, propyl, 2-propyl, n-butyl, iso-butyl, tert-butyl, pentyl, hexyl, and the like, preferably methyl, ethyl, propyl, or 2-propyl. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclohexyl, and the like; while unsaturated cyclic alkyls include cyclopentenyl, cyclohexenyl, —CH₂-cyclohexenyl, and the like. Cyclic alkyls are also referred to herein as a “cycloalkyl.” Unsaturated alkyls contain at least one double or triple bond between adjacent carbon atoms (referred to as an “alkenyl” or “alkynyl”, respectively.) Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like, preferably methylene, ethylene, or propylene.

“Cycloalkyl” refers to a saturated cyclic hydrocarbon radical of three to eight carbon atoms, e.g., cyclopropyl, cyclobutyl, cyclopentyl or cyclohexyl.

“Alkoxy” means a radical —ORₐ where Rₐ is an alkyl as defined above, e.g., methoxy, ethoxy, propoxy, butoxy and the like.

“Halo” means fluoro, chloro, bromo, or iodo.

“Haloalkyl” means alkyl substituted with one or more, preferably one, two or three, same or different halo atoms, e.g., —CH₂Cl, —CF₃, —CH₂CF₃, —CH₂CCl₃, and the like.

“Haloalkoxy” means a radical —OR_{b} where R_{b} is an haloalkyl as defined above, e.g., trifluoromethoxy, trichloroethoxy, 2,2-dichloropropoxy, and the like.

“Acyl” means a radical —C(O)R_{c} where R_{c} is hydrogen, alkyl, or haloalkyl as defined herein, e.g., formyl, acetyl, trifluoroacetyl, butanoyl, and the like.

“Aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The aryl group may be substituted or unsubstituted. When substituted, the aryl group is substituted with one or more, more preferably one, two or three, even more preferably one or two substituents independently selected from the group consisting of alkyl, haloalkyl, halo, hydroxy, alkoxy, mercapto, alkylthio, cyano, acyl, nitro, phenoxy, heteroaryl, heteroaryloxy, haloalkyl, haloalkoxy, carboxy, alkoxycarbonyl, amino, alkylamino or dialkylamino.

“Heteroaryl” refers to a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, and carbazole. The heteroaryl group may be substituted or unsubstituted. When substituted, the heteroaryl group is substituted with one or more, more preferably one, two or three, even more preferably one or two substituents independently selected from the group consisting of alkyl, haloalkyl, halo, hydroxy, alkoxy, mercapto, alkylthio, cyano, acyl, nitro, haloalkyl, haloalkoxy, carboxy, alkoxycarbonyl, amino, alkylamino or dialkylamino.

“Carbocycle” refers to a saturated, unsaturated or aromatic ring system having 3 to 14 ring carbon atoms. The term “carbocycle”, whether saturated or partially unsaturated, also refers to rings that are optionally substituted. The term “carbocycle” includes aryl. The term “carbocycle” also includes aliphatic rings that are fused to one or more aromatic or nonaromatic rings, such as in a decahydronaphthyl or tetrahydronaphthyl, where the radical or point of attachment is on the aliphatic ring. The carbocycle group may be substituted or unsubstituted. When substituted, the carbocycle group is substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Heterocycle” refers to a saturated, unsaturated or aromatic cyclic ring system having 3 to 14 ring atoms in which one, two or three ring atoms are heteroatoms selected from N, O, or S(O)ₘ (where m is an integer from 0 to 2), the remaining ring atoms being C, where one or two C atoms may optionally be replaced by a carbonyl group. The term “heterocycle” includes heteroaryl. The heterocyclyl ring may be optionally substituted independently with one or more, preferably one, two, or three substituents selected from alkyl (wherein the alkyl may be optionally substituted with one or two substituents), haloalkyl, cycloalkylamino, cycloalkylalkyl, cycloalkylaminoalkyl, cycloalkylalkylaminoalkyl, cyanoalkyl, halo, nitro, cyano, hydroxy, alkoxy, amino, alkylamino, dialkylamino, hydroxyalkyl, carboxyalkyl, aminoalkyl, alkylaminoalkyl, dialkylaminoalkyl, aralkyl, heteroaralkyl, aryl, heteroaryl, carbocycle, heterocycle (wherein the aryl, heteroaryl, carbocycle or heterocycle may be optionally substituted), aralkyl, heteroaralkyl, saturated or unsaturated heterocycloamino, saturated or unsaturated heterocycloaminoalkyl, and —COR_{d} (where R_{d} is alkyl). More specifically the term heterocyclyl includes, but is not limited to, tetrahydropyranyl, 2,2-dimethyl-1,3-dioxolane, piperidino, N-methylpiperidin-3-yl, piperazino, N-methylpyrrolidin-3-yl, pyrrolidino, morpholino, 4-cyclopropylmethylpiperazino, thiomorpholino, thiomorpholino-1-oxide, thiomorpholino-1,1-dioxide, 4-ethyloxycarbonylpiperazino, 3-oxopiperazino, 2-imidazolidone, 2-pyrrolidinone, 2-oxohomopiperazino, tetrahydropyrimidin-2-one, and the derivatives thereof. In certain embodiments, the heterocycle group is optionally substituted with one or two substituents independently selected from halo, alkyl, alkyl substituted with carboxy, ester, hydroxy, alkylamino, saturated or unsaturated heterocycloamino, saturated or unsaturated heterocycloaminoalkyl, or dialkylamino.

“Optional” or “optionally” means that the subsequently described event or circumstance may but need not occur, and that the description includes instances where the event or circumstance occurs and instances in which it does not. For example, “heterocyclic group optionally substituted with an alkyl group” means that the alkyl may but need not be present, and the description includes situations where the heterocycle group is substituted with an alkyl group and situations where the heterocyclo group is not substituted with the alkyl group.

Lastly, the term “substituted” as used herein means any of the above groups (e.g., alkyl, alkylene, cycloalkyl, alkoxy, haloalkyl, haloalkoxy, acyl, aryl, heteroaryl, carbocycle, heterocycle, etc.) wherein at least one hydrogen atom is replaced with a substituent. In the case of an oxo substituent (“═O”) two hydrogen atoms are replaced. “Substituents” within the context of this invention include halogen, hydroxy, oxo, cyano, nitro, amino, alkylamino, dialkylamino, alkyl, alkoxy, thioalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₑR_{f}, —NRₑC(═O)R_{f}, —NRₑC(═O)NRₑR_{f}, —NRₑC(═O)OR_{f}—NRₑSO₂R_{f}, —ORₑ, —C(═O)Rₑ—C(═O)ORₑ, —C(═O)NRₑR_{f}, —OC(═O)NRₑR_{f}, —SH, —SRₑ, —SORₑ, —S(═O)₂Rₑ, —OS(═O)₂Rₑ, —S(═O)₂ORₑ, wherein Rₑ and R_{f} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

### Cell Adhesion Recognition (CAR) Sequences and Cyclic Peptides Containing Same

Cell adhesion recognition (CAR) sequences, and their use in disulfide-containing cyclic peptides, have been extensively described and characterized. Any such compounds known in the art may be modified according to the present invention. Particularly illustrative CAR sequences, as well as cyclic peptides containing same which can be modified according to the present invention, are illustratively described below. Each of the references cited, and the compounds therein described, are incorporated herein by reference in their entireties.

a. HAV CAR Sequences

CAR sequences comprising the tripeptide sequence, HAV, as well as HAV-containing cyclic peptides, have been extensively described, e.g., U.S. Pat. Nos. 6,031,072; 6,417,325; 6,465,427; 6,780,845; 6,203,788; 6,203,788; and WO05/012348, the contents of which are incorporated herein by reference in their entireties. Also describe therein are analogues, derivatives and peptidomimetics of HAV CAR sequences and cyclic peptides thereof, as well as antibodies that bind such molecules.

Such cyclic peptides generally comprise at least the cadherin CAR sequence, HAV, and are cyclized via a disulfide linkage. Particular HAV-containing cyclic peptides which may be modified according to the present invention have the following formula:

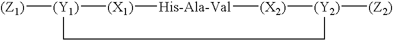

wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; and wherein a covalent disulfide bond is formed between residues Y₁ and Y₂ which may be modified as described herein; and wherein Z₁ and Z₂ are optional, and if present, are independently selected from the group consisting of amino acid residues and combinations thereof in which the residues are linked by peptide bonds.

Within certain embodiments, a cyclic peptide may optionally comprise an N-acetyl group (i.e., the amino group present on the amino terminal residue of the peptide prior to cyclization is acetylated) or an N-formyl group (i.e., the amino group present on the amino terminal residue of the peptide prior to cyclization is formylated), or the amino group present on the amino terminal residue of the peptide prior to cyclization is mesylated. One illustrative cyclic peptide, for example, is N-Ac-CHAVC-NH₂ (SEQ ID NO: 1). Another illustrative cyclic peptide is N-Ac-CHAVC-Y-NH₂ (SEQ ID NO:2). Other illustrative cyclic peptides which may be modified according to the present invention include, but are not limited to: N-Ac-CHAVDC-NH₂ (SEQ ID NO:3), N-Ac-CHAVDIC-NH₂ (SEQ ID NO:4), N-Ac-CHAVDINC-NH₂ (SEQ ID NO:5), N-Ac-CHAVDINGC-NH₂ (SEQ ID NO:6), N-Ac-CAHAVC-NH₂ (SEQ ID NO:7), N-Ac-CAHAVDC-NH₂ (SEQ ID NO:8), N-Ac-CAHAVDIC-NH₂ (SEQ ID NO:9), N-Ac-CRAHAVDC-NH₂ (SEQ ID NO:10), N-Ac-CLRAHAVC-NH₂ (SEQ ID NO:11), N-Ac-CLRAHAVDC-NH₂ (SEQ ID NO: 12), N-Ac-CSHAVC-NH₂ (SEQ ID NO: 13), N-Ac-CFSHAVC-NH₂ (SEQ ID NO: 14), N-Ac-CLFSHAVC-NH₂ (SEQ ID NO: 15), N-Ac-CHAVSC-NH₂ (SEQ ID NO: 16), N-Ac-CSHAVSC-NH₂ (SEQ ID NO:17), N-Ac-CSHAVSSC-NH₂ (SEQ ID NO:18), N-Ac-CHAVSSC-NH₂ (SEQ ID NO:19), N-Ac-KHAVD-NH₂ (SEQ ID NO:20), N-Ac-DHAVK-NH₂ (SEQ ID NO:21), N-Ac-KHAVE-NH₂ (SEQ ID NO:22), N-Ac-AHAVDI-NH₂ (SEQ ID NO:23), N-Ac-SHAVDSS-NH₂ (SEQ ID NO:24), N-Ac-KSHAVSSD-NH₂ (SEQ ID NO:25), N-Ac-CHAVC-S-NH₂ (SEQ ID NO:26), N-Ac-S-CHAVC-NH₂ (SEQ ID NO:27), N-Ac-CHAVC-SS-NH₂ (SEQ ID NO:28), N-Ac-S-CHAVC-S-NH₂ (SEQ ID NO:29), N-Ac-CHAVC-T-NH₂ (SEQ ID NO:30), N-Ac-CHAVC-E-NH₂ (SEQ ID NO:31), N-Ac-CHAVC-D-NH₂ (SEQ ID NO:32), N-Ac-CHAVYC-NH₂ (SEQ ID NO:33), CH₃-SO₂-HN-CHAVC-Y-NH₂ (SEQ ID NO:34), CH₃-SO₂-HN-CHAVC-NH₂ (SEQ ID NO:35), HC(O)—NH-CHAVC-NH₂ (SEQ ID NO:36), N-Ac-CHAVPen-NH₂ (SEQ ID NO:37), N-Ac-PenHAVC-NH₂ (SEQ ID NO:38) and N-Ac-CHAVPC-NH₂ (SEQ ID NO:39).

b. Trp-containing CAR Sequences

Additional CAR sequences useful in the present invention include Trp-containing CAR sequences that modulate classical cadherins, as well as peptidomimetics, analogues and derivatives thereof, such as those described in U.S. patent application Ser. No. 10/714,556; US Patent Publication No. 2005/0129676, and PCT Publication No. WO04/044000, the contents of which are incorporated herein by reference in their entireties.

For example, illustrative Trp-containing CAR sequences may comprise the consensus sequence: Asp/Glu-Trp-Val-Ile/Val/Met-Pro/Ala-Pro (SEQ ID NO:40), wherein “Asp/Glu” is an amino acid that is either Asp or Glu, “Ile/Val/Met” is an amino acid that is Ile, Val or Met, and “Pro/Ala” is either Pro or Ala. Particular Trp-containing CAR sequences or conservative analogues thereof include, but are not limited to, DWV, DWVI (SEQ ID NO:41), DWVV (SEQ ID NO: 42), DWVM (SEQ ID NO:43), DWVIP (SEQ ID NO:44), DWVIA (SEQ ID NO:45), DWVVP (SEQ ID NO:46), DWVVPP (SEQ ID NO:47), DWVVAP (SEQ ID NO:48), DWVMPP (SEQ ID NO:49), DWVMAP (SEQ ID NO:50), EWV, EWVI (SEQ ID NO:51), EWVV (SEQ ID NO:52), EWVM (SEQ ID NO:53), EWVIP (SEQ ID NO:54), EWVIA (SEQ ID NO:55), EWVVP (SEQ ID NO:56), EWVVPP (SEQ ID NO:57), EWVVAP (SEQ ID NO:58), EWVMPP (SEQ ID NO:59), EWVMAP (SEQ ID NO:60), WVI, WVIP (SEQ ID NO:61), WVIA (SEQ ID NO:62), WVV, WVVP (SEQ ID NO:63), WVVA (SEQ ID NO:64), WVM, WVMP (SEQ ID NO:65), WVMA (SEQ ID NO:66), WVIPP (SEQ ID NO:67), WVIAP (SEQ ID NO:68), WVVPP (SEQ ID NO:69), WVVAP (SEQ ID NO:70), WVMPP (SEQ ID NO:71), WVMAP (SEQ ID NO:72), DWI, DWII (SEQ ID NO:73), DWIV (SEQ ID NO:74), DWIM (SEQ ID NO:75), DWIIP (SEQ ID NO:76), DWIIA (SEQ ID NO:77), DWIVP (SEQ ID NO:78), DWIVPP (SEQ ID NO:79), DWIVAP (SEQ ID NO:80), DWIMPP (SEQ ID NO:81), DWIMAP (SEQ ID NO:82), EWI, EWII (SEQ ID NO:83), EWIV (SEQ ID NO:84), EWIM (SEQ ID NO:85), EWIIP (SEQ ID NO:86), EWIIA (SEQ ID NO:87), EWIVP (SEQ ID NO:88), EWIVPP (SEQ ID NO:89), EWIVAP (SEQ ID NO:90), EWIMPP (SEQ ID NO:91), EWIMAP (SEQ ID NO:92), WII, WIIP (SEQ ID NO:93), WIIA (SEQ ID NO:94), WIV, WIVP (SEQ ID NO:95), WIVA (SEQ ID NO:96), WIM, WIMP (SEQ ID NO:97), WIMA (SEQ ID NO:98), WIIPP (SEQ ID NO:99), WIIAP (SEQ ID NO:100), WIVPP (SEQ ID NO:101), WIVAP (SEQ ID NO:102), WIMPP (SEQ ID NO:103), WIMAP (SEQ ID NO:104), DWL, DWLI (SEQ ID NO:105), DWLV (SEQ ID NO:106), DWLM (SEQ ID NO:107), DWLIP (SEQ ID NO:108), DWLIA (SEQ ID NO:109), DWLVP (SEQ ID NO:1101), DWLVPP (SEQ ID NO:111), DWLVAP (SEQ ID NO:112), DWLMPP (SEQ ID NO:113), DWLMAP (SEQ ID NO:114), EWL, EWLI (SEQ ID NO:115), EWLV (SEQ ID NO:116), EWLM (SEQ ID NO:117), EWLIP (SEQ ID NO:118), EWLIA (SEQ ID NO:119), EWLVP (SEQ ID NO:120), EWLVPP (SEQ ID NO:121), EWLVAP (SEQ ID NO:122), EWLMPP (SEQ ID NO:123), EWLMAP (SEQ ID NO:124), WLI, WLIP (SEQ ID NO:125), WLIA (SEQ ID NO:126), WLV, WLVP (SEQ ID NO:127), WLVA (SEQ ID NO:128), WLM, WLMP (SEQ ID NO:129), WLMA (SEQ ID NO:130), WLIPP (SEQ ID NO:131), WLIAP (SEQ ID NO:132), WLVPP (SEQ ID NO:133), WLVAP (SEQ ID NO:134), WLMPP (SEQ ID NO:135), WLMAP (SEQ ID NO:136), DWVL (SEQ ID NO:137), DWIL (SEQ ID NO:138), DWLL (SEQ ID NO:139), EWVL (SEQ ID NO:140), EWIL (SEQ ID NO:141), EWLL (SEQ ID NO:142), DWVLP (SEQ ID NO:143), DWILP (SEQ ID NO:144), DWLLP (SEQ ID NO:145), EWVLP (SEQ ID NO:146), EWILP (SEQ ID NO:147), EWLLP (SEQ ID NO:148), DWVLA (SEQ ID NO:149), DWILA (SEQ ID NO:150), DWLLA (SEQ ID NO:151), EWVLA (SEQ ID NO:152), EWILA (SEQ ID NO:153), EWLLA (SEQ ID NO:154), DWVLPP (SEQ ID NO:155), DWILPP (SEQ ID NO:156), DWLLPP (SEQ ID NO:157), EWVLPP (SEQ ID NO:158), EWILPP (SEQ ID NO:159), EWLLPP (SEQ ID NO:160), DWVLAP (SEQ ID NO:161), DWILAP (SEQ ID NO:162), DWLLAP (SEQ ID NO:163), EWVLAP (SEQ ID NO:164), EWILAP (SEQ ID NO:165), EWLLAP (SEQ ID NO:166), WVL, WIL, WLL, WVLP (SEQ ID NO:167), WILP (SEQ ID NO:168), WLLP (SEQ ID NO:169), WVLA (SEQ ID NO:170), WILA (SEQ ID NO:171), WLLA (SEQ ID NO:172), WVLPP (SEQ ID NO:173), WILPP (SEQ ID NO:174), WLLPP (SEQ ID NO:175), WVLAP (SEQ ID NO:176), WILAP (SEQ ID NO:177), and WLLAP (SEQ ID NO:178).

Illustrative examples of Trp-containing CAR sequences that are present in cyclic peptide structures and which may be modified according to the present invention include, but are not limited to, the following structures:

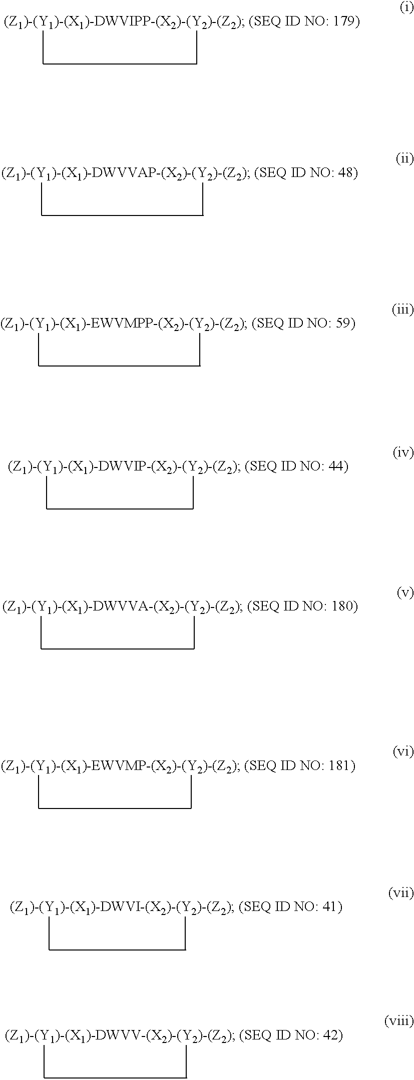

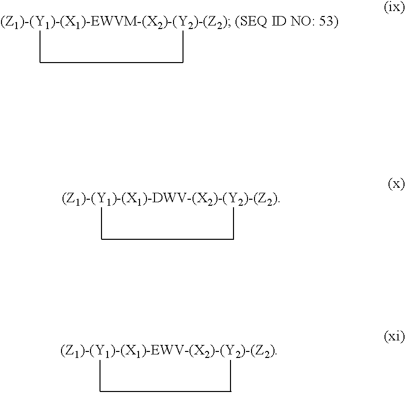

In these structures, X₁ and X₂ are optional, and if present, are amino acid residues or combinations of amino acid residues linked by peptide bonds. X₁ and X₂ may be identical to, or different from, each other. In general, X₁ and X₂ independently range in size from 0 to 10 residues, such that the sum of residues contained within X₁ and X₂ ranges from 1 to 12. A covalent disulfide bond is formed between residues Y₁ and Y₂ which may be modified as described herein in order to improve stability of the compound. Z₁ and Z₂ are optional, and if present, are amino acid residues or combinations of amino acid residues linked by peptide bonds. Z₁ and Z₂ may be identical to, or different from, each other.

Other Trp-containing CAR sequences include those that modulate non-classical and atypical cadherins, as well as peptidomimetics, analogues and derivatives thereof, such as those described in US Patent Publication No. 2004/0175361, the content of which is incorporated herein by reference in its entirety.

For example, certain atypical cadherin Trp-containing CAR sequences share the consensus sequence:

(SEQ ID NO:268)

Gly/Asp/Ser-Trp-Val/Ile/Met-Trp-Asn-Gln

Within the consensus sequence, “Gly/Asp/Ser” indicates an amino acid that is Gly, Asp or Ser; and “Val/Ile/Met” indicates an amino acid that is Val, Ile or Met. Trp-containing CAR sequences further include portions of such representative Trp-containing CAR sequences, as well as polypeptides that comprise at least a portion of such sequences. Additional atypical cadherin Trp-containing CAR sequences may be identified based on sequence homology to the atypical cadherin Trp-containing CAR sequences provided herein, and based on the ability of a peptide comprising such a sequence to modulate an atypical cadherin-mediated function within a representative assay described herein. Within certain embodiments, the CAR sequence comprises at least three, four, five and six consecutive residues of an atypical cadherin Trp-containing CAR sequence that satisfies the above consensus sequence.

Exemplary Trp-containing CAR sequences for atypical cadherins include, but are not limited to GWV, GWVW (SEQ ID NO:269), GWVWN (SEQ ID NO:270), GWVWNQ (SEQ ID NO:271), WVW, WVWN (SEQ ID NO:272), WVWNQ (SEQ ID NO:273), DWI, DWIW (SEQ ID NO:274), DWIWN (SEQ ID NO:275), DWIWNQ (SEQ ID NO:276), WIW, WIWN (SEQ ID NO:277), WIWNQ (SEQ ID NO:278), SWM, SWMW (SEQ ID NO:279), SWMWN (SEQ ID NO:280), SWMWNQ (SEQ ID NO:281), WMW, WMWN (SEQ ID NO:282), WMWNQ (SEQ ID NO:283), SWV, SWVW (SEQ ID NO:284), SWVWN (SEQ ID NO:285), SWVWNQ (SEQ ID NO:286), GWM, GWMW (SEQ ID NO:287), GWMWN (SEQ ID NO:288), GWMWNQ (SEQ ID NO:289), AWV, AWVI (SEQ ID NO:290), AWVIP (SEQ ID NO:291), AWVIPP (SEQ ID NO:292), WVI, WVIP (SEQ ID NO:293), WVIPP (SEQ ID NO:294), GWVWNQF (SEQ ID NO:295), GWVWNQFF (SEQ ID NO:296), GWVWNQFFV (SEQ ID NO:297), WVWNQF (SEQ ID NO:298), WVWNQFF (SEQ ID NO:299), WVWNQFFV (SEQ ID NO:300), RGW, RGWV (SEQ ID NO:301), RGWVW (SEQ ID NO:302), RGWVWN (SEQ ID NO:303), RGWVWNQ (SEQ ID NO:304), RGWVWNQF (SEQ ID NO:305), RGWVWNQFF (SEQ ID NO:306), RGWVWNQFFV (SEQ ID NO:307), KRGW (SEQ ID NO:308), KRGWV (SEQ ID NO:309), KRGWVW (SEQ ID NO:310), KRGWVWN (SEQ ID NO:311), KRGWVWNQ (SEQ ID NO:312), KRGWVWNQF (SEQ ID NO:313), KRGWVWNQFF (SEQ ID NO:314), KRGWVWNQFFV (SEQ ID NO:315), DWIWNQM (SEQ ID NO:316), DWIWNQMH (SEQ ID NO:317), DWIWNQMHI (SEQ ID NO:318), WIWNQM (SEQ ID NO:319), WIWNQMH (SEQ ID NO:320), WIWNQMHI (SEQ ID NO:321), RDW, RDWI (SEQ ID NO:322), RDWIW (SEQ ID NO:323), RDWIWN (SEQ ID NO:324), RDWIWNQ (SEQ ID NO:325), RDWIWNQM (SEQ ID NO:326), RDWIWNQMH (SEQ ID NO:327), RDWIWNQMHI (SEQ ID NO:328), KRDW (SEQ ID NO:329), KRDWI (SEQ ID NO:330), KRDWIW (SEQ ID NO:331), KRDWIWN (SEQ ID NO:332), KRDWIWNQ (SEQ ID NO:333), KRDWIWNQM (SEQ ID NO:334), KRDWIWNQMH (SEQ ID NO:335), KRDWIWNQMHI (SEQ ID NO:336), SWMWNQF (SEQ ID NO:337), SWMWNQFF (SEQ ID NO:338), SWMWNQFFL (SEQ ID NO:339), WMWNQF (SEQ ID NO:340), WMWNQFF (SEQ ID NO:341), WMWNQFFL (SEQ ID NO:342), RSW, RSWM (SEQ ID NO:343), RSWMW (SEQ ID NO:344), RSWMWN (SEQ ID NO:345), RSWMWNQ (SEQ ID NO:346), RSWMWNQF (SEQ ID NO:347), RSWMWNQFF (SEQ ID NO:348), RSWMWNQFFL (SEQ ID NO:349), KRSW (SEQ ID NO:350), KRSWM (SEQ ID NO:351), KRSWMW (SEQ ID NO:352), KRSWMWN (SEQ ID NO:353), KRSWMWNQ (SEQ ID NO:354), KRSWMWNQF (SEQ ID NO:355), KRSWMWNQFF (SEQ ID NO:356), KRSWMWNQFFL (SEQ ID NO:357), SWVWNQF (SEQ ID NO:358), SWVWNQFF (SEQ ID NO:359), SWVWNQFFV (SEQ ID NO:360), WVWNQF (SEQ ID NO:361), WVWNQFF (SEQ ID NO:362), WVWNQFFV (SEQ ID NO:363), RSWV (SEQ ID NO:364), RSWVW (SEQ ID NO:365), RSWVWN (SEQ ID NO:366), RSWVWNQ (SEQ ID NO:367), RSWVWNQF (SEQ ID NO:368), RSWVWNQFF (SEQ ID NO:369), RSWVWNQFFV (SEQ ID NO:370), KRSWV (SEQ ID NO:371), KRSWVW (SEQ ID NO:372), KRSWVWN (SEQ ID NO:373), KRSWVWNQ (SEQ ID NO:374), KRSWVWNQF (SEQ ID NO:375), KRSWVWNQFF (SEQ ID NO:376), KRSWVWNQFFV (SEQ ID NO:377), GWVWNQM (SEQ ID NO:378), GWVWNQMF (SEQ ID NO:379), GWVWNQMFV (SEQ ID NO:380), RGWVWNQM (SEQ ID NO:381), RGWVWNQMF (SEQ ID NO:382), RGWVWNQMFV (SEQ ID NO:383), KRGWVWNQM (SEQ ID NO:384), KRGWVWNQMFV (SEQ ID NO:385), GWVWNQFFL (SEQ ID NO:386), RGWVWNQFFL (SEQ ID NO:387), KRGWVWNQFFL (SEQ ID NO:388), AWVIPPI (SEQ ID NO:389), AWVIPPIS (SEQ ID NO:390), AWVIPPISV (SEQ ID NO:391), WVIPPI (SEQ ID NO:392), WVIPPIS (SEQ ID NO:393), WVIPPISV (SEQ ID NO:394), RAW, RAWV (SEQ ID NO:395), RAWVI (SEQ ID NO:396), RAWVIP (SEQ ID NO:397), RAWVIPP (SEQ ID NO:398), RAWVIPPI (SEQ ID NO:399), RAWVIPPIS (SEQ ID NO:400), RAWVIPPISV (SEQ ID NO:401), KRAW (SEQ ID NO:402), KRAWV (SEQ ID NO:403), KRAWVI (SEQ ID NO:404), KRAWVIP (SEQ ID NO:405), KRAWVIPP (SEQ ID NO:406), KRAWVIPPI (SEQ ID NO:407), KRAWVIPPIS (SEQ ID NO:408), VWN, VWNQ (SEQ ID NO:409), VWNQM (SEQ ID NO:410), VWNQF (SEQ ID NO:411), VWNQMF (SEQ ID NO:412), VWNQFF (SEQ ID NO:413), WNQ, WNQM (SEQ ID NO:414), WNQF (SEQ ID NO:415), WNQFF (SEQ ID NO:416), IWN, IWNQ (SEQ ID NO:417), IWNQM (SEQ ID NO:418), IWNQMH (SEQ ID NO:419), WNQM (SEQ ID NO:420), WNQMH (SEQ ID NO:421), MWN, MWNQ (SEQ ID NO:422), MWNQF (SEQ ID NO:423), and MWNQFF (SEQ ID NO:424).

In addition, certain illustrative atypical cadherin CAR sequences that are present within a cyclic peptide ring comprise the sequence G/S/D-W-V/M/I-W-N-Q (SEQ ID NO:268), the sequence AWVIPP (SEQ ID NO:292), or a portion thereof. Particular cyclic peptides of this class that can be modified according to the present invention include, but are not limited to, those having the following formula:

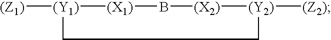

In this formula, B represents an amino acid sequence selected from the following CAR sequences: DWIWNQ (SEQ ID NO:276), SWMWNQ (SEQ ID NO:281), SWVWNQ (SEQ ID NO:286), GWVWNQ (SEQ ID NO:271), AWVIPP (SEQ ID NO:292), GWVWN (SEQ ID NO:270), DWIWN (SEQ ID NO:275), SWMWN (SEQ ID NO:280), SWVWN (SEQ ID NO:285), GWVWN (SEQ ID NO:270), AWVIP (SEQ ID NO:291), GWVW (SEQ ID NO:269), DWIW (SEQ ID NO:274), SWMW (SEQ ID NO:279), SWVW (SEQ ID NO:284), GWVW (SEQ ID NO:269), AWVI (SEQ ID NO:290), GWV, DWI, SWM, SWV, GWV, AWV, VWN, VWNQ (SEQ ID NO:409), VWNQM (SEQ ID NO:410), VWNQF (SEQ ID NO:411), VWNQMF (SEQ ID NO:412), VWNQFF (SEQ ID NO:413), WNQ, WNQM (SEQ ID NO:414), WNQF (SEQ ID NO:415), WNQFF (SEQ ID NO:416), IWN, IWNQ (SEQ ID NO:417), IWNQM (SEQ ID NO:418), IWNQMH (SEQ ID NO:419), WNQM (SEQ ID NO:420), WNQMH (SEQ ID NO:421), MWN, MWNQ (SEQ ID NO:422), MWNQF (SEQ ID NO:423), and MWNQFF (SEQ ID NO:424). X₁ and X₂ are optional, and if present, are amino acid residues or combinations of amino acid residues linked by peptide bonds. X₁ and X₂ may be identical to, or different from, each other. In general, X₁ and X₂ independently range in size from 0 to 10 residues, such that the sum of residues contained within X₁ and X₂ ranges from 1 to 12. A disulfide covalent bond is formed between residues Y₁ and Y₂, which may be modified as described herein. Z₁ and Z₂ are optional, and if present, are amino acid residues or combinations of amino acid residues linked by peptide bonds. Z₁ and Z₂ may be identical to, or different from, each other.

c. HAV-BM CAR sequences

Other CAR sequences useful according to the present invention include sequences referred to as HAV-binding motif (HAV-BM) sequences, such as those described, e.g., in U.S. Pat. Nos. 6,277,824; 6,472,368; and 6,806,255. Such agents generally comprise an HAV-BM sequence, or an analogue, peptidomimetic or derivative thereof. In a particular embodiment, the HAV-BM sequence comprises the sequence: (a) Ile/Val-Phe-Aaa-Ile-Baa-Caa-Daa-Ser/Thr-Gly-Eaa-Leu/Met (SEQ ID NO:182), wherein Aaa, Baa, Caa, Daa and Eaa are independently selected from the group consisting of amino acid residues; or comprises the sequence Trp-Leu-Aaa-Ile-Asp/Asn-Baa-Caa-Daa-Gly-Gln-Ile (SEQ ID NO:183), wherein Aaa, Baa, Caa and Daa are independently selected from the group consisting of amino acid residues.

Certain illustrative HAV-BM sequences include, but are not limited to, sequences selected from the group consisting of: IFIINPISGQL (SEQ ID NO: 184), IFILNPISGQL (SEQ ID NO:185), VFAVEKETGWL (SEQ ID NO: 186), VFSINSMSGRM (SEQ ID NO:187), VFIIERETGWL (SEQ ID NO:188), VFTIEKESGWL (SEQ ID NO: 189), VFNIDSMSGRM (SEQ ID NO: 190), WLKIDSVNGQI (SEQ ID NO: 191), WLKIDPVNGQI (SEQ ID NO: 192), WLAMDPDSGQV (SEQ ID NO:193), WLHINATNGQI (SEQ ID NO: 194), WLEINPDTGAI (SEQ ID NO: 195), WLAVDPDSGQI (SEQ ID NO: 196), WLEINPETGAI (SEQ ID NO: 197), WLHINTSNGQI (SEQ ID NO: 198), NLKIDPVNGQI (SEQ ID NO:199), LKIDPVNGQI (SEQ ID NO:200) and analogues of the foregoing sequences that retain at least seven consecutive residues (e.g., INPISGQ (SEQ ID NO:201), LNPISGQ (SEQ ID NO:202), IDPVSGQ (SEQ ID NO:203) or KIDPVNGQ (SEQ ID NO:204)), wherein the ability of the analogue to modulate a cadherin-mediated process is not diminished. Alternatively, an agent may be an HAV-BM sequence that comprises at least five consecutive residues of a peptide selected from the group consisting of INPISGQ (SEQ ID NO:201), LNPISGQ (SEQ ID NO:202), NLKIDPVNGQI (SEQ ID NO:203) and WLKIDPVNGQI (SEQ ID NO:204). For example, the agent may comprise a sequence selected from the group consisting of PISGQ (SEQ ID NO:205), PVNGQ (SEQ ID NO:206), PVSGR (SEQ ID NO:207), IDPVN (SEQ ID NO:208), INPIS (SEQ ID NO:209) and KIDPV (SEQ ID NO:210).

Particular cyclic peptides which contain HAV-BM CAR sequences and which may be modified as described herein include, but are not limited to, the following structures:

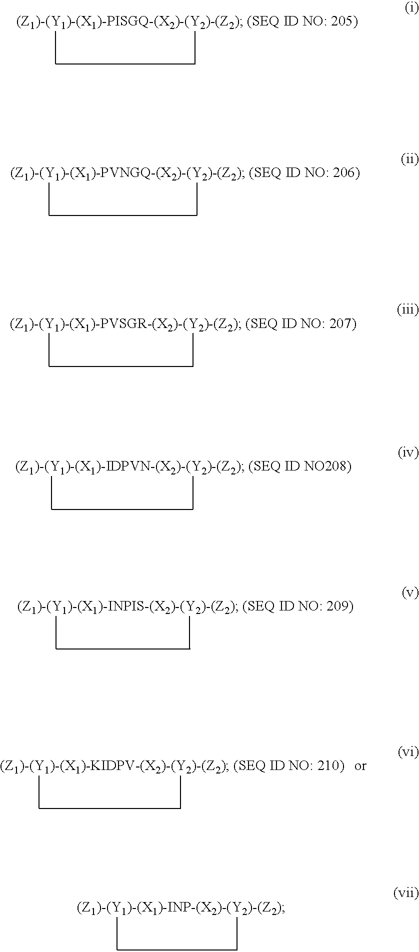

wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein a covalent disulfide bond is formed between residues Y₁ and Y₂ which may be modified as described herein; and wherein Z₁ and Z₂ are optional, and if present, are independently selected from the group consisting of amino acid residues and combinations thereof in which the residues are linked by peptide bonds.

d. Non-Classical Cadherin CARs

Other CAR sequences useful in modified cyclic peptides of the invention include those capable of modulating non-classical cadherins, such as OB-cadherin and VE-cadherin. Illustrative non-classical cadherin CAR sequence useful in the present invention include those described in US Patent Publication Nos. 2005/0215482; 2005/0222037; and 2005/0203025, the contents of which are incorporated herein by reference in their entireties.

Illustrative examples of non-classical cadherin CAR sequences which may be used in modified cyclic peptides of the invention have the formula:

(SEQ ID NO:211)

Aaa-Phe-Baa-Ile/Leu/Val-Asp/Asn/Glu-Caa-Daa-Ser/
Thr/Asn-Gly

wherein Aaa, Baa, Caa and Daa are independently selected amino acid residues; Ile/Leu/Val is an amino acid that is selected from the group consisting of isoleucine, leucine and valine, Asp/Asn/Glu is an amino acid that is selected from the group consisting of aspartate, asparagine and glutamate; and Ser/Thr/Asn is an amino acid that is selected from the group consisting of serine, threonine or asparagine. The non-classical cadherin CAR sequence generally consist of at least three consecutive amino acid residues, and preferably at least five consecutive amino acid residues, of a non-classical cadherin wherein the consecutive amino acids are present within a region of the non-classical cadherin having the formula recited above. Other agents may comprise at least nine consecutive amino acid residues of a non-classical cadherin, wherein the nine consecutive amino acid residues comprise a region having a formula as recited above.

Within particular embodiments, the CAR is present within a cyclic peptide that is modified according to the present invention, and the cyclic peptide has the formula:

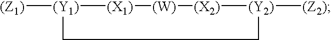

wherein W is a tripeptide selected from the group consisting of EEY, DDK, EAQ, DAE, NEN, ESE, DSG, DEN, EPK, DAN, EEF, NDV, DET, DPK, DDT, DAN, DKF, DEL, DAD, NNK, DLV, NRD, DPS, NQK, NRN, NKD, EKD, ERD, DPV, DSV, DLY, DSN, DSS, DEK, NEK; RAL, YAL, YAT, FAT and YAS wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein a covalent disulfide bond is formed between residues Y₁ and Y₂, which is modified as described herein; and wherein Z₁ and Z₂ are optional, and if present, are independently selected from the group consisting of amino acid residues and combinations thereof in which the residues are linked by peptide bonds.

Particular OB-cadherin CAR sequence may comprise: (a) one or more sequences selected from the group consisting of DDK, IDDK (SEQ ID NO:212) DDKS (SEQ ID NO:213), VIDDK (SEQ ID NO:214), IDDKS (SEQ ID NO:215), VIDDKS (SEQ ID NO:216), DDKSG (SEQ ID NO:217), IDDKSG (SEQ ID NO:218), VIDDKSG (SEQ ID NO:219), FVIDDK (SEQ ID NO:220), FVIDDKS (SEQ ID NO:221), FVIDDKSG (SEQ ID NO:222), IFVIDDK (SEQ ID NO:223), IFVIDDKS (SEQ ID NO:224), IFVIDDKSG (SEQ ID NO:225), EEY, IEEY (SEQ ID NO:226), EEYT (SEQ ID NO:227), VIEEY (SEQ ID NO:228), IEEYT (SEQ ID NO:229), VIEEYT (SEQ ID NO:230), EEYTG (SEQ ID NO:231), IEEYTG (SEQ ID NO:232), VIEEYTG (SEQ ID NO:233), FVIEEY (SEQ ID NO:234), FVIEEYT (SEQ ID NO:235), FVIEEYTG (SEQ ID NO:236), FFVIEEY (SEQ ID NO:237), FFVIEEYT (SEQ ID NO:238), FFVIEEYTG (SEQ ID NO:239), EAQ, VEAQ (SEQ ID NO:240), EAQT (SEQ ID NO:241), SVEAQ (SEQ ID NO:242), VEAQT (SEQ ID NO:243), SVEAQT (SEQ ID NO:244), EAQTG (SEQ ID NO:245), VEAQTG (SEQ ID NO:246), SVEAQTG (SEQ ID NO:247), FSVEAQ (SEQ ID NO:248), FSVEAQT (SEQ ID NO:249), FSVEAQTG (SEQ ID NO:250), YFSVEAQ (SEQ ID NO:251), YFSVEAQT (SEQ ID NO:252) and YFSVEAQTG (SEQ ID NO:253); or (b) an analogue of any of the foregoing sequences that differs in one or more substitutions, deletions, additions and/or insertions such that that ability of the analogue to modulate an OB-cadherin-mediated function is not substantially diminished. For example, the agent may comprise a CAR sequence having the sequence N-Ac-IFVIDDKSG-NH₂ (SEQ ID NO:225), N-Ac-FFVIEEYTG-NH₂ (SEQ ID NO:239) or N-Ac-YFSVEAQTG-NH₂ (SEQ ID NO:253).

Particular cadherin-5 (also known as VE-cadherin) CARs useful in modified cyclic peptides of the invention can comprise, for example, (a) one or more sequences selected from the group consisting of DAE, VDAE (SEQ ID NO:254), DAET (SEQ ID NO:255), RVDAE (SEQ ID NO:256), VDAET (SEQ ID NO:257), RVDAET (SEQ ID NO:258), DAETG (SEQ ID NO:259), VDAETG (SEQ ID NO:260), RVDAETG (SEQ ID NO:261), FRVDAE (SEQ ID NO:262), FRVDAET (SEQ ID NO:263), FRVDAETG (SEQ ID NO:264), VFRVDAE (SEQ ID NO:265), VFRVDAET (SEQ ID NO:266) and VFRVDAETG (SEQ ID NO:267); or (b) an analogue of any of the foregoing sequences that differs in one or more substitutions, deletions, additions and/or insertions such that that ability of the analogue to modulate a cadherin-5-mediated function is not substantially diminished. For example, the agent may comprise the sequence N-Ac-VFRVDAETG-NH₂ (SEQ ID NO:267).

d. Other CAR Sequences

In addition to the illustrative CAR sequences discussed above, it will be understood that other CAR sequences have been identified and may similarly be used in the context of the present invention. For example, illustrative occludin CAR sequences useful in the present invention are described in U.S. Pat. Nos. 6,248,864; 6,110,747; 6,797,807; and 6,310,177. Illustrative claudin CAR sequences useful in the present invention are described in U.S. Pat. Nos. 6,756,356; 6,723,700; and 6,830,894. Illustrative JAM CAR sequences are described in U.S. Pat. No. 6,391,855. All such references, as well as the CAR sequences and cyclic peptides therein, are incorporated herein by reference in their entireties.

The size of a cyclic peptide ring of the present invention generally ranges from about 3 to 50 amino acid residues, but smaller cyclic peptides from about 5 to 15 residues, or 5 to 10 residues, may be preferred in certain embodiments. Further, additional residue(s) may be present on the N-terminal and/or C-terminal side of a CAR sequence, and may be derived from sequences that flank a native CAR sequence, with or without amino acid substitutions and/or other modifications. Alternatively, additional residues present on one or both sides of the CAR sequence(s) may be unrelated to an endogenous sequence (e.g., residues that facilitate cyclization, purification or other manipulation and/or residues having a targeting or other function).

Further, cyclic peptides may comprise an analogue or mimetic of a CAR sequence described or referenced. An analogue generally retains at least 50% identity to a native cadherin CAR sequence, and modulates a cadherin-mediated function as described herein. Such analogues preferably contain at least three consecutive residues of, and more preferably at least five consecutive residues of, a CAR sequence. An analogue may contain any of a variety of amino acid substitutions, additions, deletions and/or modifications (e.g., side chain modifications). Preferred amino acid substitutions are conservative. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A mimetic is a non-peptidyl compound that is conformationally similar to a CAR sequence, such that it modulates a cadherin-mediated function. Such mimetics may be designed based on techniques that evaluate the three dimensional structure of the peptide. For example, Nuclear Magnetic Resonance spectroscopy (NMR) and computational techniques may be used to determine the conformation of a CAR sequence. NMR is widely used for structural analyses of both peptidyl and non-peptidyl compounds. Nuclear Overhauser Enhancements (NOE's), coupling constants and chemical shifts depend on the conformation of a compound. NOE data provides the interproton distance between protons through space and can be used to calculate the lowest energy conformation for the CAR sequence. This information can then be used to design mimetics of the preferred conformation. Linear peptides in solution exist in many conformations. By using conformational restriction techniques it is possible to fix the peptide in the active conformation. Conformational restriction can be achieved by i) introduction of an alkyl group such as a methyl which sterically restricts free bond rotation; ii) introduction of unsaturation which fixes the relative positions of the terminal and geminal substituents; and/or iii) cyclization, which fixes the relative positions of the sidechains. Mimetics may be synthesized where one or more of the amide linkages has been replaced by isosteres, substituents or groups which have the same size or volume such as —CH₂NH—, —CSNH—, —CH₂S—, —CH═CH—, —CH₂CH₂—, —CONMe- and others. These backbone amide linkages can also be part of a ring structure (e.g., lactam). Mimetics may be designed where one or more of the side chain functionalities of the CAR sequence are replaced by groups that do not necessarily have the same size or volume, but have similar chemical and/or physical properties which produce similar biological responses. Other mimetics may be small molecule mimics, which may be identified from small molecule libraries, based on the three-dimensional structure of the CAR sequence. It should be understood that, within embodiments described below, an analogue or mimetic may be substituted for a CAR sequence.

### Preparation of Cyclic Peptides

The preparation and characterization of cyclic peptides comprising CAR sequences is well known and is illustratively described in the references incorporated herein. For example, for certain embodiments, to facilitate the preparation of cyclic peptides having a desired specificity, nuclear magnetic resonance (NMR) and computational techniques may be used to determine the conformation of a peptide that confers a known specificity. NMR is widely used for structural analysis of molecules. Cross-peak intensities in nuclear Overhauser enhancement (NOE) spectra, coupling constants and chemical shifts depend on the conformation of a compound. NOE data provide the interproton distance between protons through space and across the ring of the cyclic peptide. This information may be used to facilitate calculation of the low energy conformations for the CAR sequence. Conformation may then be correlated with tissue specificity to permit the identification of peptides that are similarly tissue specific or have enhanced tissue specificity.

Cyclic peptides as described herein may comprise residues of L-amino acids, D-amino acids, or any combination thereof. Amino acids may be from natural or non-natural sources, provided that at least one amino group and at least one carboxyl group are present in the molecule; α- and β-amino acids are generally preferred. The 20 L-amino acids commonly found in proteins are identified herein by the conventional three-letter or one-letter abbreviations indicated in Table 1, and the corresponding D-amino acids are designated by a lower case one letter symbol. Modulating agents and cyclic peptides may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation, and the like). Certain derivatives include amino acids having an N-acetyl group (such that the amino group that represents the N-terminus of the linear peptide prior to cyclization is acetylated) and/or a C-terminal amide group (i.e., the carboxy terminus of the linear peptide prior to cyclization is amidated). Residues other than common amino acids that may be present with a cyclic peptide include, but are not limited to, penicillamine, β,β-tetramethylene cysteine, β,β-pentamethylene cysteine, β-mercaptopropionic acid, β,β-pentamethylene-β-mercaptopropionic acid, 2-mercaptobenzene, 2-mercaptoaniline, 2-mercaptoproline, ornithine, diaminobutyric acid, α-aminoadipic acid, m-aminomethylbenzoic acid and α,β-diaminopropionic acid.

TABLE 1
Amino acid one-letter and three-letter abbreviations

| | | |
|---|---|---|
| A | Ala | Alanine |
| R | Arg | Arginine |
| D | Asp | Aspartic acid |
| N | Asn | Asparagine |
| C | Cys | Cysteine |
| Q | Gln | Glutamine |
| E | Glu | Glutamic acid |
| G | Gly | Glycine |
| H | His | Histidine |
| I | Ile | Isoleucine |
| L | Leu | Leucine |
| K | Lys | Lysine |
| M | Met | Methionine |
| F | Phe | Phenylalanine |
| P | Pro | Proline |
| S | Ser | Serine |
| T | Thr | Threonine |
| W | Trp | Tryptophan |
| Y | Tyr | Tyrosine |
| V | Val | Valine |

Cyclic peptides as described herein may be synthesized by methods well known in the art, including recombinant DNA methods and chemical synthesis. Chemical synthesis may generally be performed using standard solution phase or solid phase peptide synthesis techniques, in which a peptide linkage occurs through the direct condensation of the α-amino group of one amino acid with the α-carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (see Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solid phase and solution synthesis requires consideration of main chain and side chain protecting groups and activation method. In addition, careful segment selection is necessary to minimize racemization during segment condensation. Solubility considerations are also a factor.

Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc. 85:2149, 1963, which involves assembling a linear peptide chain on a resin support using protected amino acids. Solid phase peptide synthesis typically utilizes either the Boc or Fmoc strategy. The Boc strategy uses a 1% cross-linked polystyrene resin. The standard protecting group for α-amino functions is the tert-butyloxycarbonyl (Boc) group. This group can be removed with dilute solutions of strong acids such as 25% trifluoroacetic acid (TFA). The next Boc-amino acid is typically coupled to the amino acyl resin using dicyclohexylcarbodiimide (DCC). Following completion of the assembly, the peptide-resin is treated with anhydrous HF to cleave the benzyl ester link and liberate the free peptide. Side-chain functional groups are usually blocked during synthesis by benzyl-derived blocking groups, which are also cleaved by HF. The free peptide is then extracted from the resin with a suitable solvent, purified and characterized. Newly synthesized peptides can be purified, for example, by gel filtration, HPLC, partition chromatography and/or ion-exchange chromatography, and may be characterized by, for example, mass spectrometry or amino acid sequence analysis. In the Boc strategy, C-terminal amidated peptides can be obtained using benzhydrylamine or methylbenzhydrylamine resins, which yield peptide amides directly upon cleavage with HF.

In the procedures discussed above, the selectivity of the side-chain blocking groups and of the peptide-resin link depends upon the differences in the rate of acidolytic cleavage. Orthogonal systems have been introduced in which the side-chain blocking groups and the peptide-resin link are completely stable to the reagent used to remove the α-protecting group at each step of the synthesis. The most common of these methods involves the 9-fluorenylmethyloxycarbonyl (Fmoc) approach. Within this method, the side-chain protecting groups and the peptide-resin link are completely stable to the secondary amines used for cleaving the N-α-Fmoc group. The side-chain protection and the peptide-resin link are cleaved by mild acidolysis. The repeated contact with base makes the Merrifield resin unsuitable for Fmoc chemistry, and p-alkoxybenzyl esters linked to the resin are generally used. Deprotection and cleavage are generally accomplished using TFA.

Those of ordinary skill in the art will recognize that, in solid phase synthesis, deprotection and coupling reactions must go to completion and the side-chain blocking groups must be stable throughout the entire synthesis. In addition, solid phase synthesis is generally most suitable when peptides are to be made on a small scale.

Acetylation of an N-terminal residue can be accomplished, for example, by reacting the final peptide with acetic anhydride before cleavage from the resin. C-amidation is accomplished using an appropriate resin such as methylbenzhydrylamine resin using the Boc technology.

Following synthesis of a linear peptide, with or without N-acetylation and/or C-amidation, cyclization may be achieved by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. This method suffers from the disadvantage of being slow but has the advantage of only producing H₂O as a side product. Alternatively, strong oxidizing agents such as I₂ and K₃Fe(CN)₆ can be used to form disulfide linkages. Those of ordinary skill in the art will recognize that care must be taken not to oxidize the sensitive side chains of Met, Tyr, Trp or His. Cyclic peptides produced by this method require purification using standard techniques, but this oxidation is applicable at acid pHs.

Oxidizing agents also allow concurrent deprotection/oxidation of suitable S-protected linear precursors to avoid premature, nonspecific oxidation of free cysteine. DMSO, unlike I₂ and K₃Fe(CN)₆, is a mild oxidizing agent which does not cause oxidative side reactions of the nucleophilic amino acids mentioned above. DMSO is miscible with H₂O at all concentrations, and oxidations can be performed at acidic to neutral pHs with harmless byproducts. Methyltrichlorosilane-diphenylsulfoxide may alternatively be used as an oxidizing agent, for concurrent deprotection/oxidation of S-Acm, S-Tacm or S-t-Bu of cysteine without affecting other nucleophilic amino acids. There are no polymeric products resulting from intermolecular disulfide bond formation.

Suitable thiol-containing residues for use in such oxidation methods include, but are not limited to, cysteine, β,β-dimethyl cysteine (penicillamine or Pen), β,β-tetramethylene cysteine (Tmc), β,β-pentamethylene cysteine (Pmc), β-mercaptopropionic acid (Mpr), β,β-pentamethylene-β-mercaptopropionic acid (Pmp), 2-mercaptobenzene, 2-mercaptoaniline and 2-mercaptoproline.

As noted above, a modulating agent may consist entirely of one or more cyclic peptides, or may contain additional peptide and/or non-peptide sequences. Peptide portions may be synthesized as described above or may be prepared using recombinant methods. Within such methods, all or part of a modulating agent can be synthesized in living cells, using any of a variety of expression vectors known to those of ordinary skill in the art to be appropriate for the particular host cell. Suitable host cells may include bacteria, yeast cells, mammalian cells, insect cells, plant cells, algae and other animal cells (e.g., hybridoma, CHO, myeloma). The DNA sequences expressed in this manner may encode portions of an endogenous cadherin or other adhesion molecule. Such sequences may be prepared based on known cDNA or genomic sequences (see Blaschuk et al., J. Mol. Biol. 211:679-682, 1990), or from sequences isolated by screening an appropriate library with probes designed based on the sequences of known cadherins. Such screens may generally be performed as described in Sambrook et al., Molecular Cloning: A Laboratory Manual, Cold Spring Harbor Laboratories, Cold Spring Harbor, N.Y., 1989 (and references cited therein). Polymerase chain reaction (PCR) may also be employed, using oligonucleotide primers in methods well known in the art, to isolate nucleic acid molecules encoding all or a portion of an endogenous adhesion molecule. To generate a nucleic acid molecule encoding a peptide portion of a modulating agent, an endogenous sequence may be modified using well known techniques. For example, portions encoding one or more CAR sequences may be joined, with or without separation by nucleic acid regions encoding linkers, as discussed above. Alternatively, portions of the desired nucleic acid sequences may be synthesized using well known techniques, and then ligated together to form a sequence encoding a portion of the modulating agent.

The chemical modifications described herein can be made to disulfide-containing cyclic peptides using known and established synthetic chemistry techniques. Although detailed descriptions of the synthetic methodologies used to produce particular modified cyclic peptides of the invention are set forth in the Examples below, one skilled in the art would understand and recognize that any of a number of synthetic approaches may be employed while achieving the same or similar result. Accordingly, the discussion below is intended to provide general illustrative guidance for certain classes of disulfide modifications of the invention, but is not limiting in this respect.

For example, oxidation of a cyclic peptide to form a thiosulfonate can be achieved via treatment with an appropriate oxidizing agent, such as mCPBA.

Synthesis of compounds in which the sulfur atoms of the disulfide bond are replaced may be performed using resin based peptide synthesis methods. For example, a typical synthesis is outlined below for an analog such as 3 in which an amide bond replaces the disulfide.

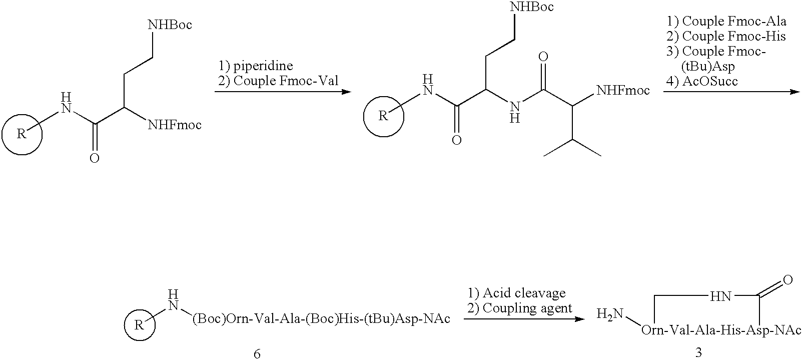

Attachment of ε-N(Boc)-α-N(Fmoc)ornithine to Rink resin can allow for the final liberation of a primary amide group. Deprotection of the Fmoc group followed by standard peptide coupling of protected valine, alanine, histidine and t-Bu-aspartic acid creates the desired pentapeptide. Final capping of the terminal amino group with an acetate group completes the synthesis of the linear peptide 6. Cleavage of the peptide from the solid support under acidic conditions results in simultaneous deprotection of the Boc groups such that the terminal amino group in the ornithine residue, and the terminal acid group in the aspartic acid residue become exposed. Treatment of this liberated amino acid with typical peptide coupling conditions allows the key cyclization to occur to yield 3.

By choosing a protected lysine in place of ornithine, and a protected glutamic acid in place of aspartic acid, and inverting the position of these acid and amide residues, a wide selection of cyclic amide containing peptides can be produced.

In addition, the choice of protecting groups for the histidine and acid/amino groups may be modified in order to allow greater control of the key cyclization steps. The use of Cbz as a protecting group for histidine allows this residue to remain protected during the key cyclization step which may improve the reaction. The key final cyclization is facilitated by the β-, or γ-, turn that the molecule can adopt which should constrain the two coupling ends.

A similar synthetic approach may be used for the synthesis of compounds in which the disulfide link is replaced by an alkyl chain. Use of a non-natural amino acid such as fmoc-allylglycine in place of the ornithine and aspartic acid residues outlined above leads to a peptide such as 7.

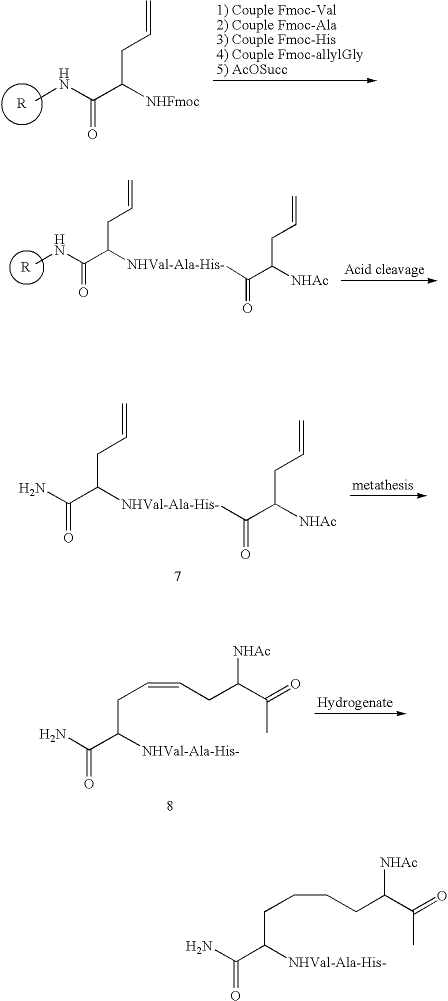

The two olefin functions in 7 should be suitably positioned to participate in a Ruthenium catalyzed Grubbs' metathesis reaction to generate a cyclic compound containing a new olefin 8. This olefinic compound can be hydrogenated to afford the simple alkyl chain. The olefin in compound 8 also provides opportunity to introduce additional solubilizing groups. For example, hydroboration of the olefin followed by oxidative workup yields an alcohol such as 9. Further alkylation of the alcohol with an amine bearing compound such as N,N-dimethyl-2-chloroethylamine leads to a compound with significantly improved water solubility.

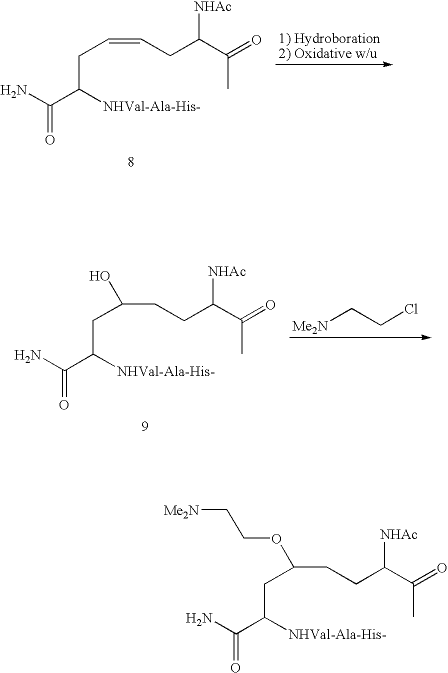

Use of homoallyglycine in place of allylglycine, and various permutations thereof, allows the alkyl chain link in compounds such as 10 to be varied in length to better match the conformation of the parent cyclic peptide 1.

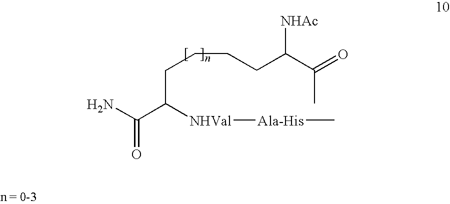

The synthesis of a pegylated cyclic peptide based on 1 can be prepared using the general synthesis outlined for compounds such as 3. Using standard peptide synthesis, the preparation of a protected pentapeptide such as 11 should proceed without incident. This pentapeptide can then be capped with a group that is amenable to pegylation technology. An example is to cap the terminal amine with an activated acetate group such as chloroacetate. Subsequent treatment with PEG methyl ether leads to a desired compound. Subsequent cleavage from the resin and disulfide bond formation gives rise to a pegylated compound. Additional methods for attachment of a PEG group could involve capping the amino group of pentapeptide 11 with a glycine residue and then using the amino group of this unhindered amino acid to react with PEG-acrylates, with PEG-alcohol via an isocyanate or with PEG-mesylate.

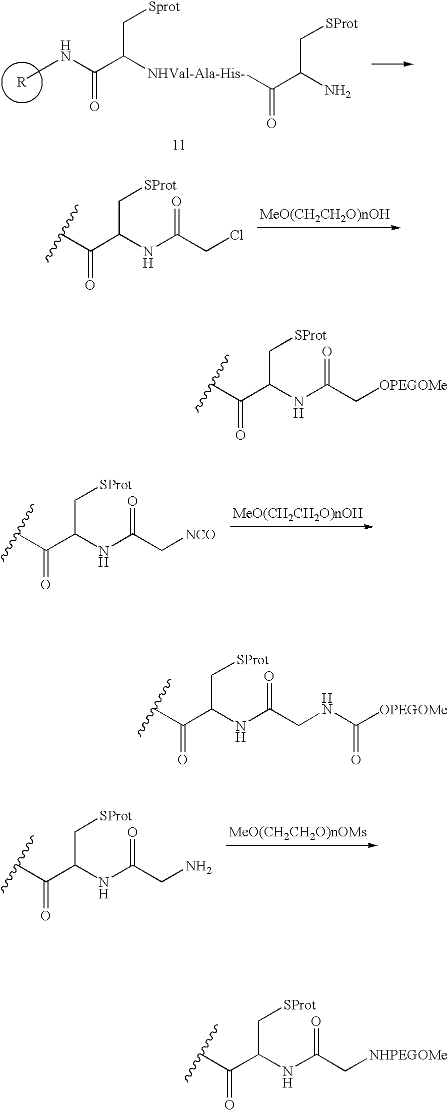

### Pharmaceutical Compositions & Methods of Use

Within further aspects, the present invention provides cell adhesion modulating agents that comprise a modified cyclic peptide as described above. Within specific embodiments, such modulating agents may be linked to one or more of a targeting agent, a drug, a solid support or support molecule, a detectable marker, or other moiety of interest.

The present invention further provides pharmaceutical compositions comprising cyclic peptides and cell adhesion modulating agents as described above, in combination with a pharmaceutically acceptable carrier. Such compositions may further comprise a drug or other compound of interest.

Within further aspects, methods are provided for modulating cell adhesion, comprising contacting a cell, e.g., a cadherin-expressing cell, with a cell adhesion modulating agent as described above.

Within further aspects, methods are provided for modulating cell proliferation, comprising contacting a cell, e.g., a cadherin-expressing cell, with a cell adhesion modulating agent as described above.

Within further aspects, methods are provided for modulating cell migration, comprising contacting a cell, e.g., a cadherin-expressing cell, with a cell adhesion modulating agent as described above.

Within further aspects, methods are provided for modulating cell survival, comprising contacting a cell, e.g., a cadherin-expressing cell, with a cell adhesion modulating agent as described above.

Within related aspects, methods for treating cancer and/or inhibiting metastasis of tumor cells in a mammal are provided, comprising contacting a cell, e.g., a cadherin-expressing cell with, or administering to a mammal afflicted with cancer, a cell adhesion modulating agent as described above.

These and a variety of other methods of using cyclic peptides comprising the CAR sequences described herein are known in the art and/or described in the references cited herein. Modified cyclic peptides of the present invention may be used in all such methods.

Further, as described in co-pending U.S. Provisional Patent Application No. 60/848,624, filed Sep. 27, 2006 (incorporated herein by reference in its entirety) cyclic peptides comprising HAV and other CAR sequences provide synergistic efficacy when used in combination with particular conventional forms of cancer therapies. Accordingly, the modified cyclic peptides of the present invention are useful in all such combination therapies described therein. For example, there is provided a method for the treatment of a cancer comprising administering to a subject in need thereof at least one modified cyclic peptide of the invention and at least one anticancer alkylating agent. The alkylating agent may be selected, for example, from agents such as mechlorethamine, cyclophosphamide, ifosfamide, trofosfamide, melphalan (L-sarcolysin), chlorambucil, hexamethylmelamine, thiotepa, busulfan, carmustine (BCNU), streptozocin (streptozotocin), dacarbazine (DTIC; dimethyltriazenoimid-azolecarboxamide) and temozolomide. In one preferred embodiment, the agent is melphalan.

According to another aspect of the invention, there is provided a method for the treatment of a cancer comprising administering to a subject in need thereof at least one modified cyclic peptide of the invention and at least one anticancer antimetabolite, such as an agent selected from pyrimidine analogs and purine analogs. In a particular embodiment, the anticancer antimetabolite is selected from the group consisting of fluorouracil, 5-fluorouracil, floxuridine (fluoride-oxyuridine; FUdR), capecitabine, pemetrexed, cytarabine (cytosine arabinoside), gemcitabine, mercaptopurine (6-mercaptopurine; 6-MP) and thioguanine.

According to another aspect of the invention, there is provided a method for the treatment of a cancer comprising administering to a subject in need thereof at least one modified cyclic peptide of the invention and at least one anticancer natural product, such as an agent selected from vinca alkaloids, taxanes, epipodophylltoxins, camptothecins antibiotics, enzymes, biological response modifiers and immunostimulators. In one embodiment, the anticancer natural product is selected from the group consisting of docataxel, etoposide, teniposide; topotecan, irinotecan, dactinomycin (actinomycin D), daunorubicin (daunomycin; rubidomycin), doxorubicin, bleomycin, L-Asparaginase, interferon-alfa and interleukin 2. In a particular embodiment, the natural product anticancer agent is not taxol or a vinca alkaloid.

In another aspect, the invention provides a method for the treatment of a cancer comprising administering to a subject in need thereof at least one modified cyclic peptide of the invention and at least one anticancer antibiotic, such as an agent selected from eiprubicin, idarubicin and liposomal doxorubicin.

In another aspect, the invention provides a method for the treatment of a cancer comprising administering to a subject in need thereof at least one modified cyclic peptide of the invention and at least one agent selected from the group consisting of plantinum compounds, anthracenediones, methylhydrazine derivatives, adrenocortical suppressants, tyrosine kinase inhibitors, multi-targeted kinase inhibitors, adrenocorticosteroids, estrogens, progestins, aromatase inhibitors, antiestrogens, antitumor antibodies and radiation therapy. In one embodiment, the platinum compound is selected from the group consisting of cisplatin (cis-DDP), carboplatin and oxaliplatin.

In a more particular embodiment, the anthracenedione is mitoxantrone. In another embodiment, the methylhydrazine derivative is N-methylhydrazine (MIH). In yet another embodiment, the adrenocortical suppressant is selected from the group consisting of mitotane and aminoglutethimide. In another embodiment, the tyrosine kinase inhibitor is selected from the group consisting of imatinib, erlotinib and gefitinib. In another embodiment, the multi-targeted kinase inhibitor is selected from the group consisting of sunitinib, sorafanib and dasatinib. In another embodiment, the adrenocorticosteriods is selected from the group consisting of prednisone and prednisolone. In another embodiment, the estrogen is diethylstilbestrol. In another embodiment, the progestin is megestrol acetate. In another embodiment, the aromatase inhibitor is selected from the group consisting of exemestane and letrozole. In another embodiment, the antiestrogen is tamoxifen. In another embodiment, the anticancer antibody is selected from the group consisting of bevacizumab, rituximab, cetuximab, panitumomab and ¹³¹I-tositumomab.

In the methods of the invention, cyclic peptides and modulating agents may delivered by essentially any administration approach suitable to a given indication and compatible with the delivery of modulating agents provided herein. Exemplary delivery approaches are well known and established in the art, illustrative examples of which are described in the references cited herein.

The following Examples are offered by way of illustration and not by way of limitation.

### EXAMPLES

### Example 1

### Synthesis of Modified Cyclic Peptides

Described below are the experimental procedures used to synthesize illustrative modified cyclic peptides of the invention, referred to herein as ADH100703, ADH100707, ADH100710, ADH100711, ADH100713, ADH100714, ADH100715, ADH100716. These modified cyclic peptides are based on the ADH-1 compound (N-Ac-CHAVC-NH2). While the embodiments exemplified herein relate in particular to ADH-1, however, it will be understood that the modifications are similarly applicable to cyclic peptides containing other CAR sequences as discussed above, based upon the structural similarity of the compounds and further based upon their functional similarities in modulating cell adhesion.

Abbreviations: Cys: Cysteine; Val: Valine; Ala: Alanine; His: Histidine; Pen: Penicillamine; Glu: glutamic acid; Lys: Lysine; Dab: diaminobutyric acid; I-Phe: 4-iodophenylalanine; Fmoc: 9-fluorenylmethoxy carbonyl; Boc: tertButoxy carbonyl; Acm: acetamido methyl; Trt: trytil; Alloc: allyloxycarbonyl; HOBt: 1-Hydroxy benzotriazole; DIC: diisopropyl carbodiimide; PyBOP: (Benzotriazol-1-yloxy)tripyrrolidinaphosphonium hexafluorophosphate; HOAt: 1-Hydroxy-7azabenzotriazole; mPEG₃₅₀: methoxypolyethyleneglycol 350 (average molecular weight: 350 Da); dppf: 1,1′-Bis(diphenylphosphino)ferrocene; Pd₂ dba₃: Tris(dibenzylidene-acetone)dipalladium; Pd(PPh₃)₄: Tetrakis (triphenylphosphine) palladium; DMF: dimethylformamide; CH₂Cl₂: dichloromethane; MeOH: Methanol; and

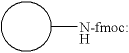

Knorr resin.

All temperatures are given in degrees Centigrade. Reagents were purchased from commercial sources or prepared following literature procedures.

Unless otherwise noted, HPLC purification was performed using a 50 mm Varian Dynamax HPLC 21.4 mm Microsorb Guard-8 C₁₈ column, Dyonex Chromeleon operating system coupled with a Varian Prostar 320 UV-vis detector (210 nm) and a Sedex55 ELS detector. Conditions: Solvent A: H₂O/0.1% TFA; Solvent B: Acetonitrile/0.1% TFA. The gradient holds at 0% B for a minute and goes to 50% B over 4 minutes. The column was washed with 50% B over 1.5 minutes. The gradient goes to 0% B over 0.5 minutes and holds at 0% B for 1 minute. Total run time was 8 minutes. The resulting fractions were analyzed, combined as appropriate, evaporated under reduced pressure and lyophilized to provide purified material.

Proton magnetic resonance (¹H NMR) spectra were recorded on either a Varian INOVA 400 MHz (¹H) NMR spectrometer, Varian INOVA 500 MHz (¹H) NMR spectrometer, Bruker ARX 300 MHz (¹H) NMR spectrometer, Bruker DPX 400 MHz (¹H) NMR spectrometer, or a Bruker DRX 500 MHz (1H) NMR spectrometer. All spectra were determined in the solvents indicated. Although chemical shifts are reported in ppm downfield of tetramethylsilane, they are referenced to the residual proton peak of the respective solvent peak for ¹H NMR. Interproton coupling constants are reported in Hertz (Hz).

Analytical HPLC was performed using a Supelco discovery C₁₈ 15 cm×4.6 mm/5 μm column coupled with an Agilent 1050 series VWD UV detector at 210 nm. Conditions: Solvent A: H₂O/0.1% TFA; Solvent B: acetonitrile/0.1% TFA. The gradient holds at 2% B for 3 minutes and goes to 50% B over 10 minutes. The gradient goes to 100% B over 1 minute, holds at 100% B for 3 minutes and goes to 2% B in 2 minutes and holds at 2% B for 3 minutes.

LCMS spectra were obtained using a ThermoFinnigan AQA MS ESI instrument utilizing a Phenomenex Aqua 5 micron C₁₈ 125 Å 50×4.60 mm column. The initial gradient was 5% MeOH: 1% CH₃CN, 0.1% formic acid in H₂O which was ramped up to 100% MeOH over 5 minutes. 100% MeOH was maintained for 2 minutes before it was re-equilibrated to the initial starting gradient. The spray setting for the MS probe was at 350 μL/min with a cone voltage at 25 mV and a probe temperature at 450° C. The spectra were recorded using ELS detection.

The commercially available methoxypolyethylene derivative (mPEG₃₅₀, Aldrich) is comprised of polymers with ethylene glycol units (form 3 to 11) with an average molecular weight of 350 Da. The mass reported was the mass of the centroid of the envelope of masses (±44 Da) recorded by the instrument.

The resin used was the Knorr resin. The loading on this resin was 0.75 mmol/g.

Illustrative Solid Phase Synthesis Procedures:

A. Kaiser Test: A small amount of thoroughly washed beads (20-50) were transferred to a 10 mL disposable culture tube. 3 drops of Phenol in Ethanol (80%), 3 drops of KCN in pyridine and Ninhydrin (6% in ethanol) are sequentially added. The tube was heated with a heat gun for ˜1 min. Blue color indicates presence of free amines.

Amino acid coupling: The resin in a peptide synthesis vessel (Chemglass CG1862) was gently shaken with a 20% solution of piperidine in DMF (approx. 3 bed volumes). After 10 minutes, the reaction was filtered and the beads were thoroughly washed with DMF (approx. 3 bed volumes). The piperidine deprotection and the DMF wash were repeated an additional 3 times.

A round bottom flask was charged with the Fmoc-protected amino acid (2 equiv) and DMF (minimum solubilizing amount). HOBt (2.0 equiv) and DIC (2.5 equiv) were sequentially added to the reaction. After 20 minutes of stirring, the reaction mixture was added to the deprotected beads and the peptide synthesis vessel was gently shaken for 4 hours. The reaction was filtered and the beads were thoroughly washed with DMF (3 bed volumes), CH₂Cl₂ (3 bed volumes), MeOH (3 bed volumes), CH₂Cl₂ (3 bed volumes) and MeOH (3 bed volumes). The beads were dried under vacuum. The reaction completion was monitored by Kaiser Test.

B. Acetate Capping: DMF (3 bed volumes) was added to the peptide synthesis vessel containing the deprotected beads. Acetic anhydride (4.0 equiv) and triethylamine (4.0 equiv) were sequentially added. The reaction was shaken gently for ˜30 minutes. The reaction was filtered and the beads were thoroughly washed with DMF (3 bed volumes), CH₂Cl₂ (3 bed volumes), MeOH (3 bed volumes), CH₂Cl₂ (3 bed volumes) and MeOH (3 bed volumes). The beads were dried under vacuum. The reaction completion was monitored by Kaiser Test.

Cleavage from Resin: A freshly made solution of TFA/H₂O/Triisopropylsilane (95/2.5/2.5) (40 mL/g of resin) was added to the peptide synthesis vessel containing thoroughly dried beads. The reaction was gently stirred for 2 hrs and filtered. The beads were washed with 3 bed volumes of TFA. These operations were repeated an additional time.

The combined filtrates were evaporated to dryness under reduced pressure. The peptide was precipitated out using diisopropylether. The white solid was thoroughly dried under high vacuum.

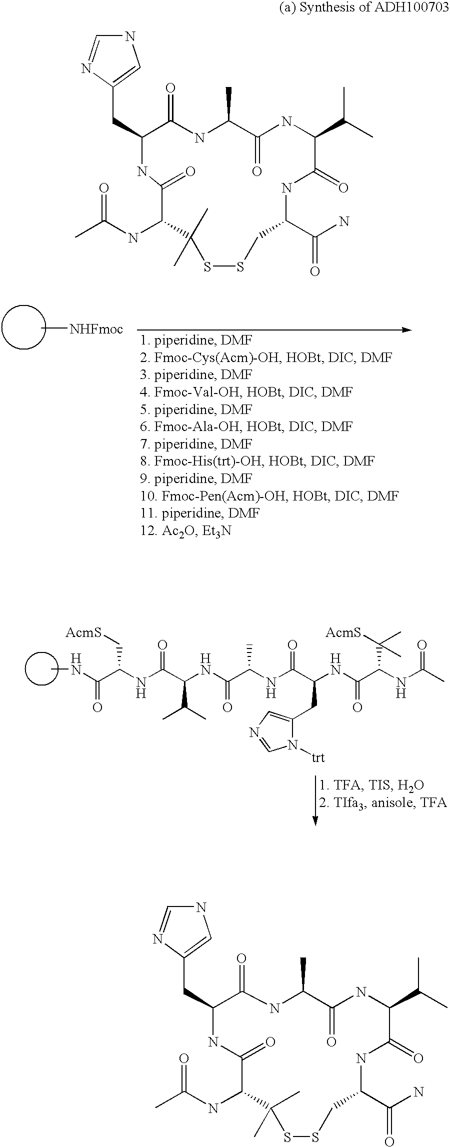

### 4R, 7S, 10S, 13S, 16R, 16-Acetylamino-13-(1H-imidazol-4-ylmethyl)-7-isopropyl-10,17,17-trimethyl-6,9,12,15-tetraoxo-1,2-dithia-5,8,11,14-tetraazacycloheptadecane-4-carboxylic acid amide

Fmoc-Cys(Acm)-OH, Fmoc-Val-OH, Fmoc-Ala-OH, Fmoc-His(trt)-OH and Fmoc-Pen(Acm)-OH were sequentially coupled to 1.5 g of Knorr resin using standard solid phase techniques. The resin was capped with acetic anhydride using the standard procedure. The crude linear peptide (650 mg) was isolated after cleavage.

A round bottom flask was charged with the peptide (83 mg), anisole (5 mL) and trifluoroacetic acid (95 mL). Thallium triflate (63 mg, 1.2 equiv) was added in one portion at 0° C. under nitrogen. After 1 hr, trifluoroacetic acid was evaporated under reduced pressure. Diisoproylether (10 mL) was added. The crude peptide was filtered, redissolved in MeOH-water and purified by HPLC. The desired material (22 mg) was isolated as the trifluoroacetate salt.

¹H NMR (400 MHz, DEUTERIUM OXIDE) δ ppm 0.90 (d, J=6.8 Hz, 3H) 0.94 (d, J=6.8 Hz, 3H) 1.32 (s, 3H) 1.38 (s, 3H) 1.44 (d, J=7.1 Hz, 3H) 1.98 (s, 3H) 2.14-2.30 (m, 1H) 2.77-2.88 (m, 1H) 3.00-3.13 (m, 1H) 3.20-3.36 (m, 2H) 3.95 (d, J=9.2 Hz, 1H) 4.28-4.41 (m, 2H) 4.62 (dd, J=11.4, 3.8 Hz, 1H) 4.76-4.81 (m, 1H) 7.23 (s, 1H) 8.60 (s, 1H)

LCMS: 599 (MH⁺)

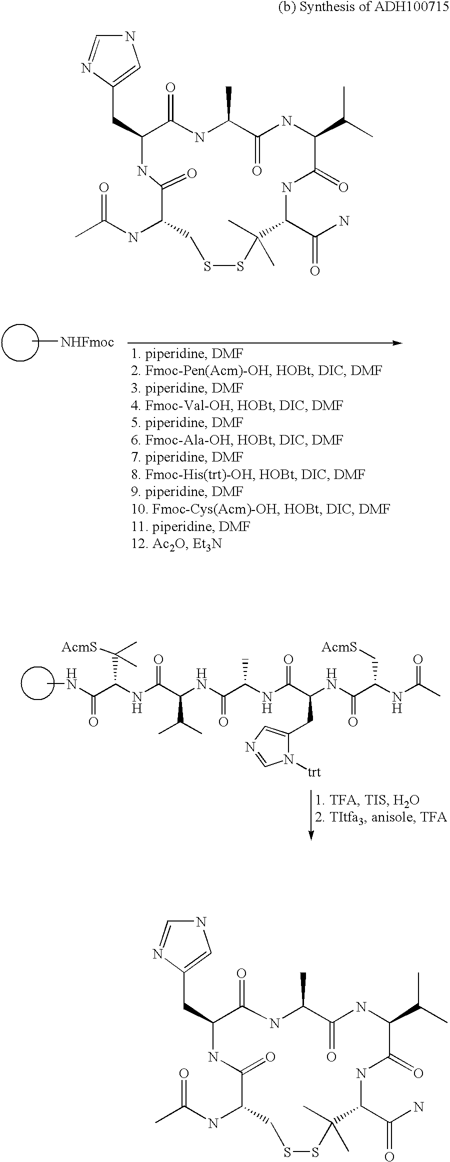

### 4R, 7S, 10S, 13S, 16R, 16-Acetylamino-13-(1H-imidazol-4-ylmethyl)-7-isopropyl-3,3,10-trimethyl-6,9,12,15-tetraoxo-1,2-dithia-5,8,11,14-tetraaza-cycloheptadecane-4-carboxylic acid amide

Fmoc-Pen(Acm)-OH, Fmoc-Val-OH, Fmoc-Ala-OH, Fmoc-His(trt)-OH and Fmoc-Cys(Acm)-OH were sequentially coupled to 650 mg of Knorr resin using standard solid phase techniques. The resin was capped with acetic anhydride using the standard procedure. The crude linear peptide (150 mg) was isolated after cleavage.

A round bottom flask was charged with the peptide (150 mg), anisole (5 mL) and trifluoroacetic acid (95 mL). Thallium triflate (117 mg, 1.2 equiv) was added in one portion at 0° C. under nitrogen. After 1 hr, trifluoroacetic acid was evaporated under reduced pressure. Diisoproylether (10 mL) was added. The peptide was filtered, redissolved in MeOH-water and purified by HPLC. The desired material (37 mg) was isolated as the trifluoroacetate salt.

¹H NMR (400 MHz, DEUTERIUM OXIDE) δ ppm 0.91 (d, J=7.3 Hz, 3H) 0.95 (d, J=7.3 Hz, 3H) 1.28 (s, 3H) 1.44 (d, J=7.1 Hz, 3H) 1.48 (s, 3H) 1.95 (s, 3H) 2.09-2.24 (m, 1H) 2.93 (dd, J=13.8, 9.5 Hz, 1H) 3.13-3.37 (m, 3H) 4.04 (d, J=9.3 Hz, 1H) 4.33-4.43 (m, 2H) 4.48 (t, J=4.1 Hz, 1H) 7.28 (s, 1H) 8.14 (d, J=8.2 Hz, 1H) 8.60 (s, 1H)

LCMS: 599 (MH⁺)

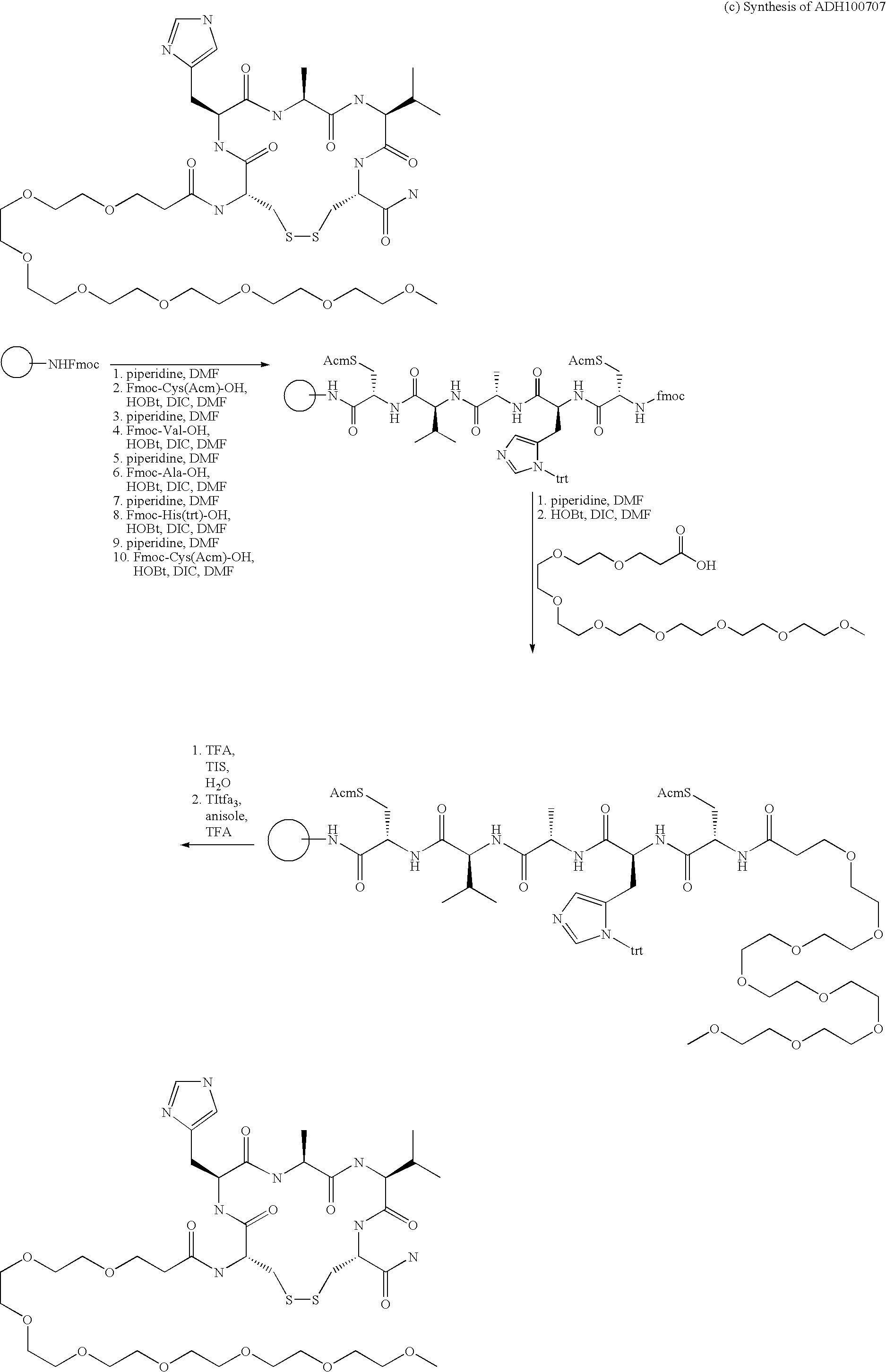

### mPEG₃₅₀ Acid Synthesis: 3-(2-{2-[2-(2-{2-[2-(2-Methoxy-ethoxy)-ethoxy]-ethoxy}-ethoxy)-ethoxy]-ethoxy}-ethoxy)-propionic Acid

A round bottom flask was charged with mPEG₃₅₀ (14.5 g, 3.0 equiv), THF (20 mL) and Sodium (31 mg, 0.1 equiv). Once the sodium had dissolved, tertButyl acrylate (2 mL, 1.0 equiv) was added to the reaction mixture. The reaction was stirred at room temperature for 12 hrs. Dichloromethane (150 mL) was added to the reaction and the organic phase was extracted 3 times with HCl (1N, 30 mL). The organic layer was concentrated under reduced pressure to yield the crude ester. A round bottom flask was charged with the crude ester. Trifluoroacetic acid (50 mL) was added to the reaction at 0° C. under nitrogen. The reaction was stirred one hour, and concentrated under reduced pressure. Toluene (2×50 mL) was used as a chaser to evaporate all trifluoroacetic acid. The crude oil was dissolved in dichloromethane (150 mL) and extracted with HCl (1N, 3×30 mL). The organic layer was concentrated under reduced pressure. A pale brown oil (4 g) was isolated and used directly in the coupling step.

¹H NMR (400 MHz, CHLOROFORM-d) d ppm 2.60 (t, J=6.5 Hz, 2H) 3.38 (s, 3H) 3.51-3.59 (m, 2H) 3.60-3.67 (m, 26H) 3.76 (t, J=6.5 Hz, 2H)

LCMS: 413

### 4R, 7S, 10S, 13S, 16R 13-(1H-Imidazol-4-ylmethyl)-7-isopropyl-16-[3-(2-{2-[2-(2-{2-[2-(2-methoxy-ethoxy)-ethoxy]-ethoxy}-ethoxy)-ethoxy]-ethoxy}-ethoxy)-propionylamino]-10-methyl-6,9,12,15-tetraoxo-1,2-dithia-5,8,11,14-tetraaza-cycloheptadecane-4-carboxylic acid amide

Fmoc-Cys(Acm)-OH, Fmoc-Val-OH, Fmoc-Ala-OH, Fmoc-His(trt)-OH and Fmoc-Cys(Acm)-OH were sequentially coupled to 1 g of Knorr resin using standard solid phase techniques. Solid phase coupling of the mPEG₃₅₀ acid was done using the same procedure as the solid phase amino acid coupling. The crude linear peptide (560 mg) was isolated after cleavage. A round bottom flask was charged with iodine (723 mg, 6.0 equiv), MeOH (600 mL), H₂O (200 mL) and acetamide (168 mg, 10.0 equiv). The peptide in 200 mL of MeOH was added in one portion to the solution. After 12 hrs, ascorbic acid in H₂O (500 mg in 15 mL) was added to quench the remaining iodine. The reaction was concentrated under reduced pressure to ˜70 mL which are purified by HPLC. The desired material (201 mg) was isolated as the trifluoroacetate salt.

¹H NMR (400 MHz, DEUTERIUM OXIDE) δ ppm 0.93 (dd, J=6.6, 3.1 Hz, 6H) 1.45 (d, J=7.1 Hz, 3H) 2.09-2.27 (m, 1H) 2.41-2.62 (m, 2H) 2.86-3.34 (m, 6H) 3.61 (s, 2H) 3.67 (s, 30H) 3.76 (d, J=5.2 Hz, 2H) 4.06 (d, J=8.4 Hz, 1H) 4.31 (q, J=7.1 Hz, 1H) 4.50 (t, J=7.2 Hz, 1H) 4.60-4.71 (m, 2H) 7.30 (s, 1H) 8.62 (s, 1H)

LCMS: 923

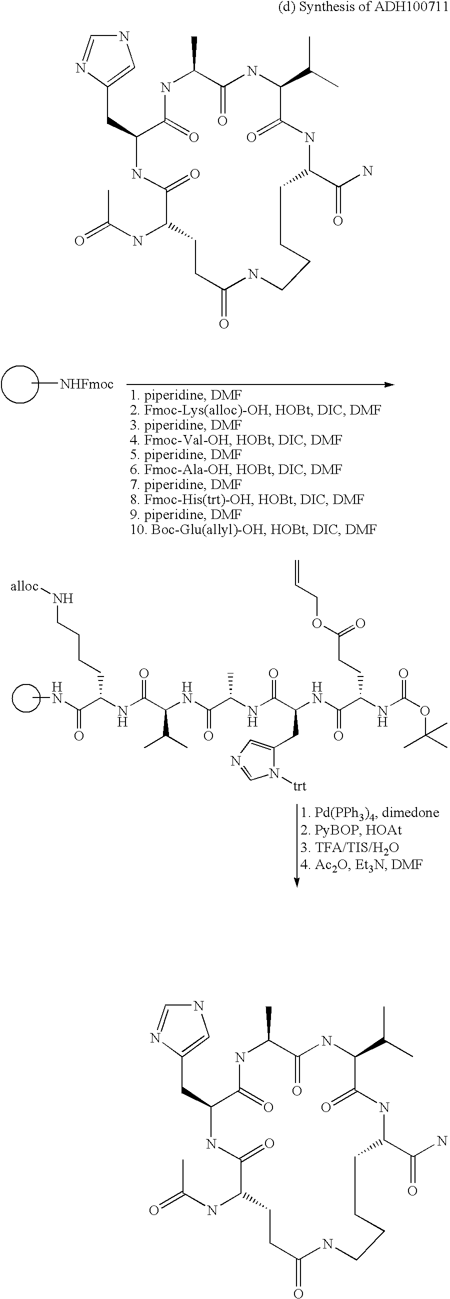

### 2S, 5S, 8S, 11S, 20S 20-Acetylamino-2-(1H-imidazol-4-ylmethyl)-8-isopropyl-5-methyl-3,6,9,17,21-pentaoxo-1,4,7,10,16pentaaza-cyclohenicosane-11-carboxylic acid amide

Fmoc-Lys(alloc)-OH, Fmoc-Val-OH, Fmoc-Ala-OH, Fmoc-His(trt)-OH and Boc-Glu(allyl)-OH were sequentially coupled to 2.0 g of Knorr resin using standard solid phase techniques. A solution of Pd(PPh₃)₄ (3.12 g, 2.0 equiv) in THF (40 mL) and dichloromethane (40 mL) was added to the resin and gently shaken in a peptide synthesis vessel. Dimedone (940 mg, 5.0 equiv) in THF (20 mL) was added to the vessel. The reaction was gently shaken for 3 hrs. The resin was filtered and thoroughly washed with DMF (5 bed volumes), dichloromethane (5 bed volumes), a 5% solution of diethyldithiocarbamate in DMF (5 bed volumes), dichloromethane (5 bed volumes) and MeOH (5 bed volumes). The resin was dried under vacuum. A solution of PyBOP (3.57 g, 5.0 equiv) in DMF (40 mL) was added to the resin. A solution of HOAt (0.6M in DMF, 920 mg, 5.0 equiv) was added. The vessel was gently shaken for 3 hours. The reaction was filtered and the beads were thoroughly washed with DMF (3 bed volumes), CH₂Cl₂ (3 bed volumes), MeOH (3 bed volumes), CH₂Cl₂ (3 bed volumes) and MeOH (3 bed volumes). The beads were thoroughly dried under high vacuum. The peptide was cleaved from the resin using standard conditions. The fully deprotected peptide (360 mg) was isolated after cleavage.

A round bottom flask was charged with the peptide, DMF (10 mL), triethylamine (0.13 mL, 1.5 equiv) and acetic anhydride (0.09 mL, 1.5 equiv). After 2 hrs of stirring, water (20 mL) was added and the crude reaction mixture is purified by HPLC. The desired material (100 mg) was isolated as the trifluoroacetate salt.

¹H NMR (400 MHz, DEUTERIUM OXIDE) δ ppm 0.89 (d, J=6.6 Hz, 3H) 1.03 (d, J=6.6 Hz, 3H) 1.35 (d, J=7.2 Hz, 3H) 1.39-1.54 (m, 4H) 1.62-1.76 (m, 1H) 1.77-1.89 (m, 1H) 1.98 (s, 3H) 2.00-2.12 (m, 3H) 2.35 (q, J=7.7 Hz, 2H) 3.05-3.37 (m, 4H) 3.89-4.00 (m, 1H) 4.17-4.25 (m, 1H) 4.26-4.38 (m, 2H) 4.56 (d, J=6.2 Hz, 1H) 7.32 (s, 1H) 8.62 (s, 1H)

LCMS: 606 (MH⁺)

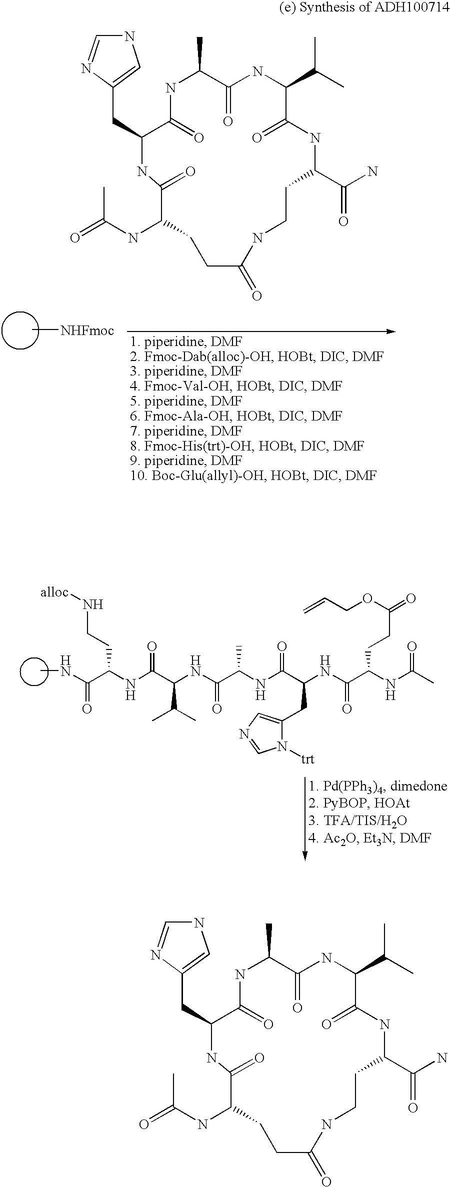

### S, 5S, 8S, 11S, 18S 18-Acetylamino-2-(1H-imidazol-4-ylmethyl)-8-isopropyl-5-methyl-3,6,9,15,19-pentaoxo-1,4,7,10,14pentaaza-cyclononadecane-11-carboxylic acid amide

Fmoc-Dab(alloc)-OH, Fmoc-Val-OH, Fmoc-Ala-OH, Fmoc-His(trt)-OH and Boc-Glu(allyl)-OH were sequentially coupled to 2.0 g of Knorr resin using standard solid phase techniques. A solution of Pd(PPh₃)₄ (3.12 g, 2.0 equiv) in THF (40 mL) and dichloromethane (40 mL) was added to the resin and gently shaken in a peptide vessel. Dimedone (940 mg, 5.0 equiv) in THF (20 mL) was added to the vessel. The reaction was gently shaken for 3 hrs. The resin was filtered and thoroughly washed with DMF (5 bed volumes), dichloromethane (5 bed volumes), a 5% solution of diethyldithiocarbamate in DMF (5 bed volumes), dichloromethane (5 bed volumes) and MeOH (5 bed volumes). The resin was dried under vacuum. A solution of PyBOP (3.57 g, 5.0 equiv) in DMF (40 mL) was added to the resin. A solution of HOAt (0.6M in DMF, 920 mg, 5.0 equiv) was added. The vessel was gently shaken for 3 hours. The reaction was filtered and the beads were thoroughly washed with DMF (3 bed volumes), CH₂Cl₂ (3 bed volumes), MeOH (3 bed volumes), CH₂Cl₂ (3 bed volumes) and MeOH (3 bed volumes). The beads were thoroughly dried under high vacuum. The peptide was cleaved from the resin using standard conditions. The fully deprotected peptide (510 mg) was isolated after cleavage.

A round bottom flask was charged with the peptide, DMF (10 mL), triethylamine (0.13 mL, 1.5 equiv) and acetic anhydride (0.09 mL, 1.5 equiv). After 2 hrs of stirring, water (20 mL) was added and the crude reaction mixture is purified by HPLC. The desired material (190 mg) was isolated as the trifluoroacetate salt.

¹H NMR (400 MHz, DEUTERIUM OXIDE) δ ppm 0.92 (d, J=6.7 Hz, 3H) 0.99 (d, J=6.7 Hz, 3H) 1.40 (d, J=7.1 Hz, 3H) 1.78-1.93 (m, 1H) 1.97 (s, 3H) 1.99-2.14 (m, 4H) 2.33 (t, J=7.7 Hz, 2H) 3.05-3.26 (m, 2H) 3.35 (dd, 1H) 3.46-3.64 (m, 1H) 3.89 (d, J=8.9 Hz, 1H) 4.27 (t, J=6.1 Hz, 1H) 4.32-4.43 (m, 2H) 4.62 (dd, J=9.6, 5.8 Hz, 1H) 7.31 (s, 1H) 8.61 (s, 1H)

LCMS: 578 (MH⁺)

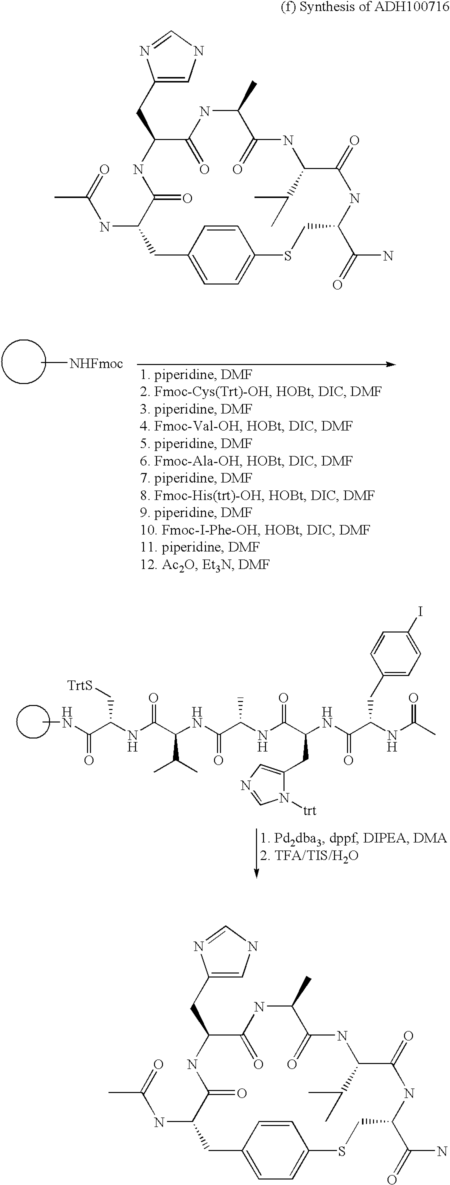

### 4R, 7S, 10S, 13S, 16S 16-Acetylamino-13-(1H-imidazol-4-ylmethyl)-7-isopropyl-10-methyl-6,9,12,15-tetraoxo-2-thia-5,8,11,14-tetraaza-bicyclo[16.2.2]docosa 1(21), 18(22), 19-triene-4-carboxylic acid amide

Fmoc-Cys(Trt)-OH, Fmoc-Val-OH, Fmoc-Ala-OH, Fmoc-His(trt)-OH and Fmoc-1-Phe-OH were sequentially coupled to 800 mg of Knorr resin using standard solid phase techniques. The resin was capped with acetic anhydride using the standard procedure. The resin was shaken 10 times with 10 mL of a freshly made solution of dichloromethane/trifluoroacetic acid/triethylsilane (96/2/2) for 10 minutes. The resin was thoroughly washed with dichloromethane (3 bed volumes) and MeOH (3 bed volumes). The resin was transferred to a scintillation vial. The vial was charged with dimethylacetamide (10 mL). Nitrogen was vigorously bubbled through the suspension for 10 minutes. Pd₂ dba₃ (91.5 mg, 0.25 equiv), dppf (222 mg, 1.0 equiv) and diisopropylethylamine (0.66 mL, 10.0 equiv) were sequentially added. The reaction was heated at 60° C. for 3 hours under nitrogen. The resin was filtered and thoroughly washed with DMF (5 bed volumes), dichloromethane (5 bed volumes), a 5% solution of diethyldithiocarbamate in DMF (5 bed volumes), dichloromethane (5 bed volumes) and MeOH (5 bed volumes). The beads were thoroughly dried under high vacuum. A freshly made solution of TFA/H₂O/Triisopropylsilane/anisole (92.5/2.5/2.5/2.5) (40 mL/g of resin) was added to the peptide synthesis vessel containing the beads. The reaction was gently stirred for 2 hrs and filtered. The beads were washed with 3 bed volumes of TFA. The cleavage sequence was repeated an additional time. The combined filtrates were evaporated to dryness under reduced pressure. The peptide was precipitated out using diisopropylether. The white solid (90 mg) was purified by HPLC. The desired material (10 mg) was isolated as the trifluoroacetate salt.

¹H NMR (400 MHz, DEUTERIUM OXIDE) δ ppm 0.80-0.96 (m, 6H) 1.10 (d, J=6.1 Hz, 3H) 1.91-1.99 (m, 4H) 2.76-3.87 (m, 6H) 4.02 (d, J=8.6 Hz, 1H) 4.12-4.71 (m, 4H) 7.12-7.42 (m, 5H) 8.52-8.66 (m, 1H)

LCMS: 615 (MH⁺)

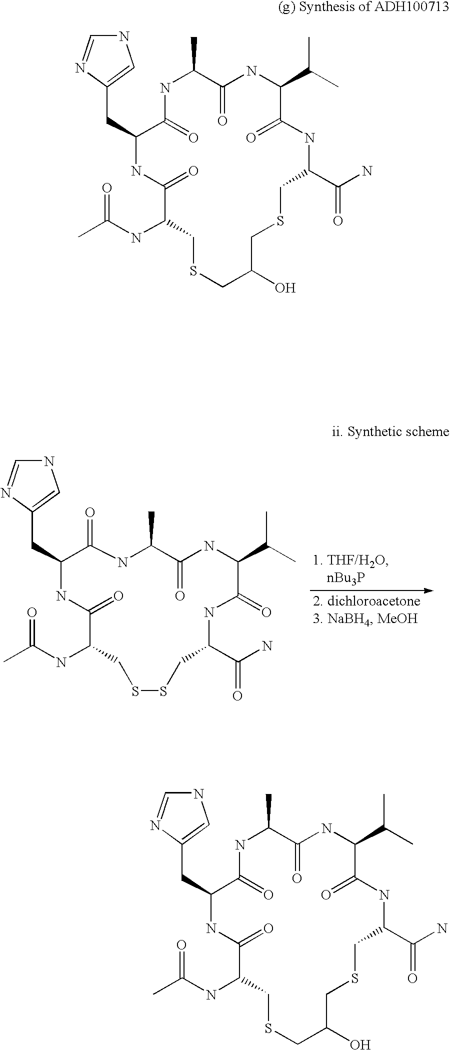

### 3R, 6S, 9S, 12S, 15R 15-Acetylamino-19-hydroxy-12-(1H-imidazol-4-ylmethyl)-6-isopropyl-9-methyl-5,8,11,14-tetraoxo-1,17-dithia-4,7,10,13-tetraaza-cycloicosane-3-carboxylic acid amide

A round bottom flask was charged with ADH1 (400 mg, 1.0 equiv), THF (40 mL) and water (40 mL). Tributylphosphine (0.26 mL, 1.5 equiv) was added in one portion to the mixture. The reaction was stirred for 90 min at room temperature. NaHCO₃ (176 mg, 3.0 equiv) was added to the reaction. After 15 min of stirring, dichloroacetone (93 mg, 1.05 equiv) was added and the reaction was stirred at room temperature for 90 minutes. MeOH (20 mL) and NaBH₄ (53 mg, 2.0 equiv) were sequentially added to the reaction. After 20 minutes, acetone (2 mL) was added. The reaction was concentrated under reduced pressure to 50 mL and purified directly by HPLC. The more polar compound (110 mg) was isolated as the trifluoroacetate salt.

¹H NMR (400 MHz, DEUTERIUM OXIDE) δ ppm 0.91 (d, J=6.7 Hz, 3H) 0.97 (d, J=6.7 Hz, 3H) 1.95 (s, 3H) 1.96-2.07 (m, 1H) 2.61-3.43 (m, 10H) 3.85-3.95 (m, 1H) 3.99 (d, J=8.7 Hz, 1H) 4.32-4.43 (m, 1H) 4.43-4.60 (m, 2H) 4.76-4.83 (m, 1H) 7.26 (s, 1H) 8.58 (s, 1H)

LCMS: 629 (MH⁺)

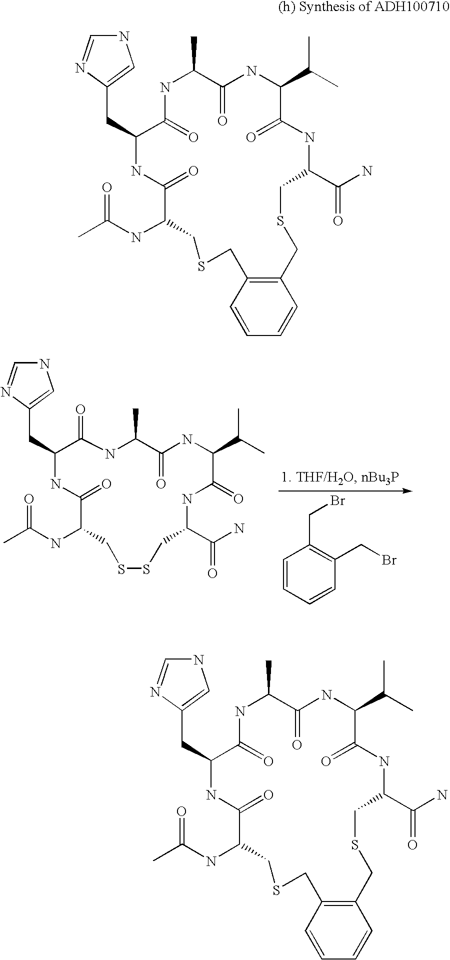

### 8R, 11S, 14S, 17S, 20R 20-Acetylamino-17-(1H-imidazol-4-ylmethyl)-11-isopropyl-14-methyl-10,13,16,19-tetraoxo-5,7,8,10,11,12,13,14,15,16,17,18,19,20,21,23-hexadecahydro-9H-6,22-dithia-9,12,15,18-tetraaza-benzocyclohenicosene-8-carboxylic acid amide

A round bottom flask was charged with ADH1 (520 mg, 1.0 equiv), THF (50 mL) and water (50 mL). Tributylphosphine (0.34 mL, 1.5 equiv) was added in one portion to the mixture. The reaction was stirred for 90 min at room temperature. NaHCO₃ (230 mg, 3.0 equiv) was added to the reaction. After 15 min of stirring, α,α′dibromo-ortho-xylene (264 mg, 1.1 equiv) in THF (10 mL) was added and the reaction was stirred at room temperature for 90 minutes. The reaction was concentrated under reduced pressure to 50 mL and purified by HPLC. The desired material (160 mg) was isolated as the trifluoroacetate salt.

¹H NMR (400 MHz, DEUTERIUM OXIDE) δ ppm 0.91 (d, J=6.7 Hz, 3H) 0.97 (d, J=6.7 Hz, 3H) 1.35 (d, J=7.1 Hz, 3H) 1.95 (s, 3H) 1.99-2.10 (m, 1H) 2.80 (d, J=7.4 Hz, 2H) 2.90 (dd, J=14.0, 9.4 Hz, 1H) 3.06-3.19 (m, 2H) 3.40 (dd, J=15.6, 4.7 Hz, 1H) 3.81-4.01 (m, 5H) 4.38 (t, J=7.4 Hz, 1H) 4.47 (q, J=7.1 Hz, 1H) 4.52 (dd, J=9.3, 3.9 Hz, 1H) 4.82 (dd, J=9.6, 4.7 Hz, 1H) 7.26 (s, 1H) 7.27-7.34 (m, 3H) 7.37-7.43 (m, 1H) 8.58 (s, 1H)

LCMS: 675 (MHz⁺⁾

### Example 2

### Improved Stability of Modified ADH-1 Cyclic Peptides

This Example evaluates the stability of various classes of chemically modified ADH-1 cyclic peptides synthesized as described in Example 1. As summarized below, certain classes of modifications were found to result in significantly improved stability of the modified ADH-1 compounds relative to unmodified ADH-1.

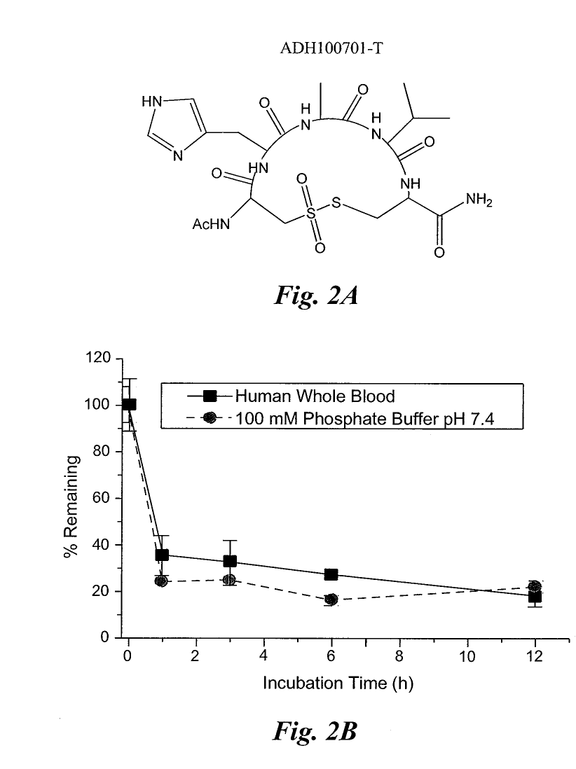

One class of ADH-1 modifications involved the oxidation of one or both sulfur atoms of the disulfide bond (e.g. —SO₂—S— or —S—SO₂—) to form thiosulfonates. FIG. 2A shows the structure of the modified compound, ADH100701-T, while FIG. 2B shows the results of stability testing. This modification did not result in an improvement in stability in PBS or human whole blood.

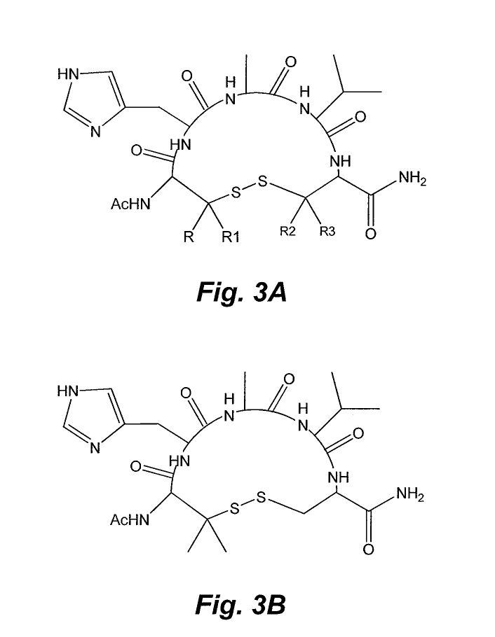

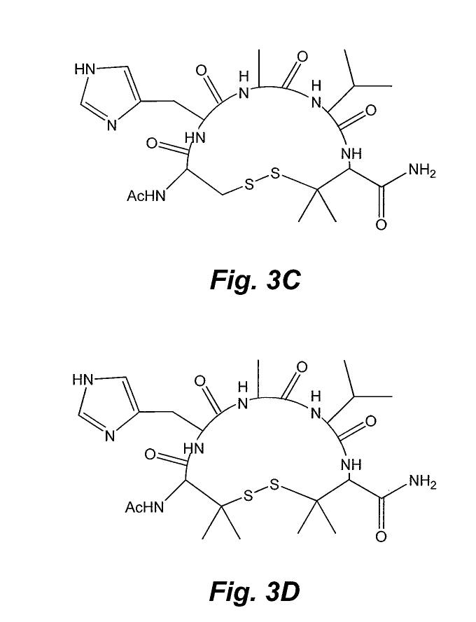

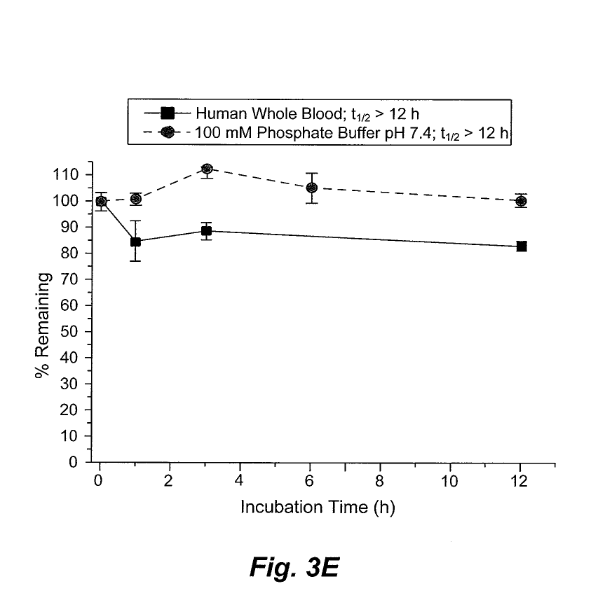

As shown in FIG. 3, incorporation of steric R groups adjacent or in the vicinity of the disulfide bond resulted in significantly improved stability in blood. Incorporating steric bulk adjacent the disulfide linkage in compound ADH100703-T (FIG. 3B) created an extraordinary stable molecule in whole blood. ADH100715-T (FIG. 3C) was similarly stable.

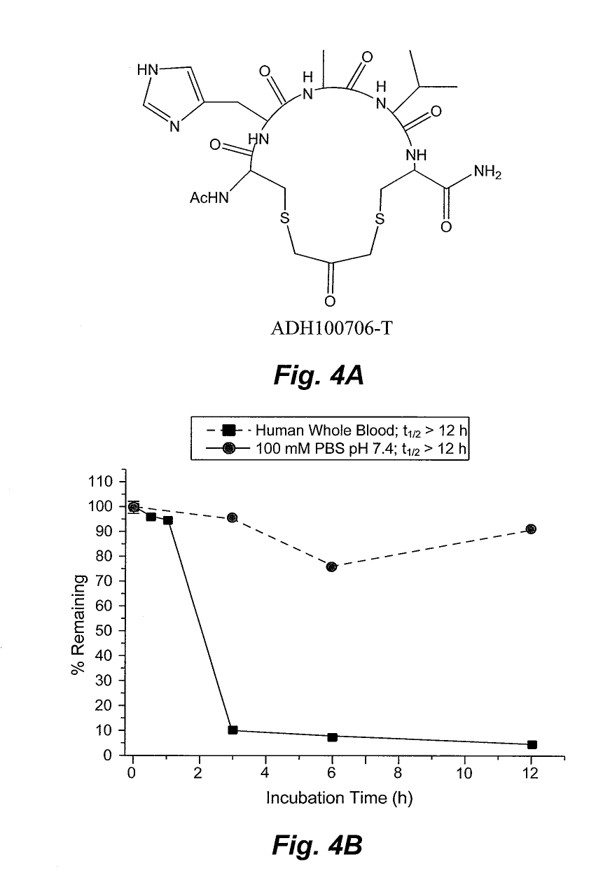

As shown in FIG. 4, dithioalkylation of the disulfide bond to form an acetone derivative failed to result in improved stability for compound ADH100706-T.

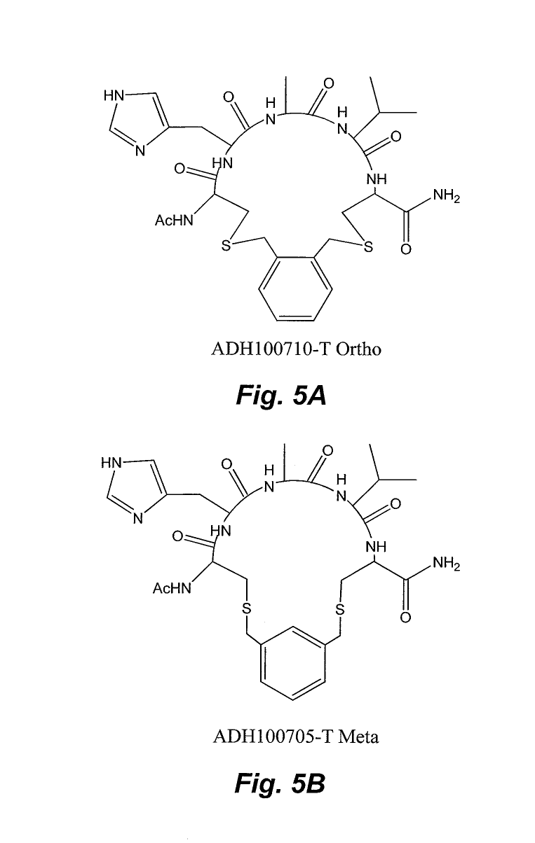

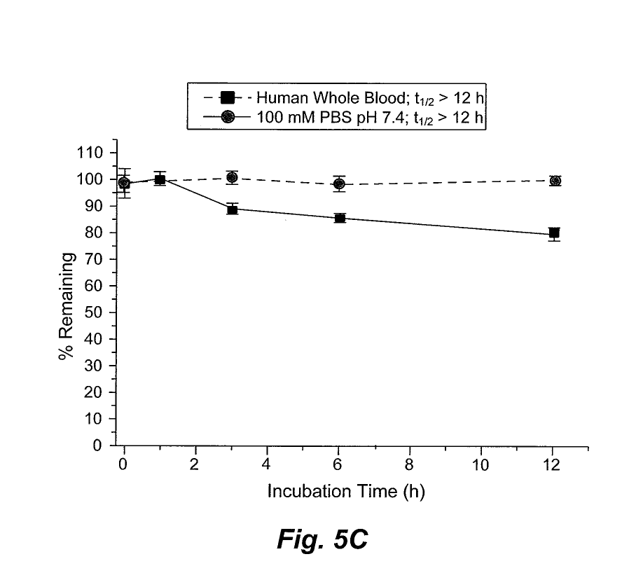

As shown in FIG. 5, dithioalkylation of the disulfide bond to produce aromatic derivatives resulted in improved stability for certain compounds. ADH100710-T was stable in PBS and blood, however ADH100705-T was much less so. ADH100705-T had relatively low aqueous solubility and ADH100710-T had aqueous solubility similar to ADH-1.

Dithiolalkylations of the disulfide bond to produce acetone derivatives failed to result in improved stability (not shown).

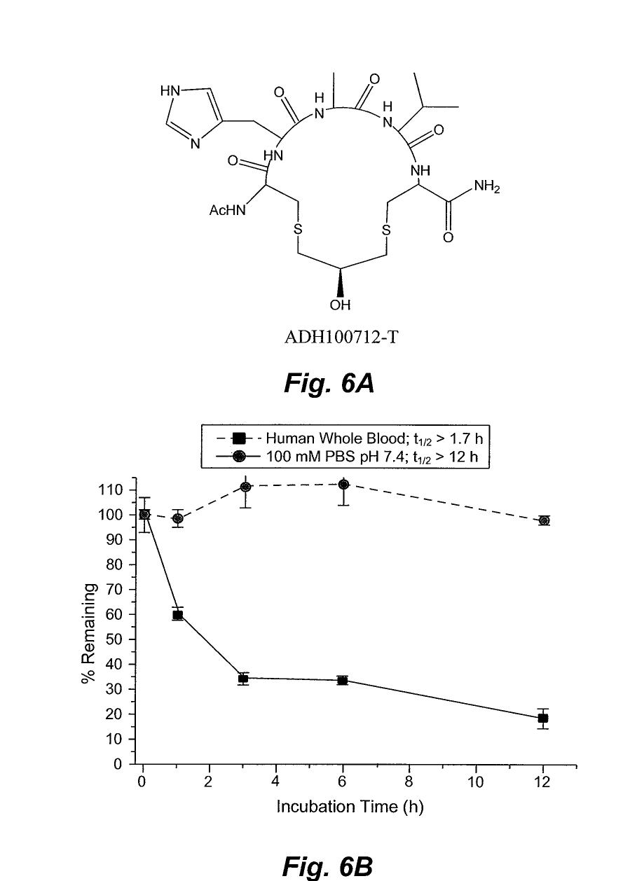

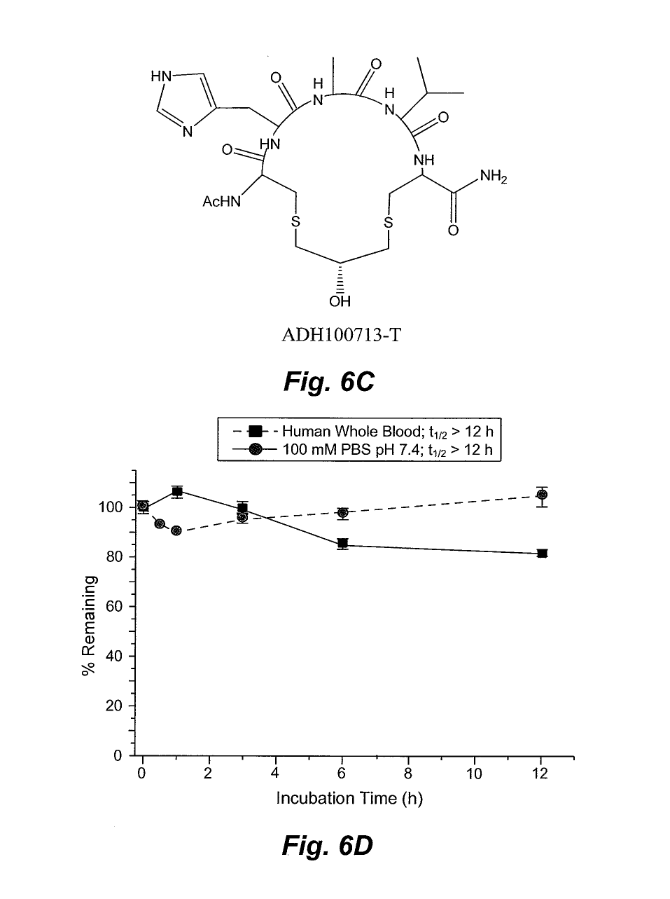

As shown in FIG. 6, certain dithioalkylation modifications of the disulfide bond to produce alcohol derivatives resulted in improved stability. Interestingly, while ADH-100712-T was stable in PBS, it failed to show improved stability in whole blood (FIG. 6A-B). In contrast, ADH100713-T was stable in both blood and PBS (FIGS. 6C-D).

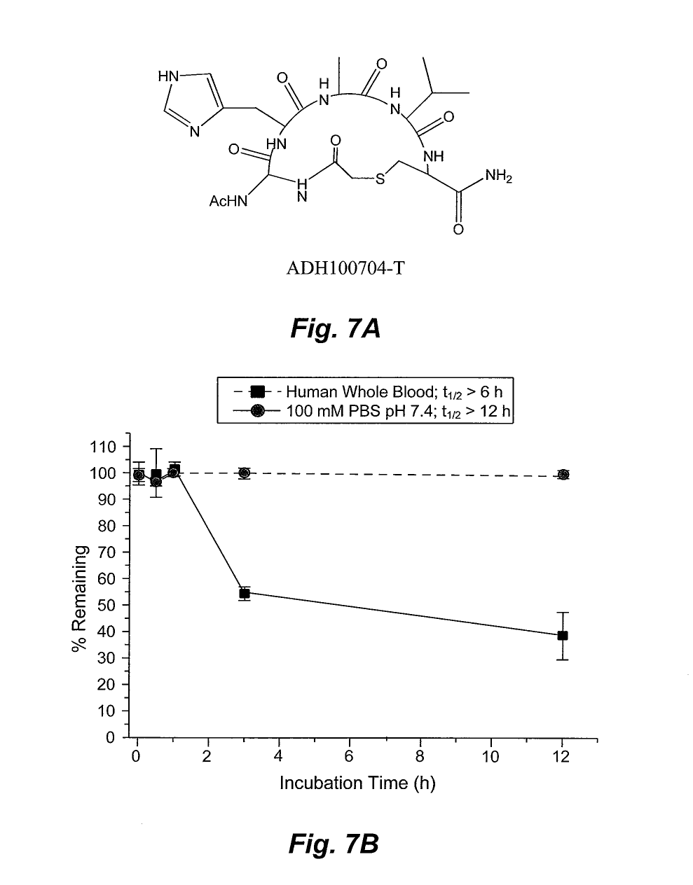

As shown in FIG. 7, modification involving the replacement of a sulfur atom of the disulfide bond to create simple thioether derivatives yielded a compound stable in PBS but did not significantly improve stability in blood. FIG. 7A shows the structure of the modified compound, ADH100704-T, while FIG. 7B shows the results of stability testing.

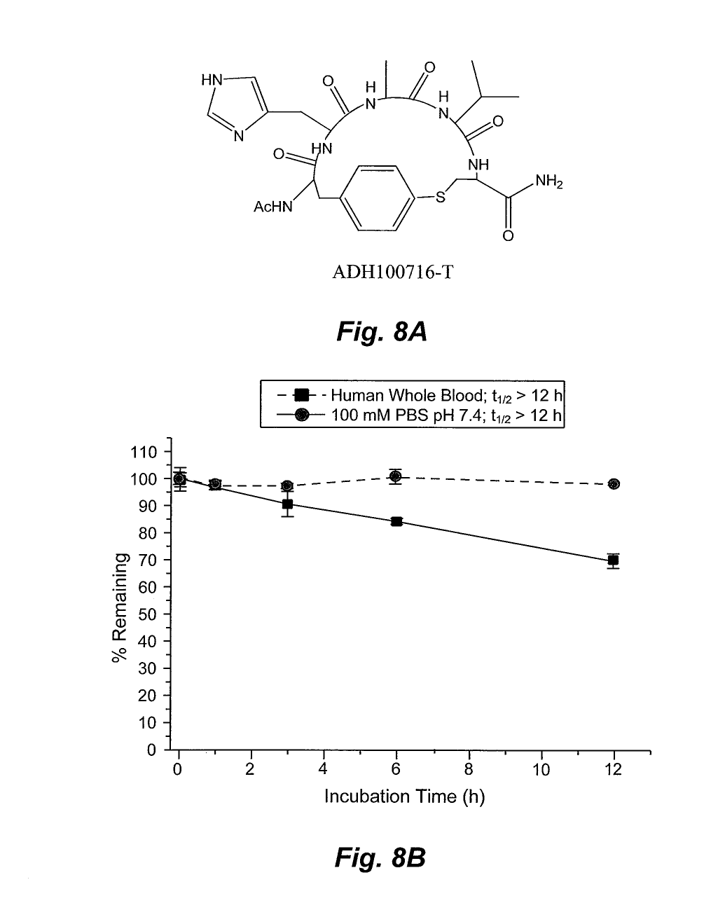

As shown in FIG. 8, modifications involving sulfur substitutions to create aromatic thioether derivatives, such as for compound ADH-100716-T (FIG. 8A), resulted in improved blood stability (FIG. 8B).

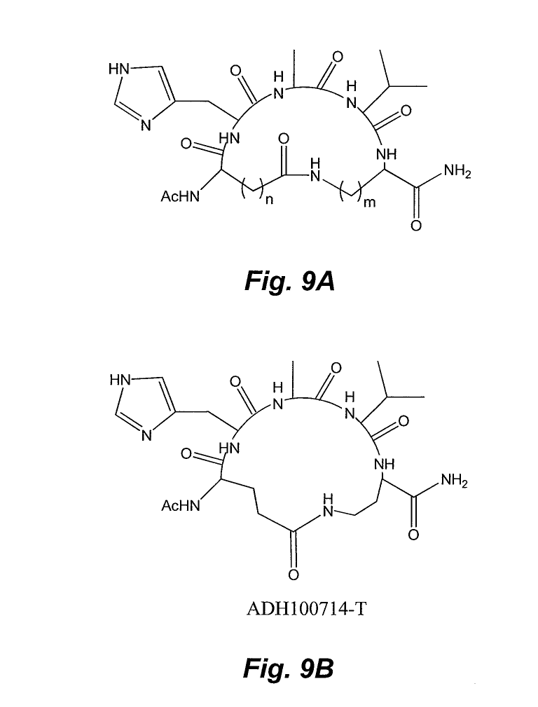

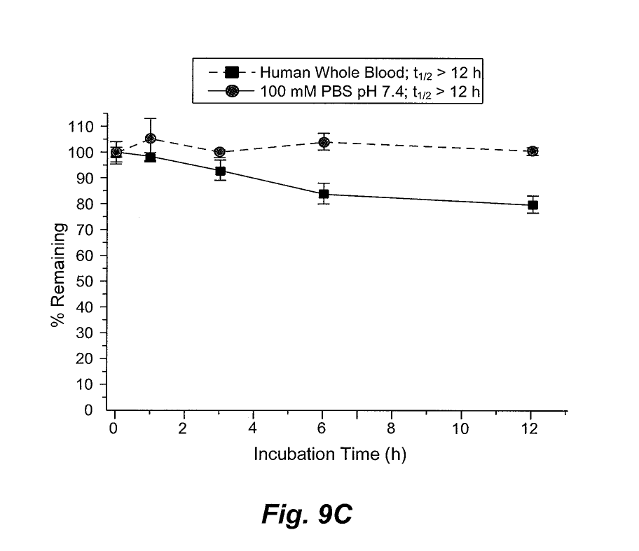

As shown in FIG. 9, certain amide derivatives in which both sulfur atoms of the disulfide bond were replaced resulted in improved stability in blood. Dab derivative (ADH100714-T; FIG. 9B) was stable in PBS and blood. A lysine derivative (ADH100711-T; not shown) was stable in PBS but only moderately stable in blood. An ornithine derivative (ADH100718-T; not shown) did not show improved stability in blood.

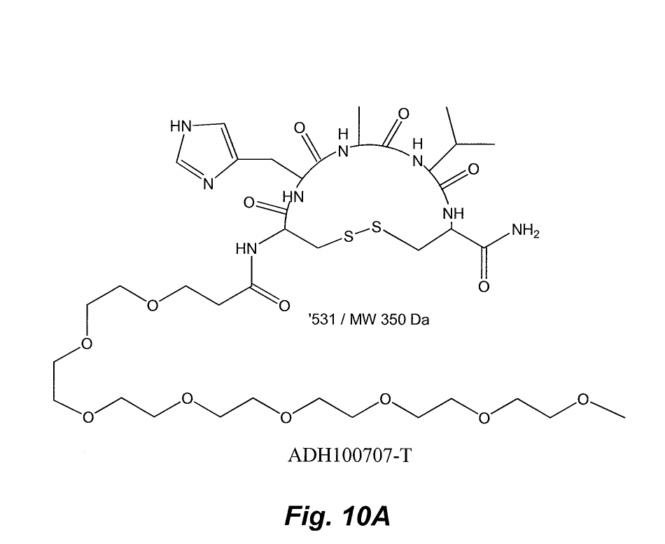

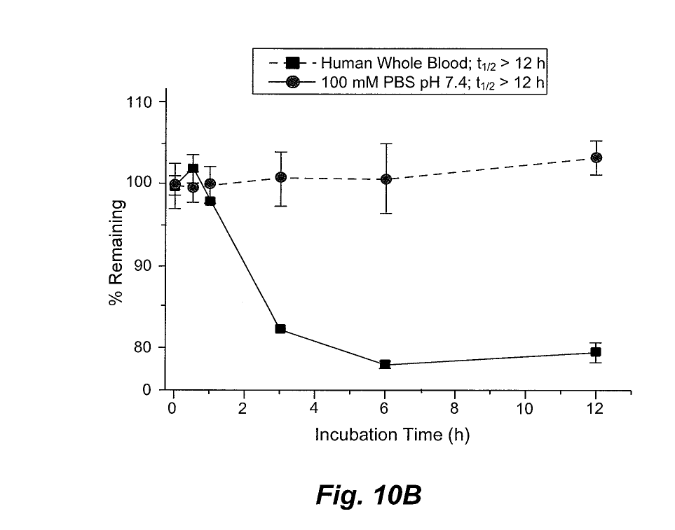

As shown in FIG. 10, ADH-1 which was pegylated, for example at the N-acetyl group of the molecule (ADH100707-T; PEG MW ˜350Da; FIG. 10A), resulted in improved stability in PBS and whole blood (FIG. 10B).

Therefore, significant improvements in stability of cyclic peptides were achieved using the chemical modifications described herein. While the blood half-life of unmodified ADH-1 is only about 2 hours, modified compounds of the present invention possessed half-lives of greater than 12 hours.

1. A cyclic peptide or salt thereof that comprises an intramolecular covalent disulfide bond between two non-adjacent residues and at least one cell adhesion recognition (CAR) sequence; wherein the disulfide bond has been modified as set forth in structures (A)-(E) below: 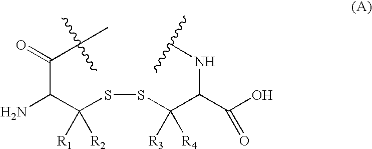
wherein R₁, R₂, R₃ and R₄ are the same or different and independently selected from H, alkyl, alkenyl, alkynyl, cycloalkyl, cycloalkyl-alkyl, heterocyclyl, heterocyclyl-alkyl, aryl, alkylaryl, arylalkyl, heteroaryl, alkylheteroaryl, heteroarylalkyl, alkoxy, alkylalkoxy, alkylthio, alkylamino, carbocycle, substituted carbocycle, heterocycle, substituted heterocycle, or —(CH₂)ₙ—X—(CH₂)ₘ— where n and m are independently 1, 2, 3 or 4 and X═O, S, NH, or N-alkyl; 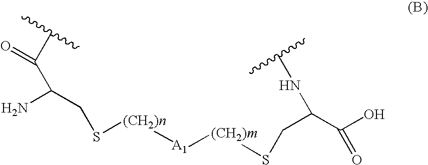
wherein n and m are the same or different and independently 1, 2, 3 or 4 and A₁ is aryl, substituted aryl, heteraryl, substituted heteroaryl, carbonyl, a carbon bearing an alcohol, a carbon bearing an alkylether, a carbon bearing an arylether, a carbon bearing an heteroarylether, a carbon bearing an alkylester, a carbon bearing an arylester, a carbon bearing an heteroarylester, a carbon bearing an amine, a carbon bearing an alkylamine, a carbon bearing an arylamine, a carbon bearing a heteroarylamine, a carbon bearing an amide, or a carbon bearing an alkylamide; 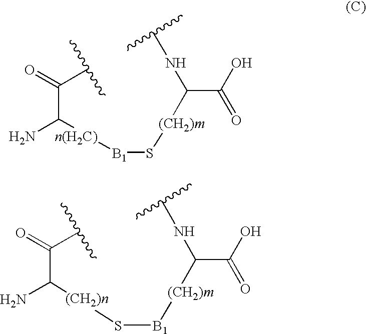
wherein n and m are independently 1, 2, 3 or 4; and B₂ is aryl or heteroaryl; 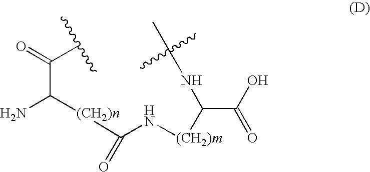
wherein n and m are independently 1, 2, 3 or 4; or 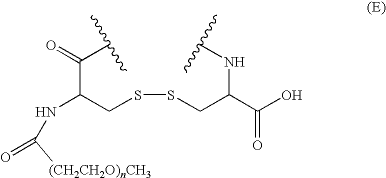
wherein n is in a range from about 1 to 100, and wherein the cyclic peptide is optionally acetylated at the N-terminus and/or amidated at the C-terminus.

2. A cyclic peptide according to claim 1, wherein the modified disulfide bond has a structure selected from:

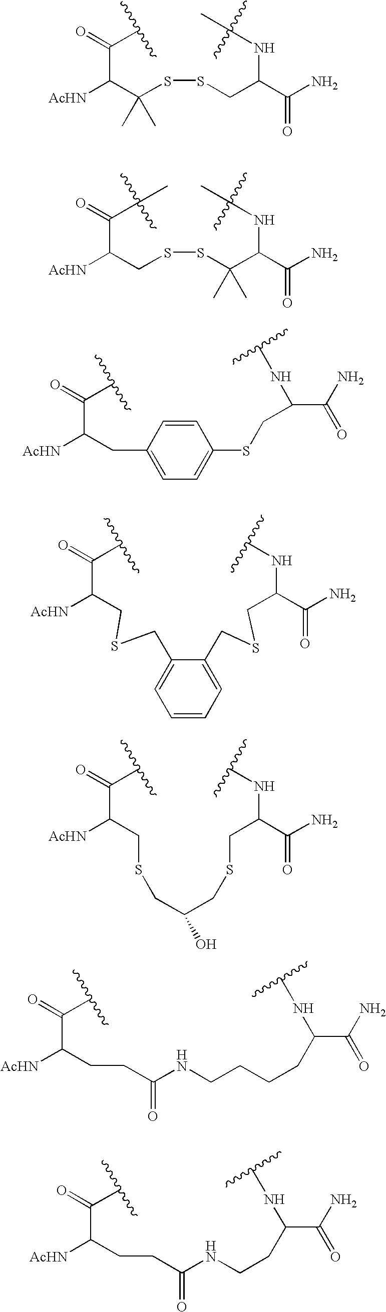

3. A cyclic peptide according to claim 1, wherein the CAR comprises the sequence HAV.

4. A cyclic peptide according to claim 1, wherein the CAR comprises the sequence HAV and the cyclic peptide has the following formula: 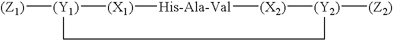
wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; and wherein a covalent disulfide bond is formed between residues Y₁ and Y₂ which is modified as set forth in any one of (A)-(E) of claim 1, and wherein Z₁ and Z₂ are optional, and if present, are independently selected from the group consisting of amino acid residues and combinations thereof in which the residues are linked by peptide bonds.

5. A cyclic peptide according to claim 1, wherein the CAR comprises the sequence: Asp/Glu-Trp-Val-Ile/Val/Met-Pro/Ala-Pro (SEQ ID NO:40), wherein “Asp/Glu” is an amino acid that is either Asp or Glu, “Ile/Val/Met” is an amino acid that is Ile, Val or Met, and “Pro/Ala” is either Pro or Ala.

6. A cyclic peptide according to claim 1, wherein the CAR comprises the sequence: Gly/Asp/Ser-Trp-Val/Ile/Met-Trp-Asn-Gln (SEQ ID NO: 268), wherein “Gly/Asp/Ser” is an amino acid that is Gly, Asp or Ser; and “Val/Ile/Met” is an amino acid that is Val, Ile or Met.

7. A cyclic peptide according to claim 1, wherein the CAR comprises the sequence: (a) Ile/Val-Phe-Aaa-Ile-Baa-Caa-Daa-Ser/Thr-Gly-Eaa-Leu/Met (SEQ ID NO: 182), wherein Aaa, Baa, Caa, Daa and Eaa are independently selected from the group consisting of amino acid residues; or comprises the sequence Trp-Leu-Aaa-Ile-Asp/Asn-Baa-Caa-Daa-Gly-Gln-Ile (SEQ ID NO:183), wherein Aaa, Baa, Caa and Daa are independently selected from the group consisting of amino acid residues.

8. A cyclic peptide according to claim 1, wherein the CAR comprises the sequence: Aaa-Phe-Baa-Ile/Leu/Val-Asp/Asn/Glu-Caa-Daa-Ser/Thr/Asn-Gly (SEQ ID NO:211) wherein Aaa, Baa, Caa and Daa are independently selected from amino acid residues; Ile/Leu/Val is an amino acid that is selected from the group consisting of isoleucine, leucine and valine, Asp/Asn/Glu is an amino acid that is selected from the group consisting of aspartate, asparagine and glutamate; and Ser/Thr/Asn is an amino acid that is selected from the group consisting of serine, threonine or asparagine.

9. A pharmaceutical compositions comprising a cyclic peptide according to claim 1 in combination with a physiologically acceptable carrier.

10. A pharmaceutical compositions comprising a cyclic peptide according to claim 1, a physiologically acceptable carrier and one or more anticancer agents.

11. A pharmaceutical composition according to claim 10, wherein the one or more anticancer agents are selected from the group consisting of alkylating agents, antimetabolites, natural products, antibiotics, plantinum compounds, anthracenediones, methylhydrazine derivatives, adrenocortical suppressants, tyrosine kinase inhibitors, multi-targeted kinase inhibitors, adrenocorticosteroids, estrogens, progestins, aromatase inhibitors, antiestrogens, antitumor antibodies and radiation therapy.

12. A method for treating cancer comprising administering to a subject in need thereof a pharmaceutical composition according to any one of claims 9-11.

13. A method for treating a condition or disease mediated by cell adhesion comprising administering to a subject in need thereof a pharmaceutical composition according to any one of claims 9-11.
